(19) United States
(12) Patent Application Publication
(10) Pub. No.: US 2024/0139169 A1
(43) Pub. Date: May 2, 2024

(54) METHODS AND COMPOSITIONS FOR TREATING AGITATION

(71) Applicant: BioXcel Therapeutics, Inc., New Haven, CT (US)

(72) Inventors: Michael DE VIVO, New Haven, CT (US); Friso POSTMA, New Haven, CT (US); David Christian HANLEY, Brookfield, CT (US); Vasukumar KAKUMANU, Guntur (IN); Subhendu SETH, Gurgaon (IN); Dinesh Kumar DHULL, Gurgaon (IN)

(21) Appl. No.: 18/278,786

(22) PCT Filed: Feb. 25, 2022
(86) PCT No.: PCT/US2022/017963

§ 371 Date: Aug. 24, 2023

Related U.S. Application Data
(60) Provisional application No. 63/154,281, filed on Feb. 26, 2021.

(51) Int. Cl. A61K 31/444; A61K 9/00; A61K 31/4174; A61P 25/18

(52) U.S. Cl. CPC A61K 31/444; A61K 9/0056; A61K 31/4174; A61P 25/18

(57) ABSTRACT

The present disclosure relates to the treatment of agitation caused by noradrenergic hyperarousal in an agitated subject. The present disclosure provides oromucosal dosage forms comprising effective amounts of latrepirdine either alone or in combination with dexmedetomidine or pharmaceutically acceptable salts thereof and one or more pharmaceutically acceptable carriers and/excipients. Methods of their use are also provided. The present disclosure also provides a method of treating depression by administering oromucosally a therapeutically effective amount of dexmedetomidine alone or in combination with latrepirdine.

### CROSS-REFERENCE TO RELATED APPLICATIONS

This application claims priority to and the benefit of U.S. Provisional Application No. 63/154,281, filed Feb. 26, 2021, the contents of which are hereby incorporated in their entirety as if set forth herein.

### FIELD

The present disclosure relates to the treatment of agitation in a subject in need thereof by administering an oromucosal dosage form comprising an effective amount of latrepirdine either alone or in combination with an effective amount of dexmedetomidine or pharmaceutically acceptable salts thereof and one or more pharmaceutically acceptable carriers and/excipients. The present disclosure also provides a method of treating depression in a subject in need thereof, the method comprising administering oromucosally a therapeutically effective amount of dexmedetomidine alone or in combination with latrepirdine. The disclosure further relates to treatment of other disorders associated with noradrenergic hyperarousal.

### BACKGROUND

Agitation is a complex biological phenomenon that represents a loss of behavioral control. A single drug is not effective against all types of agitation. Many neural pathways and associated receptors mediate aspects of agitation. These pathways include amygdala, frontal cortex, nucleus acumbens and locus coeruleus. Drugs and associated drug targets to treat agitation work in these brain areas include antipsychotics and dopamine D2 receptors, benzodiazepines and GABA receptors, ketamine and glutamate receptors, and serotonin uptake inhibitors (Miller C W, Hodzic V, Weintraub E. Current Understanding of the Neurobiology of Agitation. Western Journal of Emergency Medicine. 2020 July; 21(4): 841). Depression is a mood disease that causes a persistent feeling of sadness and loss of interest that results from a complex interaction of social, psychological, and biological factors. During a depressive episode, the person experiences a depressed mood (e.g. feeling sad, irritable, or empty) or a loss a pleasure or interest in activities, for most of the day, nearly every day, for at least two weeks. A depressive episode can be categorized as mild, moderate, or severe depending on the number and severity of symptoms, as well as the impact on the individual's functioning. Increasingly there is a large scientific and medical effort to develop drugs that target specific pathways and therefore treat specific aspects of agitation.

### SUMMARY

The present disclosure provides oromucosal dosage forms comprising effective amounts of latrepirdine either alone or in combination with dexmedetomidine or pharmaceutically acceptable salts thereof and one or more pharmaceutically acceptable carriers and/or excipients. The present disclosure provides methods of treating disorders associated with noradrenergic mediated hyperarousal in a subject, comprising oromucosally administering to the subject a therapeutically effective amount of latrepirdine or a pharmaceutically acceptable salt thereof

The present disclosure provides methods of treating agitation in a subject, comprising administering to the subject a therapeutically effective amount of latrepirdine or a pharmaceutically acceptable salt thereof.

The present disclosure also provides methods of treating agitation in an agitated subject, comprising administering to the subject a therapeutically effective amount of latrepirdine or a pharmaceutically acceptable salt thereof. The present disclosure also provides methods of treating acute agitation in a subject, comprising administering to the subject a therapeutically effective amount of latrepirdine or a pharmaceutically acceptable salt thereof. The present disclosure also provides methods of treating acute agitation in an agitated subject, comprising administering to the subject a therapeutically effective amount of latrepirdine or a pharmaceutically acceptable salt thereof. The present disclosure also provides methods of treating or preventing chronic agitation in a subject, comprising administering to the subject a therapeutically effective amount of latrepirdine or a pharmaceutically acceptable salt thereof. The present disclosure also provides methods of treating or preventing chronic agitation in an agitated subject, comprising administering to the subject a therapeutically effective amount of latrepirdine or a pharmaceutically acceptable salt thereof.

The present disclosure also provides a method of treating agitation in an agitated subject, the method comprising administering to the subject a therapeutically effective amount of dexmedetomidine or a pharmaceutically acceptable salt thereof in combination with a therapeutically effective amount of latrepirdine or a pharmaceutically acceptable salt thereof. In embodiments, the agitation is caused by noradrenergic hyperarousal. In embodiments, the agitated subject also exhibits aggression. In embodiments, the agitation is treated without also inducing significant sedation. The present disclosure also provides a method of treating acute agitation in an agitated subject, the method comprising administering to the subject a therapeutically effective amount of dexmedetomidine or a pharmaceutically acceptable salt thereof in combination with a therapeutically effective amount of latrepirdine or a pharmaceutically acceptable salt thereof. The present disclosure also provides a method of treating chronic agitation in a subject, the method comprising administering to the subject a therapeutically effective amount of dexmedetomidine or a pharmaceutically acceptable salt thereof in combination with a therapeutically effective amount of latrepirdine or a pharmaceutically acceptable salt thereof.

In embodiments, the present disclosure provides a method of treating agitation in an agitated subject, comprising administering oromucosally (e.g. sublingually, buccally, or gingivally), to the subject a dosage form comprising a therapeutically effective amount of latrepirdine or a pharmaceutically acceptable salt thereof. In embodiments, the present disclosure provides a method of treating agitation in an agitated subject, comprising administering oromucosally (e.g., sublingually, buccally, or gingivally) to the subject a dosage form comprising about 1 mg to about 100 mg of latrepirdine or a pharmaceutically acceptable salt thereof. In embodiments, the present disclosure provides a method of treating agitation in an agitated subject, comprising administering oromucosally (e.g., sublingually, buccally, or gingivally) to the subject a dosage form comprising about 10 mg to about 60 mg of latrepirdine or a pharmaceutically acceptable salt thereof, e.g. about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 35 mg, about 40 mg, about 45 mg, about 50 mg, about 55 mg, or about 60 mg, including all ranges and values in between.

In embodiments, about 10 mg of latrepirdine or a pharmaceutically acceptable salt thereof is administered once a day. In embodiments, about 10 mg of latrepirdine or a pharmaceutically acceptable salt thereof is administered twice a day. In embodiments, about 10 mg of latrepirdine or a pharmaceutically acceptable salt thereof is administered thrice a day. In embodiments, about 20 mg of latrepirdine or a pharmaceutically acceptable salt thereof is administered once a day. In embodiments, about 20 mg of latrepirdine or a pharmaceutically acceptable salt thereof is administered twice a day. In embodiments, about 20 mg of latrepirdine or a pharmaceutically acceptable salt thereof is administered thrice a day. In embodiments, about 30 mg of latrepirdine or a pharmaceutically acceptable salt thereof is administered once a day. In embodiments, about 30 mg of latrepirdine or a pharmaceutically acceptable salt thereof is administered twice a day. In embodiments, about 30 mg of latrepirdine or a pharmaceutically acceptable salt thereof is administered thrice a day. In embodiments, a total daily dose of about 10 mg of latrepirdine or a pharmaceutically acceptable salt thereof is administered to the subject. In embodiments, a total daily dose of about 20 mg of latrepirdine or a pharmaceutically acceptable salt thereof is administered to the subject. In embodiments, a total daily dose of about 30 mg of latrepirdine or a pharmaceutically acceptable salt thereof is administered to the subject. In embodiments, a total daily dose of about 40 mg of latrepirdine or a pharmaceutically acceptable salt thereof is administered to the subject. In embodiments, a total daily dose of about 60 mg of latrepirdine or a pharmaceutically acceptable salt thereof is administered to the subject.

In embodiments, the present disclosure provides methods of treating noradrenergic mediated hyperarousal in a subject, comprising administering oromucosally to the subject a dosage form comprising:
(i) a therapeutically effective amount of latrepirdine or a pharmaceutically acceptable salt thereof and
(ii) a therapeutically effective amount of dexmedetomidine or a pharmaceutically acceptable salt thereof (e.g. hydrochloride).

In embodiments, the oromucosal administration includes sublingual, buccal, or gingival administration.

In embodiments, the present disclosure provides a method of treating agitation in an agitated subject, comprising administering oromucosally to the subject a dosage form comprising:
(i) a therapeutically effective amount of latrepirdine or a pharmaceutically acceptable salt thereof, and
(ii) a therapeutically effective amount of dexmedetomidine or a pharmaceutically acceptable salt thereof (e.g., dexmedetomidine hydrochloride).

In embodiments, the present disclosure provides a method of treating agitation in an agitated subject, comprising administering about 1 mg to about 500 mg of latrepirdine or a pharmaceutically acceptable salt thereof and about 5 micrograms to about 360 micrograms of dexmedetomidine or a pharmaceutically acceptable salt thereof to the subject. In embodiments, the present disclosure provides a method of treating agitation in an agitated subject, comprising administering oromucosally about 1 mg to about 100 mg of latrepirdine or a pharmaceutically acceptable salt thereof and about 10 micrograms to about 300 micrograms of dexmedetomidine or a pharmaceutically acceptable salt thereof to the subject. In embodiments, the present disclosure provides a method of treating agitation in an agitated subject, comprising oromucosally administering about 5 mg to about 100 mg of latrepirdine or a pharmaceutically acceptable salt thereof and about 20 micrograms to about 300 micrograms of dexmedetomidine or a pharmaceutically acceptable salt thereof to the subject. In embodiments, the present disclosure provides a method of treating agitation in an agitated subject, comprising administering oromucosally about 5 mg to about 60 mg of latrepirdine or a pharmaceutically acceptable salt thereof and about 10 micrograms to about 200 micrograms of dexmedetomidine or a pharmaceutically acceptable salt thereof to the subject.

In embodiments, the present disclosure provides a method of treating agitation in an agitated subject, comprising administering about 10 mg of latrepirdine or a pharmaceutically acceptable salt thereof and about 30 micrograms of dexmedetomidine or a pharmaceutically acceptable salt thereof to the subject.

In embodiments, the present disclosure provides a method of treating agitation in an agitated subject, comprising administering about 10 mg of latrepirdine or a pharmaceutically acceptable salt thereof and about 30 micrograms of dexmedetomidine or a pharmaceutically acceptable salt thereof to the subject, wherein the agitation is caused by noradrenergic hyperarousal.

In embodiments, the present disclosure provides a method of treating agitation in an agitated subject, comprising administering about 20 mg of latrepirdine or a pharmaceutically acceptable salt thereof and about 30 micrograms of dexmedetomidine or a pharmaceutically acceptable salt thereof to the subject.

In embodiments, the present disclosure provides a method of treating agitation in an agitated subject, comprising administering about 20 mg of latrepirdine or a pharmaceutically acceptable salt thereof and about 30 micrograms of dexmedetomidine or a pharmaceutically acceptable salt thereof to the subject, wherein the agitation is caused by noradrenergic hyperarousal.

In embodiments, the present disclosure provides a method of treating agitation in an agitated subject, comprising administering about 10 mg of latrepirdine or a pharmaceutically acceptable salt thereof and about 40 micrograms of dexmedetomidine or a pharmaceutically acceptable salt thereof to the subject,

In embodiments, the present disclosure provides a method of treating agitation in an agitated subject, comprising administering about 10 mg of latrepirdine or a pharmaceutically acceptable salt thereof and about 40 micrograms of dexmedetomidine or a pharmaceutically acceptable salt thereof to the subject, wherein the agitation is caused by noradrenergic hyperarousal.

In embodiments, the present disclosure provides a method of treating agitation in an agitated subject, comprising administering about 20 mg of latrepirdine or a pharmaceutically acceptable salt thereof and about 40 micrograms of dexmedetomidine or a pharmaceutically acceptable salt thereof to the subject.

In embodiments, the present disclosure provides a method of treating agitation in an agitated subject, comprising administering about 20 mg of latrepirdine or a pharmaceutically acceptable salt thereof and about 40 micrograms of dexmedetomidine or a pharmaceutically acceptable salt thereof to the subject, wherein the agitation is caused by noradrenergic hyperarousal.

In embodiments, the present disclosure provides a method of treating agitation in an agitated subject, comprising administering about 10 mg of latrepirdine or a pharmaceutically acceptable salt thereof and about 60 micrograms of dexmedetomidine or a pharmaceutically acceptable salt thereof to the subject.

In embodiments, the present disclosure provides a method of treating agitation in an agitated subject, comprising administering about 10 mg of latrepirdine or a pharmaceutically acceptable salt thereof and about 60 micrograms of dexmedetomidine or a pharmaceutically acceptable salt thereof to the subject, wherein the agitation is caused by noradrenergic hyperarousal.

In embodiments, the present disclosure provides a method of treating agitation in an agitated subject, comprising administering about 20 mg of latrepirdine or a pharmaceutically acceptable salt thereof and about 60 micrograms of dexmedetomidine or a pharmaceutically acceptable salt thereof to the subject.

In embodiments, the present disclosure provides a method of treating agitation in an agitated subject, comprising administering about 20 mg of latrepirdine or a pharmaceutically acceptable salt thereof and about 60 micrograms of dexmedetomidine or a pharmaceutically acceptable salt thereof to the subject, wherein the agitation is caused by noradrenergic hyperarousal.

In embodiments, the present disclosure provides a method of treating agitation in an agitated subject, comprising administering about 10 mg of latrepirdine or a pharmaceutically acceptable salt thereof and about 80 micrograms of dexmedetomidine or a pharmaceutically acceptable salt thereof to the subject.

In embodiments, the present disclosure provides a method of treating agitation in an agitated subject, comprising administering about 10 mg of latrepirdine or a pharmaceutically acceptable salt thereof and about 80 micrograms of dexmedetomidine or a pharmaceutically acceptable salt thereof to the subject, wherein the agitation is caused by noradrenergic hyperarousal.

In embodiments, the present disclosure provides a method of treating agitation in an agitated subject, comprising administering about 20 mg of latrepirdine or a pharmaceutically acceptable salt thereof and about 80 micrograms of dexmedetomidine or a pharmaceutically acceptable salt thereof to the subject.

In embodiments, the present disclosure provides a method of treating agitation in an agitated subject, comprising administering about 20 mg of latrepirdine or a pharmaceutically acceptable salt thereof and about 80 micrograms of dexmedetomidine or a pharmaceutically acceptable salt thereof to the subject, wherein the agitation is caused by noradrenergic hyperarousal.

In embodiments, the present disclosure provides a method of treating agitation in an agitated subject, comprising administering about 10 mg of latrepirdine or a pharmaceutically acceptable salt thereof and about 120 micrograms of dexmedetomidine or a pharmaceutically acceptable salt thereof to the subject.

In embodiments, the present disclosure provides a method of treating agitation in an agitated subject, comprising administering about 10 mg of latrepirdine or a pharmaceutically acceptable salt thereof and about 120 micrograms of dexmedetomidine or a pharmaceutically acceptable salt thereof to the subject, wherein the agitation is caused by noradrenergic hyperarousal.

In embodiments, the present disclosure provides a method of treating agitation in an agitated subject, comprising administering about 20 mg of latrepirdine or a pharmaceutically acceptable salt thereof and about 120 micrograms of dexmedetomidine or a pharmaceutically acceptable salt thereof to the subject.

In embodiments, the present disclosure provides a method of treating agitation in an agitated subject, comprising administering about 20 mg of latrepirdine or a pharmaceutically acceptable salt thereof and about 120 micrograms of dexmedetomidine or a pharmaceutically acceptable salt thereof to the subject, wherein the agitation is caused by noradrenergic hyperarousal.

In embodiments, the present disclosure provides a method of treating agitation in an agitated subject, comprising administering about 10 mg of latrepirdine or a pharmaceutically acceptable salt thereof and about 180 micrograms of dexmedetomidine or a pharmaceutically acceptable salt thereof to the subject.

In embodiments, the present disclosure provides a method of treating agitation in an agitated subject, comprising administering about 10 mg of latrepirdine or a pharmaceutically acceptable salt thereof and about 180 micrograms of dexmedetomidine or a pharmaceutically acceptable salt thereof to the subject, wherein the agitation is caused by noradrenergic hyperarousal.

In embodiments, the present disclosure provides a method of treating agitation in an agitated subject, comprising administering about 20 mg of latrepirdine or a pharmaceutically acceptable salt thereof and about 180 micrograms of dexmedetomidine or a pharmaceutically acceptable salt thereof to the subject.

In embodiments, the present disclosure provides a method of treating agitation in an agitated subject, comprising administering about 20 mg of latrepirdine or a pharmaceutically acceptable salt thereof and about 180 micrograms of dexmedetomidine or a pharmaceutically acceptable salt thereof to the subject, wherein the agitation is caused by noradrenergic hyperarousal.

In embodiments, the present disclosure provides a method of treatment comprising administering dexmedetomidine or a pharmaceutically acceptable salt to a subject in an oromucosal dosage form that provides rapid relief of agitation and then continuing treatment with latrepirdine or a pharmaceutically acceptable salt for an effective period of time. In embodiments, latrepirdine and dexmedetomidine or pharmaceutically acceptable salts thereof are administered sublingually. In embodiments, latrepirdine and dexmedetomidine or pharmaceutically acceptable salts thereof are administered buccally. In embodiments, latrepirdine and dexmedetomidine or pharmaceutically acceptable salts thereof are administered sublingually or buccally as a tablet. In embodiments, the tablet is lyophilized. In embodiments, latrepirdine and dexmedetomidine or pharmaceutically acceptable salts thereof are administered sublingually or buccally or gingivally as a wafer. In embodiments, latrepirdine and dexmedetomidine or pharmaceutically acceptable salts thereof are administered sublingually or buccally or gingivally as a patch. In embodiments, latrepirdine and dexmedetomidine or pharmaceutically acceptable salts thereof are administered sublingually or buccally or gingivally as a film.

In embodiments, the present disclosure provides a synergistic combination comprising:
(i) a therapeutically effective amount of latrepirdine or a pharmaceutically acceptable salt thereof and
(ii) a therapeutically effective amount of dexmedetomidine or a pharmaceutically acceptable salt thereof, for the treatment of agitation in a subject in need thereof.

In embodiments, the agitation is associated with a neurodegenerative disorder selected from the group consisting of Alzheimer's disease, frontotemporal dementia (FTD), dementia, dementia with Lewy bodies (DLB), post-traumatic stress disorder (PTSD), Parkinson's disease, vascular dementia, vascular cognitive impairment, Huntington's disease, multiple sclerosis, Creutzfeldt-Jakob disease, multiple system atrophy, progressive supranuclear palsy and other related neurodegenerative disorders. In embodiments, the agitation is associated with sundown syndrome in Alzheimer's disease/dementia. In embodiments, the agitation is chronic and is associated with dementia.

In embodiments, the agitation is associated with a neuropsychiatric disease selected from the group consisting of schizophrenia, bipolar disorder, bipolar mania, delirium, and depression. In embodiments, the agitation is associated with an alcohol and substance abuse withdrawal including opioid withdrawal. In embodiments, the agitation is associated with an OPD/IPD procedure (e.g. MRI, CT or CAT scan, lumbar puncture, bone marrow aspiration/biopsy, tooth extraction or other dental procedures).

In embodiments, the agitation is caused by noradrenergic hyperarousal.

In embodiments, the agitation is treated without also inducing significant sedation.

In embodiments, the agitation is a result of administration of alpha-2 adrenergic receptors antagonist such as yohimbine. In embodiments, the agitation is caused as a result of administration of cocaine. In embodiments, the agitation is caused as a result of administration of lurasidone. In embodiments, the agitation is caused as a result of administration of mirtazapine. In embodiments, the agitation is caused as a result of administration of esmirtazapine. In embodiments, the agitation is caused as a result of administration of atipamezole. In embodiments, the agitation is caused as a result of administration of trazodone. In embodiments, the present disclosure provides a method of treating agitation in a subject in need thereof, comprising oromucosally administering to the subject a dosage form comprising a therapeutically effective amount of latrepirdine or a pharmaceutically acceptable salt thereof wherein said agitation is caused due to the administration of yohimbine.

In embodiments, the agitation may be acute or chronic. In embodiments, the agitation may be severe or mild. In embodiments, the agitation may be acute or chronic. In embodiments, the agitation may be severe or mild.

In embodiments, the present disclosure provides a method of treating depression in a subject in need thereof, comprising administering oromucosally to the subject a dosage form comprising a therapeutically effective amount of latrepirdine or a pharmaceutically acceptable salt thereof.

The present disclosure provides a method of treating depression in a subject in need thereof, the method comprising administering oromucosally to the subject a therapeutically effective amount of dexmedetomidine or a pharmaceutically acceptable salt thereof.

The present disclosure provides a method of treating depression in a subject in need thereof, the method comprising administering to the subject a therapeutically effective amount of dexmedetomidine or a pharmaceutically acceptable salt thereof in combination with a therapeutically effective amount of latrepirdine or a pharmaceutically acceptable salt thereof. The present disclosure provides a method of treating depression in a subject in need thereof, the method comprising administering oromucosally to the subject a therapeutically effective amount of dexmedetomidine or a pharmaceutically acceptable salt thereof in combination with a therapeutically effective amount of latrepirdine or a pharmaceutically acceptable salt thereof.

In embodiments, the present disclosure provides a method of treating depression in a subject in need thereof, the method comprising administering oromucosally to the subject about 1 mg to about 100 mg of latrepirdine or a pharmaceutically acceptable salt thereof and about 10 micrograms to about 300 micrograms of dexmedetomidine or a pharmaceutically acceptable salt thereof. In embodiments, the improvement in depressive symptoms is observed as measured by HAM-D-17 depression subscale. In embodiments, the subject has a HAM-D-17 total score ≥18 at the start of treatment.

In embodiments, the present disclosure provides a method of reducing score on HDRS

scale in a human subject suffering from depression comprising administering oromucosally effective amount of latrepirdine or a pharmaceutically acceptable salt thereof. In embodiments, the present disclosure provides a method of reducing score on HDRS scale in a human subject suffering from depression comprising administering oromucosally effective amount of dexmedetomidine or a pharmaceutically acceptable salt thereof.

In embodiments, the present disclosure provides a method of reducing score on HDRS

scale in a human subject suffering from depression comprising administering oromucosally effective amounts of dexmedetomidine in combination with latrepirdine or pharmaceutically acceptable salts thereof.

In embodiments, the present disclosure provides a method of reducing score on MADRS

scale in a human subject suffering from depression comprising administering oromucosally effective amount of latrepirdine or a pharmaceutically acceptable salt thereof. In embodiments, the present disclosure provides a method of reducing score on MADRS scale in a human subject suffering from depression comprising administering oromucosally effective amount of dexmedetomidine or a pharmaceutically acceptable salt thereof. In embodiments, the present disclosure provides a method of reducing score on MADRS scale in a human subject suffering from depression comprising administering oromucosally effective amounts of dexmedetomidine in combination with latrepirdine or pharmaceutically acceptable salts thereof.

In embodiments, the depression is moderate or severe. In embodiments, the depression is major depression, bipolar disorder or mixed depression.

In embodiments, the combination comprising latrepirdine and dexmedetomidine or salts thereof is administered once daily, twice daily, thrice daily or four times, five times, six times a day, preferably once, twice or thrice daily.

In embodiments, the combination comprising latrepirdine and dexmedetomidine or salts thereof is administered for at least 3 days, at least 5 days, at least 7 days, at least 10 days, at least 15 days, at least 30 days, at least 60 days, at least 90 days, at least 180 days, at least 365 days, or longer.

In embodiments, the present disclosure provides a synergistic combination comprising:
(i) a therapeutically effective amount of latrepirdine or a pharmaceutically acceptable salt thereof and
(ii) a therapeutically effective amount of dexmedetomidine or a pharmaceutically acceptable salt thereof, for the treatment of depression in a subject in need thereof.

In embodiments, the present disclosure provides a method of treating psychosis in a subject in need thereof, comprising administering a therapeutically effective amount of latrepirdine or a pharmaceutically acceptable salt thereof to the subject.

In embodiments, the present disclosure provides a method of treating psychosis in a subject in need thereof, comprising administering oromucosally (e.g. sublingually, buccally, or gingivally) a therapeutically effective amount of latrepirdine or a pharmaceutically acceptable salt thereof to the subject.

In embodiments, the present disclosure provides a method of treating psychosis in a subject in need thereof, the method comprising administering to the subject a therapeutically effective amount of dexmedetomidine or a pharmaceutically acceptable salt thereof in combination with a therapeutically effective amount of latrepirdine or a pharmaceutically acceptable salt thereof.

In embodiments, the present disclosure provides a method of treating psychosis in a subject in need thereof, the method comprising administering oromucosally e.g. sublingually, buccally, or gingivally) to the subject a therapeutically effective amount of dexmedetomidine or a pharmaceutically acceptable salt thereof in combination with a therapeutically effective amount of latrepirdine or a pharmaceutically acceptable salt thereof.

In embodiments, the treatment is effective without causing significant sedation.

In embodiments, the treatment is effective without experiencing clinically significant cardiovascular effects. In embodiments, the severity of psychosis in the subject is assessed using PANS S scale.

In embodiments, the present disclosure provides a method of achieving a PANS S score reduction in psychosis for a sustained period of time in a subject comprising administering to the subject a therapeutically effective amount of latrepirdine or a pharmaceutically acceptable salt thereof.

In embodiments, the present disclosure provides a method of achieving a PANSS score reduction in psychosis for a sustained period of time in a subject comprising administering oromucosally to the subject a therapeutically effective amount of latrepirdine or a pharmaceutically acceptable salt thereof.

In embodiments, the present disclosure provides a method of achieving a PANS S score reduction in psychosis for a sustained period of time in a subject comprising administering to the subject a therapeutically effective amount of dexmedetomidine or a pharmaceutically acceptable salt thereof in combination with a therapeutically effective amount of latrepirdine or a pharmaceutically acceptable salt thereof.

In embodiments, the present disclosure provides a method of achieving a PANS S score reduction in psychosis for a sustained period of time in a subject comprising administering oromucosally to the subject a therapeutically effective amount of dexmedetomidine or a pharmaceutically acceptable salt thereof in combination with a therapeutically effective amount of latrepirdine or a pharmaceutically acceptable salt thereof.

In embodiments, the PANSS score reduction is at least about 20% to about 50% from baseline score. In embodiments, the PANSS score reduction is about 25% from baseline score.

In embodiments, the PANSS total score reduction is about 30% from baseline score. In embodiments, the PANSS total score reduction is about 35% points from baseline score. In embodiments, the PANS S total score reduction is about 40% points from baseline score. In embodiments, the PANSS total score reduction is about 45% points from baseline score. In embodiments, the PANSS total score reduction is about 50% points from baseline score

In embodiments, the psychosis is acute. In embodiments, the psychosis is chronic. In embodiments, the subject is agitated. In embodiments, the subject is non-agitated.

In embodiments, the psychosis is associated with a neuropsychiatric disorder selected from the group consisting of schizophrenia, schizoaffective disorder, depression, dementia and bipolar disorder or another related neuropsychiatric disorder. In embodiments, the psychosis is associated with neurodegenerative disorders.

In embodiments, the psychosis is associated with diseased condition such as substance abuse disorders (e.g., alcohol, opioid and other substance withdrawal).

In embodiments, the psychosis is acute. In embodiments, the psychosis is chronic. In embodiments, the psychosis is a single episode. In embodiments, the psychosis is recurring or includes recurrent episodes. In embodiments, the acute psychosis is associated with acute psychotic episodes and/or mixed episodes.

In embodiments, the dosage form disintegrates within about 1 second to about 10 minutes upon contact with an oral mucosa. In embodiments, the dosage form disintegrates in more than 1 minute upon contact with an oral mucosa. In embodiments, the dosage form disintegrates within about 5 seconds to about 2 minutes upon contact with an oral mucosa. In embodiments, the dosage form disintegrates within about 5 seconds to about 5 minutes upon contact with an oral mucosa. In embodiments, the dosage form disintegrates within about 5 seconds to about 10 minutes upon contact with an oral mucosa. In embodiments, the dosage form disintegrates in less than 60 seconds.

In embodiments, the dosage form is an oromucosal dosage form comprising:
(i) a therapeutically effective amount of latrepirdine or a pharmaceutically acceptable salt thereof;
(ii) one or more mucoadhesive agents and
(iii) one or more pharmaceutically acceptable excipients or carriers;
wherein the dosage form disintegrates within about 5 seconds to about 10 minutes upon contact with an oral mucosa.

In embodiments, the dosage form is an oromucosal dosage form comprising:
(i) a therapeutically effective amount of dexmedetomidine or a pharmaceutically acceptable salt thereof;
(ii) one or more mucoadhesive agents and
(iii) one or more pharmaceutically acceptable excipients or carriers;
wherein the dosage form disintegrates within about 5 seconds to about 10 minutes upon contact with an oral mucosa.

In embodiments, the dosage form is an oromucosal dosage form comprising:
(i) a therapeutically effective amount of latrepirdine or a pharmaceutically acceptable salt thereof;
(ii) a therapeutically effective amount of dexmedetomidine or a pharmaceutically acceptable salt thereof;
(iii) one or more mucoadhesive agents and
(iv) one or more pharmaceutically acceptable excipients or carriers;
wherein the dosage form disintegrates within about 5 seconds to about 10 minutes upon contact with an oral mucosa.

In embodiments, the oromucosal dosage form is a tablet, capsules, patch, film, sachet, wafer, powder, minitablet, pellet, paste, gel, ointment, cream, drops, liquid (e.g., solution, suspension or emulsion), spray, microspheres or nanospheres which can be formulated in accordance with methods that are standard in the art.

In embodiments, the dosage form is an oromucosal tablet for sublingual or buccal, or gingival administration. In embodiments, the dosage form is lyophilized (or freeze-dried).

In embodiments, the dosage form is a lyophilized sublingual or buccal or gingival tablet comprising:
(i) a therapeutically effective amount of latrepirdine or a pharmaceutically acceptable salt thereof;
(ii) sodium alginate;
(iii) croscarmellose sodium or sodium starch glycolate;
(iv) sucralose;
(v) magnesium stearate and/or silicon dioxide;
(vi) lactose or mannitol and
(vii) optionally other pharmaceutical acceptable excipients;
wherein the dosage form disintegrates within about 5 seconds to about 10 minutes upon contact with an oral mucosa.

In embodiments, the dosage form is a lyophilized sublingual or buccal or gingival tablet comprising:
(i) a therapeutically effective amount of latrepirdine or a pharmaceutically acceptable salt thereof;
(ii) carbomer;
(iii) croscarmellose sodium or sodium starch glycolate;
(iv) sucralose;
(v) magnesium stearate and/or silicon dioxide;
(vi) lactose or mannitol and
(vii) optionally other pharmaceutical acceptable excipients; wherein the dosage form disintegrates within about 5 seconds to about 10 minutes upon contact with an oral mucosa.

In embodiments, the dosage form is a lyophilized sublingual or buccal or gingival tablet comprising:
(i) a therapeutically effective amount of latrepirdine or a pharmaceutically acceptable salt thereof;
(ii) xanthan gum;
(iii) croscarmellose sodium or sodium starch glycolate;
(iv) sucralose;
(v) magnesium stearate and/or silicon dioxide;
(vi) lactose or mannitol; and
(vii) optionally other pharmaceutical acceptable excipients;
(viii) wherein the dosage form disintegrates within about 5 seconds to about 10 minutes upon contact with an oral mucosa.

In embodiments, the dosage form is a lyophilized sublingual or buccal or gingival tablet comprising:
(i) a therapeutically effective amount of latrepirdine or a pharmaceutically acceptable salt thereof;
(ii) a therapeutically effective amount of dexmedetomidine or a salt thereof;
(iii) sodium alginate, xanthan gum, carbomer, hydroxypropyl cellulose, hydroxypropyl methylcellulose or polyethylene oxide;
(iv) croscarmellose sodium or sodium starch glycolate;
(v) sucralose;
(vi) magnesium stearate and/or silicon dioxide;
(vii) lactose or mannitol and
(viii) optionally other pharmaceutical acceptable excipients;
wherein the dosage form disintegrates within about 5 seconds to about 10 minutes upon contact with an oral mucosa.

In embodiments, the dexmedetomidine and latrepirdine are provided as a single dosage form as an oromucosal tablet for the treatment of agitation comprising a therapeutically effective amounts of dexmedetomidine and latrepirdine or pharmaceutically acceptable salts thereof. In embodiments, the agitation is caused by noradrenergic hyperarousal. In embodiments, the agitation is treated without also inducing significant sedation.

In embodiments, the dexmedetomidine and latrepirdine are provided as a single dosage form as an oromucosal tablet for the treatment of depression, comprising a therapeutically effective amounts of dexmedetomidine and latrepirdine or pharmaceutically acceptable salts thereof.

In embodiments, the dexmedetomidine and latrepirdine are provided as two separate dosage forms as oromucosal tablets for treatment of agitation, one comprising a therapeutically effective amount of dexmedetomidine or a pharmaceutically acceptable salt thereof, and the other comprising a therapeutically effective amount of latrepirdine or a pharmaceutically acceptable salt thereof. In embodiments, the active agents dexmedetomidine and latrepirdine are administered concurrently to the subject in need thereof. In embodiments, the active agents dexmedetomidine and latrepirdine are administered sequentially to the subject in need thereof. In embodiments, the agitation is caused by noradrenergic hyperarousal. In embodiments, the agitation is treated without also inducing significant sedation.

In embodiments, the dexmedetomidine and latrepirdine are provided as two separate dosage forms as oromucosal tablets for the treatment of depression in a subject, one comprising a therapeutically effective amount of dexmedetomidine or a pharmaceutically acceptable salt thereof, and the other comprising a therapeutically effective amount of latrepirdine or a pharmaceutically acceptable salt thereof. In embodiments, the active agents dexmedetomidine and latrepirdine are administered concurrently to the subject in need thereof. In embodiments, the active agents dexmedetomidine and latrepirdine are administered sequentially to the subject in need thereof.

In embodiments, the active agents, dexmedetomidine and latrepirdine, or pharmaceutically acceptable salt thereof, are administered concurrently (e.g. single dosage form or two separate dosage forms) to the subject for a specific period of time (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days or so on) followed by single agent administration of latrepirdine to the subject for a specific period of time (e.g., about 1, 2, 3, 4, 5, 6, 7, 8, 9, 10, 11, 12 months or so on). In embodiments, dexmedetomidine or salt thereof is administered at least 1 hour before administration of latrepirdine so that symptoms are relieved as early as possible. In embodiments, dexmedetomidine is administered at least 0.5 hour before the administration of latrepirdine so that symptoms are relieved as early as possible. In embodiments, dexmedetomidine is administered at least 0.25 hours before the administration of latrepirdine so that symptoms are relieved as early as possible. In embodiments, dexmedetomidine is administered simultaneously with latrepirdine so that symptoms are relieved as early as possible.

In embodiments, the active agents, dexmedetomidine and latrepirdine, or pharmaceutically acceptable salt thereof, are administered intermittently (e.g. single dosage form or two separate dosage forms) to the subject for a specific period of time (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days, 1 month, 2 months, 3 months or so on) followed by a rest period ((e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days or so on) and then dosing period (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days, 1 month, 2 months, 3 months or so on).

In embodiments, the active agents are sequentially administered separated by an appropriate period of time such as about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 20 minutes, about 25 minutes, about 30 minutes, about 35 minutes, about 40 minutes, about 45 minutes, about 50 minutes, about 55 minutes, about 60 minutes (1 hour), about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours, including all ranges and values in between. In embodiments, the active agents are administered simultaneously at the same time or within a short period of time, usually less than about 60 minutes (i.e., about 1 hour), preferably about 45 minutes, more preferably about 15 minutes. In embodiments, the active agents are administered simultaneously at the same time or within a short period of time, usually less than about 60 minutes (i.e., about 1 hour), preferably about 45 minutes, more preferably about 15 minutes.

In embodiments, the present disclosure provides an individual unit oromucosal lyophilized tablet dosage form provided as a kit comprising:
(i) a therapeutic effective amount of dexmedetomidine or a pharmaceutically acceptable salt thereof;
(ii) a therapeutic effective amount of latrepirdine or a pharmaceutically acceptable salt thereof and
(iii) instructions for the administration of (i) and (ii) to a subject in need thereof.

In embodiments, the present disclosure provides a two-unit dosage form provided as a kit comprising:
(i) a first oromucosal lyophilized tablet dosage form comprising a therapeutic amount of dexmedetomidine or a pharmaceutically acceptable salt thereof;
(ii) a second oromucosal lyophilized tablet dosage form comprising a therapeutic amount of latrepirdine or a pharmaceutically acceptable salt thereof and
(iii) instructions for the simultaneous, sequential or separate administration of (i) and (ii) to a subject in need thereof.

### BRIEF DESCRIPTION OF THE DRAWINGS

FIG. 1: depicts an elevated plus maze apparatus. The maze consists of two closed arms and two open arms forming a cross, with a square central platform according to example 2.

FIG. 2: depicts the study design and administration time points of various drugs according to example 2.

FIG. 3: depicts the time (in seconds) Wistar rats spent in the open arm of the elevated plus maze after administration of latrepirdine dihydrochloride hydrate (3 mg/kg and 10 mg/kg) as compared to dextromethorphan (10 mg/kg) and fluvoxamine maleate (10 mg/kg) to the yohimbine hydrochloride (2.5 mg/kg) administered rats according to example 2.

FIG. 4: depicts the number of times Wistar rats entered the open arms of the elevated plus maze after administration of latrepirdine (3 mg/kg and 10 mg/kg) as compared to dextromethorphan hydrobromide (10 mg/kg) and fluvoxamine maleate (10 mg/kg) to the yohimbine hydrochloride (2.5 mg/kg) administered rats according to example 2.

FIG. 5: depicts the time (in seconds) Wistar rats spent in the open arm of the elevated plus maze after administration of latrepirdine dihydrochloride hydrate (0.1 mg/kg, 0.3 mg/kg, 1 mg/kg and 3 mg/kg) to the yohimbine hydrochloride (2.5 mg/kg) administered rats with latrepirdine dihydrochloride hydrate (3 mg/kg+saline) as per se treatment according to example 3.

FIG. 6: depicts the number of times Wistar rats entered the open arms of the elevated plus maze after administration of latrepirdine dihydrochloride hydrate (0.1 mg/kg, 0.3 mg/kg, 1 mg/kg and 3 mg/kg) to the yohimbine hydrochloride (2.5 mg/kg) administered rats with latrepirdine dihydrochloride hydrate (3 mg/kg+saline) as per se treatment according to example 3.

FIG. 7: depicts the effects on immobility (7A), swimming (7B), climbing (7C) and total active (7D) behaviors (in seconds) during the 5-minutes FST test in Sprague-Dawley (SD) rats in dark phase after administration of dexmedetomidine hydrochloride (1 μg/kg and 5 μg/kg), latrepirdine dihydrochloride hydrate (1 mg/kg) and combination of dexmedetomidine hydrochloride and latrepirdine dihydrochloride hydrate (1 μg/kg+1 mg/kg and 5 μg/kg+1 mg/kg, respectively) according to example 4.

FIGS. 8A & 8B: depicts the effect of dexmedetomidine hydrochloride (4 μg/kg and 10 μg/kg) and latrepirdine dihydrochloride hydrate (0.3 mg/kg, 1 mg/kg, 3 mg/kg and 10 mg/kg) on duration of attack (in seconds) in Swiss albino mice in resident intruder task according to example 5.

FIGS. 9A & 9B: depicts the effect of combinations of dexmedetomidine hydrochloride and latrepirdine dihydrochloride hydrate (4 μg/kg+0.3 mg/kg, 4 μg/kg+1 mg/kg, 4 μg/kg+3 mg/kg, 4 μg/kg+10 mg/kg and 10 μg/kg+1 mg/kg, respectively) on duration of attack (in seconds) in Swiss albino mice in resident intruder task according to example 5.

FIG. 10: depicts the effect of dexmedetomidine hydrochloride (4 μg/kg and 10 μg/kg), latrepirdine dihydrochloride hydrate (0.3 mg/kg, 1 mg/kg, 3 mg/kg and 10 mg/kg) and combinations of dexmedetomidine hydrochloride and latrepirdine dihydrochloride hydrate (4 μg/kg+0.3 mg/kg, 4 μg/kg+1 mg/kg, 4 μg/kg+3 mg/kg, 4 μg/kg+10 mg/kg and 10 μg/kg+1 mg/kg, respectively) on duration of attack (in seconds) in Swiss albino mice in resident intruder task according to example 5.

FIG. 11: shows the SMARTCUBE signatures of dexmedetomidine hydrochloride, latrepirdine and combination of dexmedetomidine hydrochloride and latrepirdine for antipsychotic behavior according to example 6.

FIG. 12: depicts the effect of dexmedetomidine hydrochloride (4 μg/kg, 10 μg/kg, 20 μg/kg and 30 μg/kg), latrepirdine dihydrochloride hydrate (1 mg/kg, 3 mg/kg and 10 mg/kg) and combinations of dexmedetomidine hydrochloride and latrepirdine dihydrochloride hydrate (4 μg/kg+10 mg/kg and 10 μg/kg+1 mg/kg, respectively) on distance traveled (in centimeters) in Swiss albino mice in open field test according to example 7.

### DETAILED DESCRIPTION

Yohimbine is an alpha-2 adrenergic antagonist and is useful to test the norepinephrine-mediated hyper-arousal pathway. As described herein, yohimbine was used to induce sympathetic hyper-arousal in rodents and three different classes of drugs were tested to determine their ability to reduce hyper-arousal. Surprisingly, only latrepirdine (Dimebon), a drug with complex pharmacology, was able to reduce hyper-arousal. Latrepirdine is an orally active, small molecule compound that operates through multiple mechanisms of action and works through complex mechanism.

Administration of an alpha-2 adrenergic receptor agonist or a pharmaceutically acceptable salt thereof is a particularly effective and safe intervention for the treatment of agitation. Dexmedetomidine is an alpha-2 adrenergic agonist and is reported to have anti-agitational effects when administered intravenously during surgical procedures and intensive care unit (ICU) setups.

The inventors of the present application have found that administration of latrepirdine either alone or in combination with dexmedetomidine provides a significantly improved outcomes in anti-agitation response along with other benefits over conventional treatment.

In a forced swim model in rodents, it was discovered that the administration of a combination comprising latrepirdine and dexmedetomidine provided significant increase in swimming behavior (correlated to anti-depressant effect) as compared to dexmedetomidine and latrepirdine administered individually, as described below in Example 4.

### ABBREVIATIONS

AD: Alzheimer's disease
AMPA: α-amino-3-hydroxy-5-methyl-4-isoxazolepropionic acid
ANOVA: Analysis of variance
CNS: Central nervous system
CT/CAT scan: Computed tomography scan
EPM: Elevated Plus Maze
FTD: Frontotemporal dementia
GABA: Gamma-aminobutyric Acid
5-HT: 5-Hydroxytryptamine
HDRS or (HAM-D): Hamilton Depression Rating Scale
ICU: Intensive care unit
IPD: In-Patient department
IM: Intramuscular
IP: Intraperitoneal
MRI: Magnetic resonance imaging
μg: Microgram
mg: Milligram
MADRS: Montgomery-Asberg Depression Rating Scale
MW: Molecular weight
NE: Nor-epinephrine
NMDA: N-methyl-D-aspartate
OPD: Out-patient department
PANSS: Positive and Negative Syndrome Scale
PTSD: Post-traumatic stress disorders
wt %: Weight percentage

### Definitions

It will be understood that the terminology used herein is for the purpose of describing embodiments only and is not intended to be limiting. As used in this specification, the singular forms “a”, “an” and “the” include plural referents unless the context clearly dictates otherwise.

Unless otherwise indicated, any reference to a compound herein, such as latrepirdine, dexmedetomidine by structure, name, or any other means, includes pharmaceutically acceptable salts; alternate solid forms, such as polymorphs, solvates, hydrates, etc.; tautomers; deuterium-modified compounds, such as deuterium modified latrepirdine or dexmedetomidine; or any chemical species that may rapidly convert to a compound described herein under conditions in which the compounds are used as described herein.

As used herein, “about” or “approximately,” when used in connection with a numerical variable, generally refers to the value of the variable and to all values of the variable that are within the experimental error (e.g., within the 95% confidence interval for the mean) or within ±10% of the indicated value, whichever is greater.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “a” or “an” refers to one or more of that entity. In addition, reference to “an agent” by the indefinite article “a” or “an” does not necessarily exclude the possibility that more than one of the agents are present.

As used herein, the term “comprise” as is used in this description and in the claims and its conjugations are used in its non-limiting sense to mean that items following the word are included, but items not specifically mentioned are not excluded. The present disclosure may suitably “comprise”, “consist of”, or “consist essentially of”, the steps, elements, and/or reagents described in the claims.

As used herein, the term “subject” preferably refers to a human patient. In embodiments, the subject can be any animal, including non-human mammals, such as mice, rats, other rodents, rabbits, dogs, cats, swine, cattle, sheep, horses, or primates.

As used herein, unless indicated otherwise, the terms “dosage form” “pharmaceutical composition”, “composition”, “formulation” and “composition of the disclosure,” are used interchangeably. Unless stated otherwise, the terms are meant to encompass, and are not limited to, dosage form containing drug substance i.e. dexmedetomidine or latrepirdine or both.

As used herein, the term “an effective amount” is interchangeable with “therapeutically effective dose,” or “therapeutically effective amount,” and refers to an amount sufficient to produce the desired effect. An effective amount is sufficient to cause an improvement in a clinically significant condition of the subject. An effective amount can be administered in one or more administrations, applications, or dosages.

As used herein, “pharmaceutically acceptable salt” refers to a salt known to be non-toxic and commonly used in the pharmaceutical literature. Typical inorganic acids used to form such salts include hydrochloric, hydrobromic, hydroiodic, nitric, sulfuric, phosphoric, hypophosphoric, and the like. Salts derived from organic acids, such as aliphatic mono and dicarboxylic acids, phenyl substituted alkanoic acids, hydroxyalkanoic and hydroxyl alkandioic acids, aromatic acids, aliphatic and aromatic sulfonic acids may also be used. A preferred salt is hydrochloride (or dihydrochloride) salt.

The term “treatment” is used herein to mean to relieve or alleviate at least one symptom of a disease in a subject. For example, in relation to behavioral disorders, the term “treatment” may mean to relieve or alleviate agitation and any combination of its manifestations in an agitated subject (e.g. pacing, rocking, gesturing, pointing fingers, restlessness, performing repetitious mannerisms, yelling, speaking in an excessively loud voice, using profanity, screaming, shouting, grabbing, shoving, pushing, resisting, hitting others, kicking objects or people, scratching, biting, throwing objects, hitting self, slamming doors, tearing things, destroying property, etc.). Treatment may be measured as a reduction level by at least 10% or higher, preferably 20% or higher, more preferably 40% or higher, even more preferably 60% or higher, still more preferably 80% or higher, and 90% or higher, as compared to a control. For example, in the context of agitation, the skilled artisan will understand that treatment can be measured in terms of well-known agitation scales, such as PEC score, CGI-I, and ACES (each of which is described in detail in WO/2020/006119, which is incorporated by reference in its entirety for all purposes). As an example, when agitation is treated in a patient, the patient may experience at least a 10%, 20%, 30%, 40%, 50%, 60%, 70%, 80%, 90%, or 100% or greater reduction in PEC total score from baseline (e.g. measured at 2 hours post-dose). Similarly, a treated patient may be measured on the CGI-I scale and may refer to a patient that has a score of 1 or 2 (e.g. measured at 1, 2, or 4 hours post-dose) or the Agitation-Calmness Evaluation Scale (ACES) scale and may refer to a patient that has a score of 3, 4, 5, 6, or 7.

The term “oromucosal delivery” or “oromucosal administration” and the like means administration to the oral mucosa. It includes delivery across any tissue of the mouth, pharynx, larynx, trachea, or upper gastrointestinal tract, particularly including the sublingual, buccal, gingival and palatal mucosal tissues.

The term “sublingual” means “under the tongue” and refers to a method of administering substances via the mouth in such a way that the substances are rapidly absorbed via the blood vessels under the tongue rather than via the digestive tract. Sublingual absorption occurs through the highly vascularized sublingual mucosa, which allows a substance direct access to the blood circulation, thereby providing for direct systemic administration independent of gastrointestinal influences and avoiding undesirable first-pass hepatic metabolism. Accordingly, by administering the dosage forms of the present disclosure sublingually, the total amount of latrepirdine and/or dexmedetomidine may be reduced, thereby reducing the likelihood of deleterious side effects and providing a cost benefit to the manufacturer.

The term “buccal” means administration of the dosage form against the gum and the inner lip or cheek. Oromucosal absorption occurs through the highly vascularized transmucosal mucosa, which allows a substance direct access to the blood circulation, thereby providing for direct systemic administration independent of gastrointestinal influences and avoiding undesirable first-pass hepatic metabolism.

The term “disintegrate” refers to the breaking up of or loss of structural cohesion of the constituent particles comprising a solid formulation. This can occur in a number of different ways including breaking into smaller pieces and ultimately, fine and large particulates or, alternatively, eroding from the outside in until the dosage form has disappeared

The terms “oral dosage form”, “oromucosal dosage form,” and “oral transmucosal dosage form,” may be used interchangeably herein and refer to a dosage form for use in practicing the present disclosure, which comprises a drug formulation as described herein. The oral dosage form is typically a sublingual or buccal dosage form, but in some cases other oral transmucosal routes may be employed. The disclosure relies upon such oral dosage forms to provide sustained delivery of drugs across the oral mucosa; by controlling the formulation design immediate, intermediate and sustained release of drugs can be achieved, as described below. The dosage form comprises active ingredients (dexmedetomidine and/or latrepirdine) and one or more mucoadhesives that provide for adherence to the mucosa of the mouth of a patient, and other carriers and excipients described in more detail herein.

The term “orally disintegrating tablet” (ODT) refers to an oral dosage form composed of a tablet that is designed to disintegrate in the oral cavity without need for chewing or swallowing with liquids. Orally disintegrating tablets may have the characteristics set forth by the U.S. Food & Drug Administration in Guidance for Industry: Orally Disintegrating Tablets (Dept. of Health and Human Services, U.S. FDA Center for Drug Evaluation and Research, December 2008). The rate of disintegration of the solid pharmaceutical compositions of the present disclosure can be measured using various in vitro test methods, for example the USP <701> Disintegration Test. As explained in the foregoing section of the U.S Pharmacopoeia, the USP Disintegration Test is conducted by placing the dosage form to be tested in a basket rack assembly, immersing the assembly in a specified fluid at a temperature between 35° C. and 39° C. for a given time period, and raising and lowering the basket in the immersion fluid through a distance of about 5.5 cm at a frequency of about 30 cycles per minute. The dosage forms are visually inspected at specified times for complete disintegration, defined in Section 701 of USP 24-NF 19 as the state in which any residue of the dosage form remaining in the basket rack of the test apparatus is a “soft mass having no palpably firm core.” As such, it will be appreciated that the present dosage forms are optimal for disintegration in the mouth without the need to drink additional water. Adsorption may be through the oral mucosa.

The term “film” herein includes thin films, sheets and wafers, in any shape, including rectangular, square, or other desired shape. The film may be of any desired thickness and size, such that it can be conveniently placed sub-lingually in the patient. For example, the film may be a relatively thin film having a thickness of from about 20 micrometers to about 200 micrometers or may be a somewhat thicker film having a thickness of from about 20 micrometers to about 1000 micrometers. In embodiments, the film may be even thicker, e.g., having a thickness greater than about 30 millimeters.

The term “mucoadhesion” is used herein to refer to adhesion to mucosal membranes, such as those in the oral cavity. Mucoadhesion may be measured in “mucoadhesive strength” or “mucoadhesive peak force”.

The term “mucoadhesive” is a material that adheres to a mucosal tissue surface in-vivo. Such adhesion adherently localizes the dosage form onto the mucus membrane and requires the application of a force to separate the mucoadhesive material from the mucus membrane.

“Therapeutic” as used herein, may mean treatment and/or prophylaxis, depending on context.

The term “agitation”, as used herein, means a disorder characterized by symptoms of irritability, emotional outburst, impaired thinking, or excess motor and verbal activity that may occur due to either dysfunction of specific brain regions such as frontal lobes or due to dysfunction of neurotransmitter systems such as the noradrenergic system. In embodiments, the agitation may be caused by noradrenergic hyperarousal. In the present disclosure, an agitated subject may also exhibit aggression. The agitation may be acute or chronic. The agitation may be severe.

The term “acute agitation” means agitation that occurs rapidly and is severe and sudden in onset. Acute agitation may be associated with, for example, neurodegenerative disorders and neuropsychiatric disorders, although it may particularly exist in neuropsychiatric conditions. Acute agitation may lead to chronic agitation if it remains untreated.

The term “chronic agitation” means agitation developed over a long period of time and is less severe than acute agitation. Chronic agitation may be associated with, for example, neurodegenerative disorder and neuropsychiatric disorders, although it may particularly exist in neurodegenerative disorders.

The term “without significant sedation” or “without inducing significant sedation” and the like means that the patient experiences a level of sedation not greater than Level 3 on the Ramsay Sedation Scale. Level 3 means sedated but responds to commands. In embodiments, the dexmedetomidine may be dosed to achieve a Richmond Agitation Sedation Scale (RASS) of −1 (“light sedation”).

The term “neurodegenerative disorder” includes, but is not limited to, Alzheimer's disease, frontotemporal dementia (or Pick's disease), Dementia (e.g., Dementia with Lewy bodies, vascular dementia), posttraumatic stress disorder (PTSD), Parkinson's disease, vascular cognitive impairment, Huntington's disease, multiple sclerosis, Creutzfeldt-Jakob disease, multiple system atrophy, progressive supranuclear palsy.

The term “neuropsychiatric disorder” includes, but is not limited to, schizophrenia, bipolar illness (bipolar disorder, bipolar mania), depression, delirium.

The term “sundown syndrome” refers to a late-day circadian syndrome of increased confusion, agitation, and/or restlessness, generally in a patient with some form of dementia. Some subjects affected with sundown syndrome may have more than one disease or disorders. For example, a patient with sundown syndrome may suffer from dementia, Alzheimer's disease, or both. About 20-45% of Alzheimer type patients will experience some sort of sundowning confusion. Sundown syndrome also refers to confusion and/or agitation worsening in the late afternoon, evening, or as the sun goes down.

The term “behavioral and psychological symptoms” refer to agitation/aggression, delusions and/or hallucinations, aberrant motor behavior, aberrant vocalizations, anxiety, euphoria/elation, irritability, depression/dysphoria, apathy, disinhibition, sleep and night-time behavior change, and appetite and eating change.

The term “freeze-drying” or “lyophilization” refers to a process used in the creation of a stable preparation of a substance by freezing a fluid formulation containing the substance and substantially remove the frozen liquid under vacuum. The term “lyophilization” refers to the conventional, art-recognized procedure freeze-drying a composition. “Lyophilized” and “freeze-dried” are used herein as synonyms.

The term “pharmaceutically acceptable carrier” refers to a pharmacologically inert substance to be used as a carrier. As used herein, the phrase “carrier” and “excipients” are used interchangeably, except where otherwise clearly intended to have different meanings.

The term “unit dosage form” as used herein refers to a physically discrete units, suitable as unit dosages, each unit containing a predetermined quantity of active ingredient calculated to produce the desired therapeutic effect in association with the required pharmaceutical carrier.

It will be understood, however, that the total daily usage of the compositions of the present disclosure will be decided by the attending physician within the scope of sound medical judgment.

Within the meaning of the present disclosure, the term “conjoint administration” or “simultaneous administration” is used to refer to administration of dexmedetomidine or a pharmaceutically acceptable salt thereof and latrepirdine or a pharmaceutically acceptable salt thereof at the same time in separate formulations or as a combination formulation, i.e. in a single dosage form.

The term “sequential administration” refers to administration of the latrepirdine and dexmedetomidine one after the other, i.e. not at the same time, in two or more separate dosage forms. If the disclosed drug substances are administered sequentially, then typically administration of the last drug substance is begun an hour or less, generally 30 minutes or less, after administration of the first drug substance is begun.

As used herein “nanonization” means the process of reducing the particles to be in a range such that the average particle size is less than 1000 nanometers in size preferably less than 100 nanometers in size.

The phrase “spray drying” refers to processes involved in breaking up liquid mixtures into small droplets (atomization) and rapidly removing solvent from the mixture in a spray drying apparatus where there is a strong driving force for evaporation of solvent from the droplets. The phrase spray drying is used conventionally and broadly. Spray drying processes and spray drying equipment are described generally in Perry, Robert H., and Don W. Green (eds.), Perry's Chemical Engineers' Handbook, New York: McGraw-Hill, 2007 (8^{th} edition).

The term “sublimation” refers to the physical phase transition from a solid state directly to a vapor state. More specifically, sublimation is a process in which a substance goes from a solid to a gas without going through a liquid phase. Sublimation of a solution may be obtained through the freeze-drying process.

As used herein, the term “granulation” refers to the process of agglomerating powder particles into larger agglomerates (i.e. granules) that contain the active agent. The term “granulation” includes dry and wet granulation techniques. The term “wet granulation” refers to any process comprising the steps of addition of a water comprising liquid, preferably water to the powder starting materials, preferably kneading, and drying to yield a solid dosage form. The term “dry granulation” refers to any process that comprises compacting the powder, usually either by slugging or with a roller compactor, and preferably milling the compacted powder to obtain the granules. No liquid is employed for the dry granulation. The compacted granulate or compacted granules as disclosed herein are preferably prepared by dry granulation.

“Direct compression” refers to a process which involves blending the ingredients and then compressing into tablets.

I. Active Agents

Dexmedetomidine has the IUPAC name (+) 4-(S)-[1-(2,3-dimethylphenyl) ethyl]-1H-imidazole. As the monohydrochloride salt, it is predominantly used as a medication for the sedation of patients during treatment in an intensive care setting or to sedate patients prior to and/or during surgical and other procedures. Such medication is currently sold under the registered trade name “PRECEDEX®”.

Pharmaceutically acceptable salts of dexmedetomidine that may be used herein include generally any suitable salt that has been or may be approved by the US FDA or other appropriate foreign or domestic agency for administration to a human. Non-limiting examples of suitable pharmaceutically acceptable salts include salts of inorganic acids such as hydrochloric, hydrobromic, nitric, carbonic, monohydrocarbonic, phosphoric, monohydrogen phosphoric, dihydrogen phosphoric, sulfuric, hydrogen sulfuric, and hydroiodic acid. Other examples include salts derived from non-toxic organic acids, including acetic, propionic, isobutyric, maleic, malonic, benzoic, succinic, suberic, fumaric, lactic, mandelic, phthalic, benzenesulfonic, p-toluenesulfonic, citric, tartaric, and methanesulfonic acids, or combinations of these acid salts. Exemplary salts include dexmedetomidine hydrochloride, dexmedetomidine hydrobromide, dexmedetomidine sulfate, dexmedetomidine sulfonate, dexmedetomidine phosphate, dexmedetomidine nitrate, dexmedetomidine formate, dexmedetomidine citrate, dexmedetomidine tartrate, dexmedetomidine malate, dexmedetomidine benzoate, dexmedetomidine salicylate, dexmedetomidine ascorbate or the like. In embodiments, deuterated forms of dexmedetomidine or a pharmaceutically acceptable salt thereof may be included.

Latrepirdine (also known as Dimebon) has the IUPAC name of 2,8-dimethyl-5-[2-(6-methylpyridin-3-yl)ethyl]-3,4-dihydro-1H-pyrido [4,3-b]indole and should be understood herein to include any pharmaceutically acceptable form. By “pharmaceutically acceptable form” is meant any pharmaceutically acceptable form, including, solvates, hydrates, isomorphs, polymorphs, co-crystals, pseudomorphs, neutral forms, acid addition salt forms, and prodrugs. It may be present in a form of salts with pharmaceutically acceptable acids and in a form of quarternized derivatives. pharmaceutically acceptable base addition salts can be prepared from inorganic and/or organic bases.

The pharmaceutically acceptable acid addition salts of latrepirdine are prepared in a conventional manner by treating a solution or suspension of the free base with, for example, one or two chemical equivalents of a pharmaceutically acceptable acid. Illustrative of suitable acids are acetic, lactic, succinic, maleic, tartaric, citric, gluconic, ascorbic, mesylic, tosylic, benzoic, cinnamic, fumaric, nitric, sulfuric, phosphoric, hydrochloric, dihydrochloric, hydrobromic, hydroiodic, sulfamic, sulfonic such as methanesulfonic, benzenesulfonic, and related acids. In embodiments, latrepirdine is present as a free base. In embodiments, latrepirdine is present as latrepirdine dihydrochloride. In embodiments, latrepirdine is present as latrepirdine hydrochloride. In embodiments, latrepirdine is present as latrepirdine dihydrochloride dihydrate. In embodiments, latrepirdine is present as latrepirdine dihydrochloride hydrate.

II. Dosages

In embodiments, the dosage of dexmedetomidine or a pharmaceutically acceptable salt thereof administered may conveniently be in the range of between about 0.5 micrograms to about 300 micrograms. Examples of suitable dosages include: about 0.5 micrograms to about 280 micrograms, about 1 microgram to about 270 micrograms about 1 microgram to about 260 micrograms, about 1 microgram to about 250 micrograms, about 1 microgram to about 240 micrograms, about 1 microgram to about 230 micrograms, about 1 microgram to about 220 micrograms, about 1 microgram to about 210 micrograms, about 1 microgram to about 200 micrograms, about 1 microgram to about 190 micrograms, about 1 microgram to about 180 micrograms, about 1 microgram to about 170 micrograms, about 1 microgram to about 160 micrograms, about 1 microgram to about 150 micrograms, about 1 microgram to about 140 micrograms, about 1 microgram to about 130 micrograms, about 1 microgram to about 120 micrograms, about 1 microgram to about 110 micrograms, about 1 microgram to about 100 micrograms, about 3 micrograms to about 90 micrograms, about 3 micrograms to about 80 micrograms, about 3 micrograms to 70 micrograms, about 3 micrograms to about 60 micrograms, about 3 micrograms to 50 micrograms, about 3 micrograms to about 40 micrograms, about 3 micrograms to about 35 micrograms, about 5 micrograms to about 35 micrograms, about 10 micrograms to about 50 micrograms, about 10 micrograms to about 40 micrograms, about 10 micrograms to about 35 micrograms or about 15 micrograms to 35 micrograms. The dose may be administered one or more times a day, e.g. two times, three times, four times, five times or six times per day.

In embodiments, the per unit dose of dexmedetomidine or a pharmaceutically acceptable salt thereof is about 10 micrograms, about 15 micrograms, about 20 micrograms, about 25 micrograms, about 30 micrograms, about 35 micrograms, about 40 micrograms, about 45 micrograms, about 50 micrograms, about 55 micrograms, about 60 micrograms, about 65 micrograms, about 70 micrograms, about 75 micrograms, about 80 micrograms, about 85 micrograms, about 90 micrograms, about 95 micrograms, about 100 micrograms, about 105 micrograms, about 110 micrograms, about 115 micrograms, about 120 micrograms, about 125 micrograms, about 130 micrograms, about 135 micrograms, about 140 micrograms, about 145 micrograms, about 150 micrograms, about 155 micrograms, about 160 micrograms, about 165 micrograms, about 170 micrograms, about 175 micrograms, about 180 micrograms, about 185 micrograms, about 190 micrograms, about 195 micrograms, about 200 micrograms, about 205 micrograms, about 210 micrograms, about 215 micrograms, about 220 micrograms, about 225 micrograms, about 230 micrograms, about 235 micrograms, about 240 micrograms, about 245 micrograms about 250 micrograms, about 255 micrograms, about 260 micrograms, about 265 micrograms, about 270 micrograms, about 275 micrograms, about 280 micrograms, about 285 micrograms, about 290 micrograms, about 295 micrograms or about 300 micrograms, including all values and ranges in between.

Each unit may be administered to the subject one or more times per day, e.g. 1, 2, 3, 4, 5, or 6 times per day. In embodiments, each unit may be administered at an appropriate dosing interval (e.g. about 1 hour between doses) or can be administered concurrently.

An effective total daily dose may, for example, include one or more-unit doses, up to a total daily dose of about 1 mg of dexmedetomidine or a pharmaceutically acceptable salt thereof. In embodiments, the total daily dose of dexmedetomidine or a pharmaceutically acceptable salt thereof is about 0.5 micrograms to about 500 micrograms, e.g. a total daily dose of about 20 micrograms, about 25 micrograms, about 30 micrograms, about 35 micrograms, about 40 micrograms, about 45 micrograms, about 50 micrograms, about 55 micrograms, about 60 micrograms, about 65 micrograms, about 70 micrograms, about 75 micrograms, about 80 micrograms, about 85 micrograms, about 90 micrograms, about 95 micrograms, about 100 micrograms, about 110 micrograms, about 120 micrograms, about 130 micrograms, about 140 micrograms, about 150 micrograms, about 160 micrograms, about 170 micrograms, about 180 micrograms, about 190 micrograms, about 200 micrograms, about 210 micrograms, about 220 micrograms, about 230 micrograms, about 240 micrograms, about 250 micrograms, about 260 micrograms, about 270 micrograms, about 280 micrograms, about 290 micrograms, about 300 micrograms, about 310 micrograms, about 320 micrograms, about 330 micrograms, about 340 micrograms, about 350 micrograms, about 360 micrograms, about 370 micrograms, about 380 micrograms, about 390 micrograms, about 400 micrograms, about 410 micrograms, about 420 micrograms, about 430 micrograms, about 440 micrograms, about 450 micrograms, about 460 micrograms, about 470 micrograms, about 480 micrograms, about 490 micrograms or about 500 micrograms, including all ranges and values in between.

In embodiments, the daily dose of latrepirdine or a pharmaceutically acceptable salt thereof administered may conveniently be in the range of between about 0.5 mg to about 100 mg. Examples of suitable dosages include: about 0.5 mg to about 450 mg, about 0.5 mg to about 400 mg, about 0.5 mg to about 350 mg, about 0.5 mg to about 300 mg, about 0.5 mg to about 250 mg, about 0.5 mg to about 200 mg, about 0.5 mg to about 150 mg, about 0.5 mg to about 100 mg, about 1 mg to about 500 mg, about 1 mg to about 450 mg, about 1 mg to about 400 mg, about 1 mg to about 350 mg, about 1 mg to about 300 mg, about 1 mg to about 200 mg, about 1 mg to about 150 mg, about 1 mg to about 100 mg, about 2 mg to about 500 mg, about 2 mg to about 450 mg, about 2 mg to about 400 mg, about 2 mg to about 350 mg, about 2 mg to about 300 mg, about 2 mg to about 250 mg, about 2 mg to about 200 mg, about 2 mg to about 150 mg, about 2 mg to about 100 mg, about 3 mg to about 500 mg, about 3 mg to about 450 mg, about 3 mg to about 400 mg, about 3 mg to about 350 mg, about 3 mg to about 300 mg, about 3 mg to about 250 mg, about 3 mg to about 200 mg, about 3 mg to about 150 mg, about 3 mg to about 100 mg, about 4 mg to about 500 mg, about 4 mg to about 450 mg, about 4 mg to about 400 mg, about 4 mg to about 350 mg, about 4 mg to about 300 mg, about 4 mg to about 250 mg, about 4 mg to about 200 mg, about 4 mg to about 150 mg, about 4 mg to about 100 mg, about 5 mg to about 500 mg, about 5 mg to about 450 mg, about 5 mg to about 400 mg, about 5 mg to about 350 mg, about 5 mg to about 300 mg, about 5 mg to about 250 mg, about 5 mg to about 200 mg, about 5 mg to about 150 mg, about 5 mg to about 100 mg, about 5 mg to about 90 mg, about 5 mg to about 80 mg, about 5 mg to about 70 mg, about 5 mg to about 60 mg, about 5 mg to about 50 mg, about 5 mg to about 40 mg, about 5 mg to about 30 mg, about 5 mg to about 20 mg or about 5 mg to about 10 mg. In embodiments, the daily dose of latrepirdine or a pharmaceutically acceptable salt thereof administered may conveniently be in the range of about 5 mg to about 60 mg, for example about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, or about 60 mg, including all values and ranges in between.

In embodiments, the per unit dose of latrepirdine is about 100 mg, about 95 mg, about 90 mg, about 85 mg, about 80 mg, about 75 mg, about 70 mg, about 65 mg, about 60 mg, about 55 mg, about 50 mg, about 45 mg, about 40 mg, about 35 mg, about 30 mg, about 25 mg, about 20 mg, about 15 mg, about 10 mg, about 8 mg, about 7 mg, about 6 mg, about 5 mg, about 4 mg, about 3 mg, about 2 mg, about 1 mg, about 0.5 mg or about 0.1 mg, including all ranges and values in between.

The exemplary dosages of dexmedetomidine or a pharmaceutically acceptable salt thereof (e.g. hydrochloride salt) and latrepirdine or a pharmaceutically acceptable salt thereof (e.g. dihydrochloride salt) to be administered to a particular patient, will depend on the type and extent of the condition, the overall health status of the particular patient, the particular form of dexmedetomidine and latrepirdine or pharmaceutically acceptable salts thereof being administered, and the particular formulation used to treat the patient.

III. Dosage Forms

In embodiments, the present disclosure provides an oromucosal dosage form comprising latrepirdine or a pharmaceutically acceptable salt thereof, one or more mucoadhesive agents and one or more pharmaceutically acceptable excipients or carriers.

In embodiments, the dosage forms described herein disintegrate within about 5 second to about 10 minutes upon contact with an oral mucosa, e.g. about 5 seconds to about 10 minutes, about 5 seconds to about 5 minutes, about 5 seconds to about 1 minute, about 5 seconds to about 30 seconds, about 5 seconds to about 10 seconds, about 30 seconds to about 10 minutes, about 30 seconds to about 5 minutes, about 30 seconds to about 1 minute, about 1 minute to about 10 minutes, about 1 minute to about 5 minutes, about 1 minute to about 2 minutes, about 2 minutes to about 10 minutes, about 2 minutes to about 5 minutes, about 2 minutes to about 3 minutes, about 3 minutes to about 10 minutes, about 3 minutes to about 5 minutes, about 4 minutes to about 10 minutes, about 4 minutes to about 5 minutes, about 5 minutes to about 10 minutes, about 5 minutes to about 7 minutes, or about 7 minutes to about 10 minutes upon contact with an oral mucosa. In embodiments, the dosage form disintegrates in less than about 1 minute upon contact with an oral mucosa, e.g. about 5 seconds, about 10 seconds, about 15 seconds, about 20 seconds, about 25 seconds, about 30 seconds, about 35 seconds, about 40 seconds, about 45 seconds, about 50 seconds, about 55 seconds, or about 60 seconds, including all ranges and values in between. In embodiments, the dosage form does not disintegrate within 1 minute upon contact with an oral mucosa. In embodiments, the dosage form disintegrates in more than 1 minute upon contact with an oral mucosa, e.g. about 1 minute, about 2 minutes, about 3 minutes, about 4 minutes, about 5 minutes, about 6 minutes, about 7 minutes, about 8 minutes, about 9 minutes, or about 10 minutes. Thus, dosage forms produced in accordance with one of the embodiments of the present disclosure meet the disintegration time criteria of disintegration within about 5 seconds to about 10 minutes when tested by the <701> disintegration test method (see Guidance to Industry, herein incorporated by reference).

In embodiments, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98% or at least about 99% of the drug in a dosage form comprising a formulation of the disclosure is absorbed via the oral mucosa.

In embodiments, the dosage forms of the present disclosure possess sufficient mechanical strength to resist attrition/chipping during packaging in blisters and bottles, storage and transportation for commercial distribution and end use.

In embodiments, the dosage forms described herein effectively treat agitation in an agitated subject. For example, the dosage forms described herein effectively treat agitation in a subject as measured by PEC, CGI-I, and/or ACES. In embodiments, the dosage form effectively treats agitation in a subject without also inducing significant sedation. In embodiments, the subject is treated without experiencing clinically significant cardiovascular effects. In embodiments, the agitation is caused by noradrenergic hyperarousal. In embodiments, the dosage forms described herein effectively reduces noradrenergic hyperarousal.

In embodiments, the dosage forms described herein effectively treat depression in a subject. For example, the dosage forms described herein effectively treat depression in a subject as measured by HAM-D scale or MADRS scale. In embodiments, the present disclosure provides an oromucosal dosage form comprising dexmedetomidine or a pharmaceutically acceptable salt thereof (e.g. hydrochloride salt), one or more mucoadhesive agents and one or more pharmaceutically acceptable excipients or carriers to treat agitation. In embodiments, the dosage form disintegrates in less than about 1 minute upon contact with an oral mucosa. For example, the dosage form may disintegrate in about 5 seconds, about 10 seconds, about 15 seconds, about 20 seconds, about 25 seconds, about 30 seconds, about 35 seconds, about 40 seconds, about 45 seconds, about 50 seconds, about 55 seconds, or about 60 seconds, including all ranges and values in between. In embodiments, the dosage form disintegrates more than about 1 minute upon contact with an oral mucosa. In embodiments, the dosage form disintegrates in not less than about 1 minute upon contact with an oral mucosa, e.g. about 1 minute, about 2 minutes, about 3 minutes, about 4 minutes, about 5 minutes, about 6 minutes, about 7 minutes, about 8 minutes, about 9 minutes, or about 10 minutes. In embodiments, the dosage form effectively treats agitation in a subject. In embodiments, the dosage form effectively treats agitation in a subject without also inducing significant sedation. In embodiments, the agitation is caused by noradrenergic hyperarousal. In embodiments, the dosage forms described herein effectively treats depression in a subject.

In embodiments, the present disclosure provides an oromucosal dosage form comprising dexmedetomidine or a pharmaceutically acceptable salt thereof and latrepirdine or a pharmaceutically acceptable salt thereof, one or more mucoadhesive agents and one or more pharmaceutically acceptable excipients or carriers to treat agitation. In embodiments, the dosage form disintegrates in less than about 1 minute upon contact with an oral mucosa. For example, the dosage form may disintegrate in about 5 seconds, about 10 seconds, about 15 seconds, about 20 seconds, about 25 seconds, about 30 seconds, about 35 seconds, about 40 seconds, about 45 seconds, about 50 seconds, about 55 seconds, or about 60 seconds upon contact with an oral mucosa. In embodiments, the dosage form disintegrates in not less than about 1 minute upon contact with an oral mucosa. For example, the dosage form may disintegrate in about 1 minute, about 2 minutes, about 3 minutes, about 4 minutes, about 5 minutes, about 6 minutes, about 7 minutes, about 8 minutes, about 9 minutes, or about 10 minutes. In embodiments, the dosage form effectively treats agitation in a subject. In embodiments, the dosage form effectively treats agitation in a subject without also inducing significant sedation. In embodiments, the agitation is caused by noradrenergic hyperarousal. In embodiments, the dosage forms described herein effectively treats depression in a subject.

In embodiments, the present disclosure provides a first oromucosal dosage form comprising dexmedetomidine or a pharmaceutically acceptable salt thereof and a second oromucosal dosage form comprising latrepirdine or a pharmaceutically acceptable salt thereof for conjoint administration either concurrently or sequentially to treat agitation.

In embodiments, the dosage form is administered for the treatment of agitation associated with neurodegenerative disorders, neuropsychiatric disorders and alcohol withdrawal or substance abuse withdrawal, including opioid withdrawal. In embodiments, the dosage form is administered for the treatment of agitation associated with an OPD/IPD procedure (e.g. MRI, CT or CAT scan, lumbar puncture, bone marrow aspiration/biopsy, tooth extraction or other dental procedures).

In embodiments, the present disclosure provides a first oromucosal dosage form comprising dexmedetomidine or a pharmaceutically acceptable salt thereof and a second oromucosal dosage form comprising latrepirdine or a pharmaceutically acceptable salt thereof for conjoint administration either concurrently or sequentially to treat depression in a subject.

In embodiments, the oromucosal (e.g. sublingual or buccal) dosage form of the disclosure is a tablet, capsule, disc, patch or film, sachet, wafer, powder, minitablet, pellet, paste, gel, ointment, cream, drops, liquid (solution, suspension or emulsion), spray, microspheres or nanospheres which can be formulated in accordance with methods that are standard in the art.

In embodiments, the dosage form is an oromucosal wafer. In embodiments, the wafer is lyophilized. In embodiments, the wafer disintegrates in less than about 1 minute upon contact with an oral mucosa. In embodiments, the wafer disintegrates in more than about 1 minute upon contact with an oral mucosa. In embodiments, the wafer comprises excipients such as hydroxypropyl cellulose, lactose, mannitol, glycine, and the like.

In embodiments, the dosage form is an oromucosal mini-tablet. In embodiments, the mini-tablet disintegrates in less than about 1 minute upon contact with an oral mucosa. In embodiments, the mini-tablet disintegrates in more than about 1 minute upon contact with an oral mucosa. In embodiments, the minitablet comprises excipients based on co-processed mannitol. In embodiments, the mini-tablet contains directly compressible excipients. In embodiments, the compressible excipient is in the form of a hydrate, and may be selected from organic compounds such as dextrose monohydrate, maltodextrin, lactose monohydrate, and dextrin, as well as inorganic compounds including dibasic calcium phosphate dihydrate, dibasic sodium phosphate dihydrate, dibasic sodium phosphate heptahydrate, dibasic sodium phosphate dodecahydrate, monobasic sodium phosphate monohydrate and monobasic sodium phosphate dihydrate. In embodiments, the rapidly disintegrating tablet portion includes a compressible excipient selected from the group consisting of isomalt, dextrose monohydrate, hydrogenated starch hydrolysate base, maltodextrin, lactose monohydrate, dextrin, mannitol, lactitol, sorbitol, xylitol, erythritol, sucrose, and lactose.

In embodiments, the oromucosal dosage form is in the form of a tablet or disc or packed powder.

In embodiments, the dosage form is a hard or compressed powdered sublingual or buccal tablet having a low grit component for an organoleptically pleasant mouth feel. In embodiments, the tablet (or particles thereof containing the active agent which can be compressed to form the tablet) comprises a protective outer coating e.g. any polymer conventionally used in the formation of microparticles and microcapsules. In embodiments, the dosage form is a sublingual (or buccal) tablet containing an effervescent agent. Sublingual compositions comprising effervescent agents are disclosed in U.S. Pat. No. 6,200,604, which is herein incorporated by reference in its entirety, for all purposes.

In embodiments, the oromucosal tablet conveniently includes the active ingredient within a matrix. In embodiments, the matrix is composed of, for example, at least one filler and/or a lubricant. Fillers include, for example, lactose or mannitol, and suitable lubricants include, but are not limited to, magnesium stearate, silicon dioxide and talc. The matrix may also include one or more of: a binder (e.g. povidone, a sugar or carboxymethylcellulose), a disintegrant (e.g. croscarmellose sodium, crospovidone or sodium starch glycolate), a sweetening agent (e.g. sucralose) and the like. The tablet may conveniently have a friability of about 2% or less and a hardness of about 15 to about 50 Newtons.

In embodiments, the oromucosal dosage form is in the form of a patch or film (e.g. thin film). The patch may have adhesive qualities to prevent movement or swallowing of the patch. Suitable film compositions comprising dexmedetomidine are disclosed in U.S. Pat. No. 10,792,246, which is incorporated herein by reference in its entirety for all purposes.

In embodiments, the oromucosal dosage form is in the form of a paste, gel or ointment. The viscosity of the paste, gel or ointment can be adjusted to allow for retention under the tongue or near gums or cheeks or upper lip.

In embodiments, the oromucosal dosage form is in a liquid form (e.g. as a solution, suspension or emulsion), and may be, for example, presented as a spray or as drops. In a particular embodiment of the disclosure, Latrepirdine and/or dexmedetomidine or pharmaceutically acceptable salts thereof are oromucosally administered in liquid form, e.g. in a flavored or unflavored physiological saline solution. The liquid dosage form may conveniently be administered under the tongue or near the gums or cheeks or upper lip as drops or as a spray. The solutions include the active ingredient together with a diluent such as water, normal saline, sodium chloride solution, or any other suitable solvent such as propylene glycol, glycerol, ethyl alcohol and so on. The diluent for the solution may particularly be physiological saline solution or water.

The spray dosage form of the present disclosure for oromucosal administration may include one or more pharmaceutically acceptable liquids (e.g. present in the amount of about 30% to about 99.99% by weight of the composition). Such liquids may be solvents, co-solvents, or non-solvents for dexmedetomidine and latrepirdine or pharmaceutically acceptable salts thereof. Examples of pharmaceutically acceptable liquids include water, ethanol, dimethyl sulfoxide, propylene glycol, polyethylene glycol, propylene carbonate, pharmaceutically acceptable oils (e.g., soybean, sunflower, peanut, peppermint etc.) and the like. The pharmaceutically acceptable liquid is selected either to dissolve the active pharmaceutical ingredient, to produce a stable, homogenous suspension or solution of it, or to form any combination of a suspension or solution. In addition to these ingredients, spray formulations may include one or more excipients such as viscosity modulating materials (e.g. polymers, sugars, sugar alcohols, gums, clays, silicas, and the like, such as polyvinylpyrrolidone (PVP); preservatives (e.g., ethanol, benzyl alcohol, propylparaben and methylparaben); flavoring agents (e.g. peppermint oil), sweeteners (e.g., sugars such as sucrose, glucose, dextrose, maltose, fructose, etc.), artificial sweeteners (e.g. saccharin, aspartame, acesulfame, sucralose), or sugar alcohols (e.g. mannitol, xylitol, lactitol, maltitol syrup); buffers and pH-adjusting agent (e.g., sodium hydroxide, citrate, and citric acid); coloring agents; fragrances, chelating agents (e.g., EDTA); UV absorbers and antifoam agents (e.g., low molecular weight alcohols, dimethicone). In addition to one or more of the aforementioned ingredients suitable for sublingual or buccal administration, sprays may include one or more excipients such as viscosity modulating materials (e.g. water soluble or water swellable polymers such as carbopol, hydroxypropyl cellulose, hydroxypropyl methyl cellulose, carboxymethyl cellulose).

Sprays may be made by mixing appropriate quantities of the foregoing ingredients in accordance with standard good manufacturing practices. Such excipients may be included in the formulation to improve patient or subject acceptance or taste, to improve bioavailability, to increase shelf-life, to reduce manufacturing and packaging costs, to comply with requirements of governmental regulatory agencies, and for other purposes. The relative amounts of each ingredient should not interfere with the desirable pharmacological and pharmacokinetic properties of the resulting formulation.

A patient may, in one embodiment, be treated by administering sublingually or buccally 1 to 2 actuations from a spray pump. An advantage of spray delivery is the ability to easily titrate patients by 1 or 2 doses as required by a single actuation.

Pump action sprays are characterized in requiring the application of external pressure for actuation, for example, external manual, mechanical or electrically initiated pressure. This is in contrast to pressurized systems, e.g., propellant-driven aerosol sprays, where actuation is typically achieved by controlled release of pressure e.g., by controlled opening of a valve.

The non-solid dosage forms of the disclosure may conveniently be administered by spraying, dripping, painting or squirting the composition under the tongue or near the gums or cheeks or upper lip.

In embodiments, the oromucosal tablet dosage form is prepared by lyophilization (or freeze-drying). A suspension comprising active agent(s) may be prepared with appropriate excipients and the active agent (latrepirdine/dexmedetomidine) suspension may be dispensed into blister packs and freeze-dried. An exemplary freeze-dried preparation platform that could be used for a latrepirdine and/or dexmedetomidine orally disintegrating table (ODT) is the ZYDIS® (Catalent, Somerset, NJ, USA) formulation. In particular, the excipients are blended and the active agents are separately milled to size and then mixed with the excipients. The solution/suspension then undergoes lyophilization by flash freezing and freeze drying. This aqueous solution/suspension must be chemically and morphologically stable throughout the dosing process. Gelatin may be used to give sufficient strength to the dosage form to prevent breakage during removal from packaging, but once placed in the mouth, the gelatin allows for immediate disintegration of the dosage form. Other alternatives such as fish gelatin and modified starches may be used. During processing, dosed solution/suspension is preferably frozen by passing through a gaseous medium. This serves to immobilize the solution/suspension rapidly, thereby improving the manufacture efficiency. Examples of oromucosal dosage forms includes orally disintegrating tablets disclosed in U.S. Pat. Nos. 6,509,040, 7,972,621, 1,054,8839, U.S. Pat. Nos. 9,775,819, 5,188,825, 5,631,023, 6,297,240, 6,413,549, 5,976,577, 6,156,339, 5,827,541, 5,729,958, 6,726,928, 9,192,580, 6,709,669, US Appl. Publication No. 20200138721, US Appl. Publication No. 20190276707, US Appl. Publication No. 20190314274, US Appl. Publication No. 20040156894, PCT Publication No. 1999038496, PCT Publication No. 2000044351, and US Appl. Publication No. 20090226522 and related patents/patent applications, which are herein incorporated by reference in their entirety, for all purposes.

Other methods of preparing oromucosal dosage forms such as ODTs may be used without limitation, and detailed description of general methods thereof have been disclosed, for example, in U.S. Pat. Nos. 5,837,287; 6,149,938; 6,212,791; 6,284,270; 6,316,029; 6,465,010; 6,471,992; 6,471,992; 6,814,978; 6,908,626; 6,908,626; 6,982,251; 7,282,217; 7,425,341; 7,939,105; 7,993,674; 8,048,449; 8,127,516; 8,158,152; 8,221,480; 8,256,233; 8,313,768, 5,039,540; 5,120,549; 5,330,763; 4,760,093; 4,760,094; and 4,767,789, which are herein incorporated by reference in their entirety, for all purposes.

Different technologies that may be used to prepare oromucosal dosage forms of the disclosure include but not limited to Flash Dose, Orasolv, durasolv, wowtab technology, Flash Tab Technology, Oraquick Technology, Quick-Dis Technology, Nanocrystal Technology, Shearform Technology, Ceform Technology, Pharmaburst technology, Frosta technology, Ziplet technology, Humidity treatment, Sintering, Lyoc Technolog, Quicksolv Technology, Nanocrystal technology, Pharmafreeze, AdvaTab Technology, cotton-candy technology and the like.

In embodiments, the oromucosal dosage forms (e.g., sublingual or buccal or gingival tablet) of the present disclosure may be prepared by sublimation, nanonization, spray drying, granulation including wet granulation, dry granulation or direct compression and the like. U.S. Pat. Nos. 5,178,878, 6,269,615 and 6,221,392 disclose manufacturing friable orally disintegrating tablets by direct compression and packaging in specially designed dome-shaped blister package using a robot-controlled integrated tableting-packaging system; which are herein incorporated by reference in their entirety, for all purposes.

In embodiments, the oromucosal tablet dosage forms as used herein may be prepared by direct compression comprising mixing the active agents (latrepirdine and/or dexmedetomidine) with one or more pharmaceutically acceptable excipients, lubricating the blend and directly compressing into a tablet.

In embodiments, there is provided a process of preparing oromucosal tablet dosage form by dry granulation comprising the steps of:
(i) preparing a mixture containing active agent (latrepirdine and/or dexmedetomidine) and one or more pharmaceutically acceptable excipients;
(ii) compacting the mixture obtained in step (i) to form a granulate,
(iii) optionally mixing the granulate obtained in step (ii) with remaining excipients, and
(iv) subjecting the granulate to compression to obtain the tablet.

In embodiments, the compaction in step (ii) is carried out by roller compaction or slugging techniques.

In embodiments, there is provided a process of preparing oromucosal tablet dosage form by wet granulation comprising the steps of:
(i) preparing a mixture containing active agent (latrepirdine and/or dexmedetomidine) and one or more pharmaceutically acceptable excipients;
(ii) granulating the mixture obtained in step (i) with a suitable granulation liquid to form a wet granulate,
(iii) drying the wet granulate obtained in step (ii),
(iv) optionally mixing the dried granulate obtained in step (iii) with one or more excipients, and
(v) subjecting the granulate obtained in step (iii) or the mixture obtained in step
(iv) to compression to obtain the tablet.

In embodiments, the mixture of step (i) is granulated with any suitable solvent including but not limited to water, an alcohol such as ethanol or isopropyl alcohol, or mixtures thereof.

The dosage form of the present disclosure may be administered to mammals, including humans, as well as non-mammals (e.g., rats, cats and dogs) in need thereof.

In embodiments, latrepirdine or a pharmaceutically acceptable salt thereof and dexmedetomidine or a pharmaceutically acceptable salt thereof are formulated as a sublingual tablet or buccal tablet.

In embodiments, the dosage form comprises a mucoadhesive agent to make the active agent or agents adhere to the oral mucosa. The mucoadhesive agent may possess properties to swell and expand in contact with water thus making tablet disintegrate when wetted with saliva. In embodiments, the dosage form comprises one or more mucoadhesive agents in an amount of about 0.5% to about 30% w/w. For example, the one or more mucoadhesive agents are present in an amount ranging from about 0.5% w/w to about 30% w/w, about 0.5% w/w to about 25% w/w, about 0.5% w/w to about 20% w/w, about 0.5% w/w to about 10% w/w, about 0.5% w/w to about 5% w/w, about 1% w/w to about 30% w/w, about 1% w/w to about 20% w/w, about 1% w/w to about 10% w/w, about 1% w/w to about 5% w/w, about 1% w/w to about 3% w/w, about 1% w/w to about 2% w/w, about 3% w/w to about 30% w/w, about 3% w/w to about 20% w/w, about 3% w/w to about 10% w/w, about 3% w/w to about 5% w/w, about 5% w/w to about 30% w/w, about 5% w/w to about 20% w/w, about 5% w/w to about 10% w/w, about 10% w/w to about 30% w/w, about 10% w/w to about 20% w/w, about 10% w/w to about 15% w/w, about 15% w/w to about 30% w/w, about 15% w/w to about 20% w/w, about 20% w/w to about 30% w/w, about 20% w/w to about 25% w/w, or about 25% w/w to about 30% w/w. In embodiments, the mucoadhesive agent is present in an amount of about 1% w/w to about 5% w/w. In embodiments, the mucoadhesive agent is present in an amount of about 0.5% w/w, about 0.6% w/w, about 0.7% w/w, about 0.8% w/w, about 0.9% w/w, about 1% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w, about 19% w/w, about 20% w/w, about 21% w/w, about 22% w/w, about 23% w/w, about 24% w/w, about 25% w/w, about 26% w/w, about 27% w/w, about 28% w/w, about 29% w/w, or about 30% w/w, including all ranges and values in between.

In embodiments, the mucoadhesive dosage forms have a mucoadhesive strength of at least about 50 dynes/cm², e.g. about 50 dynes/cm², about 75 dynes/cm², about 100 dynes/cm², about 150 dynes/cm², about 200 dynes/cm², about 250 dynes/cm², about 300 dynes/cm², about 350 dynes/cm², about 400 dynes/cm², about 450 dynes/cm², about 500 dynes/cm², about 550 dynes/cm², about 600 dynes/cm², about 650 dynes/cm², about 700 dynes/cm², about 750 dynes/cm², about 800 dynes/cm², about 850 dynes/cm², about 900 dynes/cm², about 950 dynes/cm², or about 1000 dynes/cm², including all ranges and values in between. In embodiments, the mucoadhesive dosage forms have a mucoadhesive strength greater than about 1000 dynes/cm². In embodiments, the dosage form has a mucoadhesive peak force greater than about 50 g, about 100 g, about 200 g, about 300 g, about 400 g, about 500 g, about 600 g, about 700 g, about 800 g, about 900 g, about 1000 g, about 1100 g, about 1200 g, about 1300 g, about 1400 g or about 1500 g. In embodiments, the dosage form has a mucoadhesive peak force of about 50 g, about 100 g, about 200 g, about 300 g, about 400 g, about 500 g, about 600 g, about 700 g, about 800 g, about 900 g, about 1000 g, about 1100 g, about 1200 g, about 1300 g, about 1400 g or about 1500 g, including all ranges and values in between.

In embodiments, suitable mucoadhesive agents as used in the present disclosure include but are not limited to polyacrylic acid polymers (such as carbomers (e.g. with low viscosity), polycarbophil etc), methacrylic acid polymers, cellulose derivatives such as hydroxyethyl cellulose, (HEC), hydroxypropyl cellulose (HPC—such as lower viscosity grades of MVV <150K daltons), ethyl hydroxyethyl cellulose, hydroxypropyl methylcellulose (HPMC-such as grades with lower viscosity like K100L or 4000 cps or less), methyl cellulose, sodium carboxymethyl cellulose, thiolated carboxymethyl cellulose; polysaccharides (such as chitosan, pectin etc); xanthan gum, karaya gum, tragacanth gum; propylene glycol, propylene glycol alginate, sodium alginate, polyethylene oxide (PEO), microcrystalline cellulose (Avicel), croscarmellose, poloxamers (i.e. nonionic triblock copolymers composed of a central hydrophobic chain of polyoxypropylene flanked by two hydrophilic chains of polyoxyethylene; e.g. Poloxamer 407) and mixtures thereof.

In embodiments, the excipients or carriers for inclusion in oromucosal dosage forms are selected from the group consisting of disintegrants, fillers/diluents (matrix forming agents), binders, glidants, lubricants, plasticizers, pH regulators, coloring agents, flavoring agents, taste masking agents, viscosity enhancers, sweetening agents and combinations thereof. Carriers which readily dissolve in saliva are preferred.

In embodiments, examples of suitable disintegrants as used in the present disclosure include but are not limited to cross-linked polyvinyl pyrrolidone, low-substituted hydroxypropyl cellulose, carboxymethyl starch, natural starch, carboxymethyl starch, sodium starch glycolate, pregelatinized starch, dextrins, and other modified starches (starches whose hydroxyl groups have been esterified, hydroxypropyl di-starch phosphate, an enzymatically modified starch, a pregelatinized di-starch phosphate, hydroxyethyl starch, hydroxypropyl starch, a pregelatinized acetylated di-starch phosphate and a pregelatinized purified starch); carboxymethylcellulose calcium, carboxymethylcellulose sodium (or croscarmellose sodium), microcrystalline cellulose, cellulose gum and mixtures thereof. In embodiments, the amount of disintegrant present in the dosage form may range from about 1% w/w to about 5% w/w. For example, the amount of disintegrant present in the dosage form may range from about 1% w/w to about 5% w/w, about 1% w/w to about 4% w/w, about 1% w/w to about 3% w/w, about 1% w/w to about 2% w/w, about 2% w/w to about 5% w/w, about 2% w/w to about 4% w/w, about 2% w/w to about 3% w/w, about 3% w/w to about 5% w/w, about 3% w/w to about 4% w/w or about 4% w/w to about 5% w/w. In embodiments, the disintegrant is present in an amount of about 1% w/w, about 2% w/w, about 3% w/w, about 4% w/w, or about 5% w/w. In embodiments, the disintegrant is present in an amount of about 1% w/w. In embodiments, the disintegrant is present in an amount of about 2% w/w. In embodiments, the disintegrant is present in an amount of about 3% w/w. In embodiments, the disintegrant is present in an amount of about 4% w/w. In embodiments, the disintegrant is present in an amount of about 5% w/w.

In embodiments, examples of suitable diluents/fillers (also called as matrix forming agents) include but are not limited to materials derived from animal or vegetable proteins, such as mammalian gelatin, non-mammalian gelatins, fish gelatin (e.g. high molecular weight gelatin in which more than 50%, more than 60%, or more than 70% of the molecular weight distribution of the gelatin is greater than 30,000 daltons); a standard molecular weight gelatin in which more than substantially 50%, preferably more than 60% and most preferably more than 70% of the molecular weight distribution of the gelatin is below than 30,000 daltons and combinations may be formed wherein the ratio of high molecular weight gelatin to standard molecular weight gelatin (HMW:SMW) ranges substantially from 1:1 to 1:9), dextrins and soy, wheat and psyllium seed proteins; gums such as acacia, guar, agar, and xanthan; polysaccharides; alginates; carboxymethylcelluloses; carrageenans; dextrans; pectins; synthetic polymers such as polyvinylpyrrolidone; and polypeptide/protein or polysaccharide complexes such as gelatin-acacia complexes, starch, mannitol, dicalcium phosphate, potassium sulfate, microcrystalline cellulose, dextrose, lactose, galactose and trehalose; cyclic sugars such as cyclodextrin; inorganic salts such as sodium phosphate, sodium chloride and aluminum silicates; and amino acids having from 2 to 12 carbon atoms such as a glycine, L-alanine, L-aspartic acid, L-glutamic acid, L-hydroxyproline, L-isoleucine, L-leucine and L-phenylalanine and mixtures thereof. In embodiments, the diluents/fillers (or matrix forming agent) are present in a range from about 1% to about 50% w/w of the dosage form. For example, the amount of diluents/fillers present in the dosage form may range from about 1% w/w to about 50% w/w, about 1% w/w to about 20% w/w, about 1% w/w to about 10% w/w, about 1% w/w to about 5% w/w, about 5% w/w to about 50% w/w, about 5% w/w to about 25% w/w, about 5% w/w to about 15% w/w, about 5% w/w to about 10% w/w, about 10% w/w to about 50% w/w, about 10% w/w to about 40% w/w, about 10% w/w to about 30% w/w, about 10% w/w to about 20% w/w, about 10% w/w to about 15% w/w, about 20% w/w to about 50% w/w, about 20% w/w to about 40% w/w, about 20% w/w to about 30% w/w, about 20% w/w to about 25% w/w, about 30% w/w to about 50% w/w, about 30% w/w to about 40% w/w, about 30% w/w to about 35% w/w, about 40% w/w to about 50% w/w, or about 40% w/w to about 45% w/w. In embodiments, the diluents/fillers are present in an amount of about 1% w/w, about 2% w/w, about 3% w/w, about 4% w/w, about 5% w/w, about 6% w/w, about 7% w/w, about 8% w/w, about 9% w/w, about 10% w/w, about 15% w/w, about 20% w/w, about 25% w/w, about 30% w/w, about 35% w/w, about 40% w/w, about 45% w/w, or about 50% w/w, including all ranges and values in between.

In embodiments, examples of suitable binders include but are not limited to starch, pregelatinized starch, PVP (polyvinylpyrrolidone), polyethylene oxide, polyethylene glycol, acacia, alginic acid, tragacanth, sucrose, guar gum, bentonite, cellulose derivatives, such as hydroxypropyl methyl cellulose (HPMC), hydroxypropyl cellulose (HPC) and carboxymethyl cellulose (CMC) and their salts; and mixtures thereof. In embodiments, the binder is present in a range from about 0% to about 20% w/w of the dosage form. For example, the amount of binder present in the dosage form may range from about 1% w/w to about 20% w/w, about 1% w/w to about 10% w/w, about 1% w/w to about 5% w/w, about 5% w/w to about 20% w/w, about 5% w/w to about 15% w/w, about 5% w/w to about 10% w/w, about 10% w/w to about 20% w/w, or about 10% w/w to about 15% w/w. In embodiments, the binder is present in an amount of about 1% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w, about 19% w/w, or about 20% w/w, including all ranges and values in between.

In embodiments, examples of suitable glidants are selected from group comprising calcium phosphate, calcium silicate, powdered cellulose, magnesium silicate, magnesium trisilicate, talc, colloidal silicon dioxide, silica gel, precipitated silica and mixtures thereof. In embodiments, the glidant is present in a range from about 0% to about 5% w/w of the dosage form. For example, the amount of glidant present in the dosage form may range from about 0.1% w/w to about 5% w/w, about 0.1% w/w to about 3% w/w, about 0.1% w/w to about 1% w/w, about 0.5% w/w to about 5% w/w, about 0.5% w/w to about 3% w/w, about 0.5% w/w to about 1% w/w, about 1% w/w to about 5% w/w, about 1% w/w to about 4% w/w, about 1% w/w to about 3% w/w, about 1% w/w to about 2% w/w, about 2% w/w to about 5% w/w, about 2% w/w to about 4% w/w, about 2% w/w to about 3% w/w, about 3% w/w to about 5% w/w, about 3% w/w to about 4% w/w or about 4% w/w to about 5% w/w. In embodiments, the glidant is present in an amount of about 0% w/w, about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1% w/w, about 2% w/w, about 3% w/w, about 4% w/w, or about 5% w/w, including all ranges and values in between.

In embodiments, examples of suitable lubricants include but are not limited to magnesium stearate, calcium stearate, stearic acid, talc, sodium fumarate stearate, sucrose fatty acid esters, aluminum stearate, potassium sodium tartrate, light silicic anhydride, carnauba wax, carmellose calcium, carmellose sodium, hydrated silicon dioxide, hydrogenated oil, hydrogenated rapeseed oil, and mixtures thereof. In embodiments, the lubricant is present in a range from about 0% to about 3% w/w of the dosage form. For example, the amount of lubricant present in the dosage form may range from about 0.1% w/w to about 3% w/w, about 0.1% w/w to about 2% w/w, about 0.1% w/w to about 1% w/w, about 0.5% w/w to about 3% w/w, about 0.5% w/w to about 2% w/w, about 0.5% w/w to about 1% w/w, about 1% w/w to about 3% w/w, about 1% w/w to about 2% w/w, or about 2% w/w to about 3% w/w. In embodiments, the lubricant is present in an amount of about 0% w/w, about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1% w/w, about 2% w/w, or about 3% w/w, including all ranges and values in between.

In embodiments, examples of suitable plasticizers include but are not limited to macrogol, triethyl citrate, acetylated monoglyceride, glycerin, monoacetin, diacetin triacetin, phthalate derivatives like dimethyl, diethyl and dibutyl phthalate polysorbate 80, and propylene glycol, 1,2,3-propanetiol triacetate, hydrogenated starch hydrolysates, corn syrups, distilled acetylated monoglycerides, castor oil derivatives thereof sucrose acetate isobutyrate, and mixtures thereof. In embodiments, the plasticizer is present in a range from about 0% to about 10% w/w of the dosage form. For example, the amount of glidant present in the dosage form may range from about 0.1% w/w to about 10% w/w, about 0.1% w/w to about 5% w/w, about 0.1% w/w to about 3% w/w, about 0.1% w/w to about 1% w/w, about 0.5% w/w to about 10% w/w, about 0.5% w/w to about 5% w/w, about 0.5% w/w to about 3% w/w, about 0.5% w/w to about 1% w/w, about 1% w/w to about 10% w/w, about 1% w/w to about 5% w/w, about 1% w/w to about 4% w/w, about 1% w/w to about 3% w/w, about 1% w/w to about 2% w/w, about 2% w/w to about 10% w/w, about 2% w/w to about 5% w/w, about 2% w/w to about 4% w/w, about 2% w/w to about 3% w/w, about 3% w/w to about 10% w/w, about 3% w/w to about 5% w/w, about 3% w/w to about 4% w/w, about 4% w/w to about 10% w/w, about 4% w/w to about 5% w/w, or about 5% w/w to about 10% w/w. In embodiments, the plasticizer is present in an amount of about 0% w/w, about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1% w/w, about 2% w/w, about 3% w/w, about 4% w/w, about 5% w/w, about 6% w/w, about 7% w/w, about 8% w/w, about 9% w/w, or about 10% w/w, including all ranges and values in between.

In embodiments, examples of suitable pH regulators include but are not limited to inorganic acid (e.g., hydrochloric acid, sulfuric acid, phosphoric acid), an inorganic base (e.g., sodium hydroxide, potassium hydroxide, calcium hydroxide), an organic acid (e.g., citric acid, acetic acid, tartaric acid, succinic acid, boric acid, edetic acid, glucuronic acid, glutaric acid, malic acid, formic acid, gluconic acid, ascorbic acid or fatty acids), and/or an organic base (e.g., ethanolamine, triethanolamine) or mixtures thereof. In embodiments, the pH regulator is present in a range from about 0% to about 2% w/w of the dosage form. For example, the amount of pH regulator present in the dosage form may range from about 0.1% w/w to about 2% w/w, about 0.1% w/w to about 1% w/w, about 0.5% w/w to about 2% w/w, about 0.5% w/w to about 1% w/w, or about 1% w/w to about 2% w/w. In embodiments, pH regulator is present in an amount of about 0% w/w, about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1% w/w, or about 2% w/w, including all ranges and values in between.

In embodiments, examples of suitable coloring agents include but are not limited to food, drug, and cosmetic (FD&C) dyes (FD&C blue, FD&C green, FD&C red, FD&C yellow, FD&C lake), ponceau, indigo drug & cosmetic (D&C) blue, indigo Carmine; iron oxides (e.g. red iron oxide, yellow, black), quinoline yellow, flame red, brilliant red (carmine), carmoisine, sunset yellow and mixtures thereof. In embodiments, the amount of coloring used ranges from about 0% to about 3% w/w of the dosage form. For example, the amount of coloring agent present in the dosage form may range from about 0.1% w/w to about 3% w/w, about 0.1% w/w to about 2% w/w, about 0.1% w/w to about 1% w/w, about 0.5% w/w to about 3% w/w, about 0.5% w/w to about 2% w/w, about 0.5% w/w to about 1% w/w, about 1% w/w to about 3% w/w, about 1% w/w to about 2% w/w, or about 2% w/w to about 3% w/w. In embodiments, the coloring agent is present in an amount of about 0% w/w, about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1% w/w, about 2% w/w, or about 3% w/w, including all ranges and values in between.

In embodiments, examples of suitable flavoring agents include but are not limited to strawberry, apple, pear, peach, plum, orange, pineapple, apricot, lemon, peppermint, black currant, banana, raspberry, raspberry aroma, wild berries, caramel, mint, licorice, grapefruit, caramel, vanilla, cherry and grape flavor, flavoring oils such as cinnamon oil, wintergreen oil, peppermint oil, clove oil, bay oil, anise oil, eucalyptus oil, thyme oil, cedar leave oil, nutmeg oil, sage oil, bitter almond oil and cassia oil and mixtures thereof. In embodiments, the amount of flavoring agent used ranges from about 0% to about 3% w/w of the dosage form. For example, the amount of flavoring agent present in the dosage form may range from about 0.1% w/w to about 3% w/w, about 0.1% w/w to about 2% w/w, about 0.1% w/w to about 1% w/w, about 0.5% w/w to about 3% w/w, about 0.5% w/w to about 2% w/w, about 0.5% w/w to about 1% w/w, about 1% w/w to about 3% w/w, about 1% w/w to about 2% w/w, or about 2% w/w to about 3% w/w. In embodiments, the flavoring agent is present in an amount of about 0% w/w, about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1% w/w, about 2% w/w, or about 3% w/w, including all ranges and values in between.

In embodiments, suitable taste-masking agents include sodium bicarbonate, ion-exchange resins, cyclodextrin inclusion compounds, adsorbates or microencapsulated actives. In embodiments, the amount of taste-masking agent used ranges from about 0% to about 10% w/w of the dosage form. For example, the amount of taste-masking agent present in the dosage form may range from about 0.1% w/w to about 10% w/w, about 0.1% w/w to about 5% w/w, about 0.1% w/w to about 3% w/w, about 0.1% w/w to about 1% w/w, about 0.5% w/w to about 10% w/w, about 0.5% w/w to about 5% w/w, about 0.5% w/w to about 3% w/w, about 0.5% w/w to about 1% w/w, about 1% w/w to about 10% w/w, about 1% w/w to about 5% w/w, about 1% w/w to about 4% w/w, about 1% w/w to about 3% w/w, about 1% w/w to about 2% w/w, about 2% w/w to about 10% w/w, about 2% w/w to about 5% w/w, about 2% w/w to about 4% w/w, about 2% w/w to about 3% w/w, about 3% w/w to about 10% w/w, about 3% w/w to about 5% w/w, about 3% w/w to about 4% w/w, about 4% w/w to about 10% w/w, about 4% w/w to about 5% w/w, or about 5% w/w to about 10% w/w. In embodiments, the taste-masking agent is present in an amount of about 0% w/w, about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1% w/w, about 2% w/w, about 3% w/w, about 4% w/w, about 5% w/w, about 6% w/w, about 7% w/w, about 8% w/w, about 9% w/w, or about 10% w/w, including all ranges and values in between.

In embodiments, suitable viscosity enhancers include but are not limited to polymers, sugars, sugar alcohols, gums, clays, silicas, and the like. In embodiments, the amount of viscosity enhancers used ranges from about 0% to about 65% w/w of the dosage form. For example, the amount of viscosity enhancers present in the dosage form may range from about 0.1% w/w to about 65% w/w, about 0.1% w/w to about 50% w/w, about 0.1% w/w to about 20% w/w, about 0.1% w/w to about 10% w/w, about 0.1% w/w to about 5% w/w, about 5% w/w to about 65% w/w, about 5% w/w to about 50% w/w, about 5% w/w to about 25% w/w, about 5% w/w to about 15% w/w, about 5% w/w to about 10% w/w, about 10% w/w to about 65% w/w, about 10% w/w to about 50% w/w, about 10% w/w to about 40% w/w, about 10% w/w to about 30% w/w, about 10% w/w to about 20% w/w, about 10% w/w to about 15% w/w, about 20% w/w to about 65% w/w, about 20% w/w to about 50% w/w, about 20% w/w to about 40% w/w, about 20% w/w to about 30% w/w, about 20% w/w to about 25% w/w, about 30% w/w to about 65% w/w, about 30% w/w to about 50% w/w, about 30% w/w to about 40% w/w, about 30% w/w to about 35% w/w, about 40% w/w to about 65% w/w, about 40% w/w to about 50% w/w, about 40% w/w to about 45% w/w, about 50% w/w to about 65% w/w, about 50% w/w to about 60% w/w, or about 50% w/w to about 55% w/w. In embodiments, the viscosity enhancer is present in an amount of about 0% w/w, about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1% w/w, about 2% w/w, about 3% w/w, about 4% w/w, about 5% w/w, about 6% w/w, about 7% w/w, about 8% w/w, about 9% w/w, about 10% w/w, about 15% w/w, about 20% w/w, about 25% w/w, about 30% w/w, about 35% w/w, about 40% w/w, about 45% w/w, about 50% w/w, about 55% w/w, about 60% w/w, or about 65% w/w, including all ranges and values in between.

In embodiments, examples of suitable sweetening agents include but are not limited to fructose, sucrose, glucose, maltose, sorbitol, erythritol, xylitol, aspartame, stevia extract, glycyrrhiza, mogroside, sodium cyclamate, saccharine, saccharine sodium, acesulfame, dextrose, sucralose, monosodium glycyrrhizinate, monoamonium glycyrrhizinate, isomalt, glycerine, dipotassium glycyrrhizinate, thaumatin and mixtures thereof. In embodiments, the amount of sweetening agent ranges from about 0.5 to about 2% w/w of the dosage form. For example, the amount of sweetening agents present in the dosage form may range from about 0.5% w/w to about 2% w/w, about 0.5% w/w to about 1% w/w, or about 1% w/w to about 2% w/w. In embodiments, the sweetening agents are present in an amount of about 0.5% w/w, about 0.6% w/w, about 0.7% w/w, about 0.8% w/w, about 0.9% w/w, about 1% w/w, or about 2% w/w, including all ranges and values in between.

In embodiments, the present disclosure provides an oromucosal dosage form comprising:
(i) therapeutically effective amount of latrepirdine or a pharmaceutically acceptable salt thereof;
(ii) one or more mucoadhesive agents and
(iii) one or more pharmaceutically acceptable excipients or carriers;
wherein the dosage form disintegrates in not less than about 1 minute upon contact with an oral mucosa. In embodiments, the dosage form is lyophilized (freeze-dried).

In embodiments, the mucoadhesive is sodium alginate. In embodiments, the mucoadhesive is a carbomer.

In embodiments, there is provided an oromucosal (e.g., sublingual or buccal or gingival) lyophilized tablet comprising: (i) therapeutically effective amount of latrepirdine or a pharmaceutically acceptable salt thereof; (ii) sodium alginate; (iii) croscarmellose sodium or sodium starch glycolate; (iv) sucralose; (v) magnesium stearate; (vi) lactose monohydrate and (vii) optionally other pharmaceutical acceptable excipients. In embodiments, the tablet disintegrates in more than about 1 minute upon contact with an oral mucosa.

In embodiments, there is provided an oromucosal (e.g., sublingual or buccal or gingival) lyophilized tablet comprising: (i) therapeutically effective amount of latrepirdine or a pharmaceutically acceptable salt thereof; (ii) sodium alginate (iii) croscarmellose sodium or sodium starch glycolate; (iv) sucralose; (v) silicon dioxide; (vi) mannitol and (vii) optionally other pharmaceutical acceptable excipients. In embodiments, the tablet disintegrates in more than about 1 minute upon contact with an oral mucosa.

In embodiments, there is provided an oromucosal (e.g., sublingual or buccal or gingival) lyophilized tablet comprising: (i) therapeutically effective amount of latrepirdine or a pharmaceutically acceptable salt thereof; (ii) carbomer; (iii) croscarmellose sodium or sodium starch glycolate; (iv) sucralose; (v) magnesium stearate; (vi) mannitol and (vii) optionally other pharmaceutical acceptable excipients. In embodiments, the tablet disintegrates in not less than about 1 minute upon contact with an oral mucosa.

In embodiments, there is provided an oromucosal (e.g., sublingual or buccal) lyophilized tablet comprising: (i) therapeutically effective amount of latrepirdine or a pharmaceutically acceptable salt thereof; (ii) carbomer; (iii) croscarmellose sodium or sodium starch glycolate; (iv) sucralose; (v) silicon dioxide; (vi) lactose monohydrate and (vii) optionally other pharmaceutical acceptable excipients. In embodiments, the tablet disintegrates in not less than about 1 minute upon contact with an oral mucosa.

In embodiments, the mucoadhesive is xanthan gum.

In embodiments, there is provided an oromucosal (e.g., sublingual or buccal) lyophilized tablet comprising: (i) therapeutically effective amount of latrepirdine or a pharmaceutically acceptable salt thereof; (ii) xanthan gum; (iii) croscarmellose sodium or sodium starch glycolate; (iv) sucralose; (v) magnesium stearate; (vi) lactose monohydrate and (vii) optionally other pharmaceutical acceptable excipients. In embodiments, the tablet disintegrates in not less than about 1 minute upon contact with an oral mucosa.

In embodiments, there is provided an oromucosal (e.g., sublingual or buccal) lyophilized tablet comprising: (i) therapeutically effective amount of latrepirdine or a pharmaceutically acceptable salt thereof; (ii) xanthan gum; (iii) croscarmellose sodium or sodium starch glycolate; (iv) sucralose; (v) silicon dioxide; (vi) mannitol and (vii) optionally other pharmaceutical acceptable excipients. In embodiments, the tablet disintegrates in not less than about 1 minute upon contact with an oral mucosa.

In embodiments, the dosage form is a sublingual tablet and is elliptical or oval. In embodiments, the dosage form is a buccal tablet and is oval in shape. In embodiments, the dosage form is used for the treatment of agitation. In embodiments, the dosage form is used in reducing noradrenergic hyperarousal. In embodiments, the dosage form is used for the treatment of depression.

In embodiments, the present disclosure provides an oromucosal tablet dosage form comprising:
(i) a therapeutically effective amount of dexmedetomidine or a pharmaceutically acceptable salt thereof;
(ii) one or more mucoadhesive agents and
(iii) one or more pharmaceutically acceptable excipients or carriers.

In embodiments, the tablet disintegrates in not less than about 1 minute upon contact with an oral mucosa.

In embodiments, the present disclosure provides an oromucosal tablet dosage form comprising:
(i) a therapeutically effective amount of latrepirdine or a pharmaceutically acceptable salt thereof;
(ii) one or more mucoadhesive agents and
(iii) one or more pharmaceutically acceptable excipients or carriers.

In embodiments, the tablet disintegrates in not less than about 1 minute upon contact with an oral mucosa.

In embodiments, the present disclosure provides an oromucosal dosage form comprising:
(i) a therapeutically effective amount of latrepirdine or a pharmaceutically acceptable salt thereof;
(ii) a therapeutically effective amount of dexmedetomidine or a pharmaceutically acceptable salt thereof;
(iii) one or more mucoadhesive agents and
(iv) one or more pharmaceutically acceptable excipients or carriers.

In embodiments, the tablet disintegrates within about 5 seconds to about 10 minutes upon contact with an oral mucosa.

In embodiments, the dosage form is lyophilized (freeze dried).

In embodiments, the dosage form is used for the treatment of agitation caused by noradrenergic hyperarousal. In embodiments, the agitation is treated in a subject without causing significant sedation. In embodiments, the dosage form is used for the treatment of depression in a subject.

In embodiments, the present disclosure provides an oromucosal (sublingual or buccal) lyophilized tablet comprising (i) a therapeutically effective amount of latrepirdine or a pharmaceutically acceptable salt thereof; (ii) a therapeutically effective amount of dexmedetomidine or a pharmaceutically acceptable salt thereof; (iii) sodium alginate, xanthan gum, carbomer, hydroxypropyl cellulose, hydroxypropyl methylcellulose, or polyethylene oxide; (iv) croscarmellose sodium or sodium starch glycolate; (v) sucralose; (vi) magnesium stearate and/or silicon dioxide; (vii) lactose or mannitol; and (viii) optionally other pharmaceutical acceptable excipients. In embodiments, the tablet disintegrates in not less than about 1 minute upon contact with an oral mucosa.

In embodiments, the oromucosal tablet comprises about 5 micrograms to about 300 micrograms of dexmedetomidine or a pharmaceutically acceptable salt thereof and about 0.1 mg to about 100 mg of latrepirdine per unit. In embodiments, the oromucosal tablet comprises about 10 micrograms to 240 micrograms of dexmedetomidine or a pharmaceutically acceptable salt thereof (e.g. about 30 micrograms, about 60 micrograms, about 90 micrograms, about 120 micrograms, 180 micrograms, about 210 micrograms or about 240 micrograms, including all ranges and values in between) and about 1 mg to about 50 mg of latrepirdine or a pharmaceutically acceptable salt thereof per unit (e.g. about 5 mg, about 10 mg, about 15 mg, about 20 mg, about 25 mg, about 30 mg, about 35 mg, about 40 mg, about 45 mg or about 50 mg, including all ranges and values in between).

In embodiments, the tablet is administered sublingually, buccally or gingivally. In embodiments, the dosage form is administered via a single dosage form or via multiple dosage forms.

IV. Methods and Administration

In embodiments, the present disclosure provides methods of treating agitation in an agitated subject, comprising administering a therapeutically effective amount of latrepirdine or a pharmaceutically acceptable salt thereof to the subject. In embodiments, the therapeutically effective amount of latrepirdine or a pharmaceutically acceptable salt thereof is present in a dosage form as described herein.

In embodiments, the present disclosure provides a method of treating agitation in an agitated subject, comprising oromucosally administering a therapeutic effective amount of latrepirdine or a pharmaceutically acceptable salt thereof to the subject. In embodiments, latrepirdine or a pharmaceutically acceptable salt thereof is administered (e.g. daily) for at least one week, at least two weeks, at least three weeks, at least four weeks, at least one month, at least two months, at least three months, at least four months, at least five months, at least six months or at least one year.

In embodiments, the present disclosure provides a method of treating agitation in an agitated subject, comprising administering a therapeutic effective amount of latrepirdine or a pharmaceutically acceptable salt thereof in combination with a therapeutic effective amount of dexmedetomidine or a pharmaceutically acceptable salt (e.g. dexmedetomidine hydrochloride) thereof to the subject. In embodiments, the treatment is effective with reduced or no side effects (e. g. cardiac or respiratory side effects). In embodiments, the therapeutically effective amount of latrepirdine or a pharmaceutically acceptable salt thereof is present in a dosage form as described herein. In embodiments, the therapeutically effective amount of dexmedetomidine or a pharmaceutically acceptable salt thereof is present in a dosage form as described herein. In embodiments, the therapeutically effective amount of latrepirdine and dexmedetomidine or a pharmaceutically acceptable salt thereof are present in the same dosage form or in separate dosage forms as described herein.

In embodiments, the active agents dexmedetomidine and latrepirdine are administered concurrently (same dosage form or separate dosage form) to the subject for a specific period of time (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days or so on) followed by single agent administration of latrepirdine to the subject for a specific period of time (e.g., 1, 2, 3, 4, 5, 6, 7, 8, 9, 10, 11, 12 months or so on).

The present disclosure provides a method of treating agitation in an agitated subject, comprising oromucosally administering a therapeutic effective amount of latrepirdine or a pharmaceutically acceptable salt thereof in combination with a therapeutic effective amount of dexmedetomidine or a pharmaceutically acceptable salt (e.g. hydrochloride salt) thereof to the subject. In embodiments, the treatment is effective with reduced or no side effects (e. g. cardiac or respiratory side effects). In embodiments, the single administration of the combination treats agitation and maintains calming effect for at least 12 hours. In embodiments, the therapeutically effective amount of latrepirdine or a pharmaceutically acceptable salt thereof is present in a dosage form as described herein. In embodiments, the therapeutically effective amount of dexmedetomidine or a pharmaceutically acceptable salt thereof is present in a dosage form as described herein. In embodiments, the therapeutically effective amount of latrepirdine or a pharmaceutically acceptable salt thereof and dexmedetomidine or a pharmaceutically acceptable salt thereof are present in the same dosage form or in separate dosage forms as described herein.

In embodiments, the agitation can be acute agitation, chronic agitation or both.

In embodiments, the agitation is caused by noradrenergic hyperarousal.

In embodiments, the agitation is treated without causing any significant sedation.

In embodiments, the agitation is associated with neuropsychiatric disorders selected from schizophrenia, bipolar disorder, bipolar mania, delirium, depression or other related neuropsychiatric disorders.

In embodiments, the agitation is associated with neurodegenerative disorders selected from Alzheimer's disease, frontotemporal dementia (or Pick's disease), dementia, dementia with Lewy bodies, post-traumatic stress disorder, Parkinson's disease, vascular dementia, vascular cognitive impairment, Huntington's disease, multiple sclerosis, Creutzfeldt-Jakob disease, multiple system atrophy, progressive supranuclear palsy or other related neurodegenerative disorders.

In embodiments, the agitation is associated with alcohol withdrawal or substance abuse withdrawal including opioid withdrawal.

The present disclosure provides a method of treating chronic agitation in a subject, comprising oromucosally administering a therapeutic effective amount of latrepirdine or a pharmaceutically acceptable salt thereof in combination with a therapeutic effective amount of dexmedetomidine or a pharmaceutically acceptable salt (e.g. dexmedetomidine hydrochloride) thereof to the subject. In embodiments, the subject is suffering from dementia. In embodiments, dementia include Alzheimer's disease dementia (AD), Fronto-temporal dementia (FTD), Vascular dementia, Lewy body disease (LBD), and Down dementia.

In embodiments, the disclosure provides a method of treating chronic agitation in a subject, comprising oromucosally administering to the subject a dosage form comprising a therapeutically effective amount of latrepirdine or a pharmaceutically acceptable salt thereof. In embodiments, the disclosure provides a method of treating chronic agitation in a subject, comprising oromucosally administering to the subject a dosage form comprising a therapeutically effective amount of latrepirdine or a pharmaceutically acceptable salt thereof in combination with therapeutic effective amount of dexmedetomidine or a pharmaceutically acceptable salt. In embodiments, the subject is suffering from dementia. In embodiments, dementia includes Alzheimer's disease dementia (AD), Fronto-temporal dementia (FTD), Vascular dementia, Lewy body disease (LBD), and Down dementia.

In embodiments, the disclosure provides a method of treating agitation in an agitated subject, comprising oromucosally administering to the subject a dosage form comprising a therapeutically effective amount of latrepirdine or a pharmaceutically acceptable salt thereof. In embodiments, the agitation is caused by noradrenergic hyperarousal. In embodiments, the therapeutically effective amount of latrepirdine or a pharmaceutically acceptable salt thereof is present in a dosage form as described herein. In embodiments, the therapeutically effective amount of dexmedetomidine or a pharmaceutically acceptable salt thereof is present in a dosage form as described herein.

In embodiments, the agitation is severe. In embodiments, the agitation is mild.

In embodiments, the disclosure provides a method of treatment of agitation in a subject comprising oromucosally administering a therapeutically effective amount of latrepirdine or a pharmaceutically acceptable salt thereof, wherein the subject has a concomitant Alzheimer's disease.

In embodiments, the disclosure provides a method of treatment of agitation in a subject comprising oromucosally administering a therapeutically effective amount of latrepirdine or a pharmaceutically acceptable salt thereof in combination with a therapeutic effective amount of dexmedetomidine or a pharmaceutically acceptable salt, wherein the subject has a concomitant Alzheimer's disease.

In embodiments, the disclosure provides a method of treatment of agitation in a subject comprising oromucosally administering a therapeutically effective amount of latrepirdine or a pharmaceutically acceptable salt thereof, wherein the subject has a concomitant dementia.

In embodiments, the disclosure provides a method of treatment of agitation in a subject comprising oromucosally administering a therapeutically effective amount of latrepirdine or a pharmaceutically acceptable salt thereof in combination with a therapeutic effective amount of dexmedetomidine or a pharmaceutically acceptable salt, wherein the subject has a concomitant Dementia.

In embodiments, the disclosure provides a method of treatment of agitation in a subject comprising oromucosally administering a therapeutically effective amount of latrepirdine or a pharmaceutically acceptable salt thereof, wherein the subject has a concomitant Parkinson's disease.

In embodiments, the disclosure provides a method of treatment of agitation in a subject comprising oromucosally administering a therapeutically effective amount of latrepirdine or a pharmaceutically acceptable salt thereof in combination with a therapeutic effective amount of dexmedetomidine or a pharmaceutically acceptable salt, wherein the subject has a concomitant Parkinson's disease.

In embodiments, the disclosure provides a method of treatment of agitation in a subject comprising oromucosally administering a therapeutically effective amount of latrepirdine or a pharmaceutically acceptable salt thereof, wherein the subject has a concomitant PTSD.

In embodiments, the disclosure provides a method of treatment of agitation in a subject comprising oromucosally administering a therapeutically effective amount of latrepirdine or a pharmaceutically acceptable salt thereof in combination with a therapeutic effective amount of dexmedetomidine or a pharmaceutically acceptable salt, wherein the subject has a concomitant PTSD.

In embodiments, the disclosure provides a method of treatment of agitation in a subject comprising oromucosally administering a therapeutically effective amount of latrepirdine or a pharmaceutically acceptable salt thereof, wherein the subject has a concomitant vascular cognitive impairment.

In embodiments, the disclosure provides a method of treatment of agitation in a subject comprising oromucosally administering a therapeutically effective amount of latrepirdine or a pharmaceutically acceptable salt thereof in combination with a therapeutic effective amount of dexmedetomidine or a pharmaceutically acceptable salt, wherein the subject has a concomitant vascular cognitive impairment.

In embodiments, the disclosure provides a method of treatment of agitation in a subject comprising oromucosally administering a therapeutically effective amount of latrepirdine or a pharmaceutically acceptable salt thereof, wherein the subject has a concomitant Huntington's disease.

In embodiments, the disclosure provides a method of treatment of agitation in a subject comprising oromucosally administering a therapeutically effective amount of latrepirdine or a pharmaceutically acceptable salt thereof in combination with a therapeutic effective amount of dexmedetomidine or a pharmaceutically acceptable salt, wherein the subject has a concomitant Huntington's disease.

In embodiments, the disclosure provides a method of treatment of agitation in a subject comprising oromucosally administering a therapeutically effective amount of latrepirdine or a pharmaceutically acceptable salt thereof, wherein the subject has a concomitant schizophrenia.

In embodiments, the disclosure provides a method of treatment of agitation in a subject comprising oromucosally administering a therapeutically effective amount of latrepirdine or a pharmaceutically acceptable salt thereof in combination with a therapeutic effective amount of dexmedetomidine or a pharmaceutically acceptable salt, wherein the subject has a concomitant schizophrenia

In embodiments, the disclosure provides a method of treatment of agitation in a subject comprising oromucosally administering a therapeutically effective amount of latrepirdine or a pharmaceutically acceptable salt thereof, wherein the subject has a concomitant bipolar disorder.

In embodiments, the disclosure provides a method of treatment of agitation in a subject comprising oromucosally administering a therapeutically effective amount of latrepirdine or a pharmaceutically acceptable salt thereof in combination with a therapeutic effective amount of dexmedetomidine or a pharmaceutically acceptable salt, wherein the subject has a concomitant bipolar disorder.

In embodiments, the disclosure provides a method of treatment of agitation in a subject comprising oromucosally administering a therapeutically effective amount of latrepirdine or a pharmaceutically acceptable salt thereof, wherein the subject has a concomitant depression.

In embodiments, the disclosure provides a method of treatment of agitation in a subject comprising oromucosally administering a therapeutically effective amount of latrepirdine or a pharmaceutically acceptable salt thereof in combination with a therapeutic effective amount of dexmedetomidine or a pharmaceutically acceptable salt, wherein the subject has a concomitant depression

In embodiments, the disclosure provides a method of treatment of agitation in a subject comprising oromucosally administering a therapeutically effective amount of latrepirdine or a pharmaceutically acceptable salt thereof, wherein the subject has a concomitant delirium.

In embodiments, the disclosure provides a method of treatment of agitation in a subject comprising oromucosally administering a therapeutically effective amount of latrepirdine or a pharmaceutically acceptable salt thereof in combination with a therapeutic effective amount of dexmedetomidine or a pharmaceutically acceptable salt, wherein the subject has a concomitant delirium.

In embodiments, the present disclosure provides a method of treating agitation in an agitated subject, comprising oromucosally administering to the subject:
(i) about 5 micrograms to about 300 micrograms of dexmedetomidine or a pharmaceutically acceptable salt thereof (e.g. dexmedetomidine hydrochloride) and
(ii) about 0.1 mg to about 100 mg of latrepirdine or a pharmaceutically acceptable salt thereof.

In embodiments, the agitation is treated without also inducing significant sedation. In embodiments, the agitation is caused by noradrenergic hyperarousal. In embodiments, the therapeutically effective amount of latrepirdine or a pharmaceutically acceptable salt thereof and dexmedetomidine or a pharmaceutically acceptable salt thereof are present in the same dosage form or in separate dosage forms as described herein.

In embodiments, the present disclosure provides a method of treating agitation in an agitated subject, comprising oromucosally administering to the subject:
(i) about 5 micrograms to about 200 micrograms of dexmedetomidine or a pharmaceutically acceptable salt thereof (e.g. dexmedetomidine hydrochloride) and
(ii) about 5 mg to about 50 mg of latrepirdine or a pharmaceutically acceptable salt thereof.

In embodiments, the agitation is treated without also inducing significant sedation. In embodiments, the agitation is caused by noradrenergic hyperarousal. In embodiments, the therapeutically effective amount of latrepirdine or a pharmaceutically acceptable salt thereof and dexmedetomidine or a pharmaceutically acceptable salt thereof are present in the same dosage form or in separate dosage forms as described herein.

In embodiments, the present disclosure provides a method of reducing noradrenergic hyperarousal comprising oromucosally administering a therapeutically effective amount of latrepirdine or a pharmaceutically acceptable salt thereof to a subject.

In embodiments, the present disclosure provides a method of reducing noradrenergic hyperarousal comprising oromucosally administering a therapeutically effective amount of latrepirdine or a pharmaceutically acceptable salt thereof and a therapeutically effective amount of dexmedetomidine or a pharmaceutically acceptable salt thereof (e.g., dexmedetomidine hydrochloride) to a subject.

In embodiments, dexmedetomidine and latrepirdine are administered together in a single dosage form. In embodiments, dexmedetomidine and latrepirdine are administered conjointly in separate dosage forms. In embodiments, dexmedetomidine and latrepirdine or pharmaceutically acceptable salts thereof are administered as a single dose via single unit dosage form or multiple unit dosage forms administered simultaneously. In embodiments, dexmedetomidine or a salt thereof is administered in one or more-unit doses up to a total daily dose of about 0.5 micrograms to about 500 micrograms. In embodiments, latrepirdine or a salt thereof is administered in one or more-unit doses up to a total daily dose of about 1 mg to about 100 mg.

In embodiments, the present disclosure provides a method of treatment comprising administering dexmedetomidine or a pharmaceutically acceptable salt to a subject in an oromucosal dosage form that provides rapid relief of agitation and then continuing treatment with latrepirdine or a pharmaceutically acceptable salt for an effective period of time.

In embodiments, the present disclosure provides a method of treatment of behavioral and psychological symptoms in subjects with neurodegenerative disorder, comprising oromucosally administering a therapeutically effective amount of latrepirdine or a pharmaceutically acceptable salt thereof. In embodiment, the behavioral and psychological symptom includes agitation or aggression.

In embodiments, the present disclosure provides a method of treatment of behavioral and psychological symptoms in subjects with neurodegenerative disorder, comprising oromucosally administering a therapeutically effective amounts of latrepirdine and dexmedetomidine or pharmaceutically acceptable salts thereof. In embodiment, the behavioral and psychological symptom includes agitation or aggression.

In embodiments, the present disclosure provides a method of treatment of behavioral and psychological symptoms in subjects with a neuropsychiatric disorder, comprising oromucosally administering a therapeutically effective amount of latrepirdine or a pharmaceutically acceptable salt thereof. In embodiment, the behavioral and psychological symptom includes agitation or aggression.

In embodiments, the present disclosure provides a method of treatment of behavioral and psychological symptoms in subjects with a neuropsychiatric disorder, comprising oromucosally administering a therapeutically effective amounts of latrepirdine and dexmedetomidine or pharmaceutically acceptable salts thereof. In embodiment, the behavioral and psychological symptom includes agitation or aggression.

In embodiments, the present disclosure provides use of therapeutically effective amount of latrepirdine or a pharmaceutically acceptable salt thereof for the treatment of behavioral and psychological symptoms in subjects with a neurodegenerative disorder.

In embodiments, the present disclosure provides use of therapeutically effective amounts of latrepirdine and dexmedetomidine or pharmaceutically acceptable salts thereof for the treatment of behavioral and psychological symptoms in subjects with a neurodegenerative disorder.

In embodiments, the present disclosure provides use of therapeutically effective amount of latrepirdine or a pharmaceutically acceptable salt thereof for the treatment of behavioral and psychological symptoms in subjects with a neuropsychiatric disorder.

In embodiments, the present disclosure provides use of therapeutically effective amounts of latrepirdine and dexmedetomidine or pharmaceutically acceptable salts thereof for the treatment of behavioral and psychological symptoms in subjects with a neuropsychiatric disorder.

In embodiments, the present disclosure provides a method of treatment of depression in a subject in need thereof, comprising administering oromucosally to the subject a dosage form comprising a therapeutically effective amount of latrepirdine or a pharmaceutically acceptable salt thereof. In embodiments, latrepirdine is administered in a dosage amount of about 1 mg to about 100 mg once a day. In embodiments, latrepirdine is administered in a dosage amount of about 10 mg once a day. In embodiments, latrepirdine is administered in a dosage amount of about 20 mg once a day. In embodiments, latrepirdine is administered in a dosage amount of about 30 mg once a day. In embodiments, latrepirdine is administered in a dosage amount of about 20 mg twice a day. In embodiments, latrepirdine is administered in a dosage amount of about 20 mg thrice a day. In embodiments, latrepirdine is administered in a dosage amount of about 30 mg once a day. In embodiments, latrepirdine is administered in a dosage amount of about 30 mg twice a day. In embodiments, latrepirdine is administered to the subject in a total daily dose of about 60 mg

The present disclosure provides a method of treating depression in a subject in need thereof, the method comprising administering oromucosally to the subject a therapeutically effective amount of dexmedetomidine or a pharmaceutically acceptable salt thereof.

The present disclosure provides a method of treating depression in a subject in need thereof, the method comprising administering to the subject a therapeutically effective amount of dexmedetomidine or a pharmaceutically acceptable salt thereof in combination with a therapeutically effective amount of latrepirdine or a pharmaceutically acceptable salt thereof. The present disclosure provides a method of treating depression in a subject in need thereof, the method comprising administering oromucosally to the subject a therapeutically effective amount of dexmedetomidine or a pharmaceutically acceptable salt thereof in combination with a therapeutically effective amount of latrepirdine or a pharmaceutically acceptable salt thereof.

In embodiments, the present disclosure provides a method of treating depression in a subject in need thereof, the method comprising administering oromucosally to the subject about 1 mg to about 100 mg of latrepirdine or a pharmaceutically acceptable salt thereof and about 5 micrograms to about 300 micrograms of dexmedetomidine or a pharmaceutically acceptable salt thereof

In embodiments, the present disclosure provides a method of treating depression in a subject in need thereof, the method comprising administering oromucosally to the subject about 10 mg of latrepirdine or a pharmaceutically acceptable salt thereof and about 30 micrograms of dexmedetomidine or a pharmaceutically acceptable salt thereof

In embodiments, the present disclosure provides a method of treating depression in a subject in need thereof, the method comprising administering oromucosally to the subject about 10 mg of latrepirdine or a pharmaceutically acceptable salt thereof and about 40 micrograms of dexmedetomidine or a pharmaceutically acceptable salt thereof

In embodiments, the present disclosure provides a method of treating depression in a subject in need thereof, the method comprising administering oromucosally to the subject about 10 mg of latrepirdine or a pharmaceutically acceptable salt thereof and about 60 micrograms of dexmedetomidine or a pharmaceutically acceptable salt thereof.

In embodiments, the present disclosure provides a method of treating depression in a subject in need thereof, the method comprising administering oromucosally to the subject about 10 mg of latrepirdine or a pharmaceutically acceptable salt thereof and about 80 micrograms of dexmedetomidine or a pharmaceutically acceptable salt thereof.

In embodiments, the present disclosure provides a method of treating depression in a subject in need thereof, the method comprising administering oromucosally to the subject about 20 mg of latrepirdine or a pharmaceutically acceptable salt thereof and about 30 micrograms of dexmedetomidine or a pharmaceutically acceptable salt thereof

In embodiments, the present disclosure provides a method of treating depression in a subject in need thereof, the method comprising administering oromucosally to the subject about 20 mg of latrepirdine or a pharmaceutically acceptable salt thereof and about 40 micrograms of dexmedetomidine or a pharmaceutically acceptable salt thereof.

In embodiments, the present disclosure provides a method of treating depression in a subject in need thereof, the method comprising administering oromucosally to the subject about 20 mg of latrepirdine or a pharmaceutically acceptable salt thereof and about 60 micrograms of dexmedetomidine or a pharmaceutically acceptable salt thereof.

In embodiments, the present disclosure provides a method of treating depression in a subject in need thereof, the method comprising administering oromucosally to the subject about 20 mg of latrepirdine or a pharmaceutically acceptable salt thereof and about 80 micrograms of dexmedetomidine or a pharmaceutically acceptable salt thereof.

In embodiments, the improvement in depressive symptoms is observed as measured by HAM-D-17 depression subscale.

In embodiments, the subject has a HAM-D-17 total score ≥18 at the start of treatment.

In embodiments, there is provided a method of reducing score on HDRS scale in a human subject suffering from depression comprising administering oromucosally effective amounts of dexmedetomidine either alone or in combination with latrepirdine or pharmaceutically acceptable salts thereof.

In embodiments, there is provided a method of reducing score on MADRS scale in a human subject suffering from depression comprising administering oromucosally effective amounts of dexmedetomidine either alone or in combination with latrepirdine or pharmaceutically acceptable salts thereof.

HAM-D or HDRS is used as an instrument for assessing the symptoms of depression. The instrument is administered by clinicians after a structured or unstructured interview of the patient to determine their symptoms. A total score is calculated by summing the individual scores from each question. Scores below 7 generally represent the absence or remission of depression. Scores between 7-17 represent mild depression. Scores between 18-24 represent moderate depression. Scores 25 and above represent severe depression. Most of the studies of depression consider a patient to have experienced ‘response’ to treatment if the score decreases by more than 50%. Remission′ is commonly understood to be a score below 7.

The Montgomery-Åsberg Depression Rating Scale (MADRS) is a ten-item diagnostic questionnaire which psychiatrists use to measure the severity of depressive episodes in patients with mood disorders. Higher MADRS score indicates more severe depression, and each item yields a score of 0 to 6. The overall score ranges from 0 to 60. The questionnaire includes questions on the following symptoms 1. Apparent sadness 2. Reported sadness 3. Inner tension 4. Reduced sleep 5. Reduced appetite 6. Concentration difficulties 7. Lassitude 8. Inability to feel 9. Pessimistic thoughts 10. Suicidal thoughts.

In embodiments, the unit dosage forms comprising dexmedetomidine or a pharmaceutically acceptable salt thereof and latrepirdine are administered simultaneously at the same time or within a short period of time, usually less than 1 hour, preferably 0.5 hours, more preferably 0.25 hours.

In embodiments, the unit dosage forms comprising dexmedetomidine or a pharmaceutically acceptable salt thereof and latrepirdine are administered sequentially separated by a time-period anywhere within about 24 hours, e.g., about 12 hours, about 11 hours, about 10 hours, about 9 hours, about 8 hours, about 7 hours, about 6 hours, about 5 hours, about 4 hours, about 3 hours, about 2 hours or about 1 hour of each other.

In embodiments, the combination comprising latrepirdine and dexmedetomidine or salts thereof is administered once daily, twice daily, thrice daily or four times, five times, six times a day, preferably once, twice or thrice daily.

In embodiments, the combination comprising latrepirdine and dexmedetomidine or salts thereof is administered for at least 3 days, at least 5 days, at least 7 days, at least 10 days, at least 15 days, at least 30 days, at least 60 days, at least 90 days, at least 180 days, at least 365 days, or longer.

In embodiments, the depression is moderate or severe. In embodiments, the depression is major depression, bipolar disorder or mixed depression.

In embodiments, the present disclosure provides a synergistic combination comprising:
(i) a therapeutically effective amount of latrepirdine or a pharmaceutically acceptable salt thereof and
(ii) a therapeutically effective amount of dexmedetomidine or a pharmaceutically acceptable salt thereof, for the treatment of depression in a subject in need thereof.

In embodiments, the present disclosure provides a method of treating psychosis in a subject in need thereof, comprising administering a therapeutically effective amount of latrepirdine or a pharmaceutically acceptable salt thereof to the subject. In embodiments, latrepirdine is administered in a dosage amount of about 1 mg to about 100 mg once a day. In embodiments, latrepirdine is administered in a dosage amount of about 10 mg once a day. In embodiments, latrepirdine is administered in a dosage amount of about 20 mg once a day. In embodiments, latrepirdine is administered in a dosage amount of about 30 mg once a day. In embodiments, latrepirdine is administered in a dosage amount of about 10 mg twice a day. In embodiments, latrepirdine is administered in a dosage amount of about 20 mg twice a day. In embodiments, latrepirdine is administered in a dosage amount of about 30 mg twice a day. In embodiments, latrepirdine is administered in a dosage amount of about 10 mg thrice a day. In embodiments, latrepirdine is administered in a dosage amount of about 20 mg thrice a day. In embodiments, latrepirdine is administered in a dosage amount of about 30 mg thrice a day. In embodiments, latrepirdine is administered to the subject in a total daily dose of about 60 mg

In embodiments, the present disclosure provides a method of treating psychosis in a subject in need thereof, comprising administering oromucosally a therapeutically effective amount of latrepirdine or a pharmaceutically acceptable salt thereof to the subject.

The present disclosure provides a method of treating psychosis in a subject in need thereof, the method comprising administering to the subject a therapeutically effective amount of dexmedetomidine or a pharmaceutically acceptable salt thereof in combination with a therapeutically effective amount of latrepirdine or a pharmaceutically acceptable salt thereof.

The present disclosure provides a method of treating psychosis in a subject in need thereof, the method comprising administering oromucosally (e.g. sublingually, buccally or gingivally) to the subject a therapeutically effective amount of dexmedetomidine or a pharmaceutically acceptable salt thereof in combination with a therapeutically effective amount of latrepirdine or a pharmaceutically acceptable salt thereof.

In embodiments, the treatment is effective without causing significant sedation.

In embodiments, the treatment is effective without experiencing clinically significant cardiovascular effects. In embodiments, the severity of psychosis in the subject is assessed using PANS S scale.

The Positive and Negative Syndrome Scale (PANSS) standard has been widely used in clinical trials and is considered the “gold standard” for assessment of antipsychotic treatment efficacy. To assess a patient using PANSS, an approximately 45-minute clinical interview is conducted. The patient is rated from 1 to 7 on 30 different symptoms based on the interview as well as reports of family members or primary care hospital workers. Scores are often given separately for the positive items, negative items, and general psychopathology

In embodiments, the present disclosure provides a method of achieving a PANSS score reduction in psychosis for a sustained period of time in a subject comprising administering to the subject a therapeutically effective amount of latrepirdine or a pharmaceutically acceptable salt thereof.

In embodiments, the present disclosure provides a method of achieving a PANSS score reduction in psychosis for a sustained period of time in a subject comprising administering oromucosally to the subject a therapeutically effective amount of latrepirdine or a pharmaceutically acceptable salt thereof.

In embodiments, the present disclosure provides a method of achieving a PANS S score reduction in psychosis for a sustained period of time in a subject comprising administering to the subject a therapeutically effective amount of dexmedetomidine or a pharmaceutically acceptable salt thereof in combination with a therapeutically effective amount of latrepirdine or a pharmaceutically acceptable salt thereof.

In embodiments, the present disclosure provides a method of achieving a PANS S score reduction in psychosis for a sustained period of time in a subject comprising administering oromucosally to the subject a therapeutically effective amount of dexmedetomidine or a pharmaceutically acceptable salt thereof in combination with a therapeutically effective amount of latrepirdine or a pharmaceutically acceptable salt thereof.

In embodiments, the PANSS score reduction is at least about 20% to about 50% from baseline score. In embodiments, the PANSS score reduction is about 25% from baseline score. In embodiments, the PANSS total score reduction is about 30% from baseline score. In embodiments, the PANSS total score reduction is about 35% points from baseline score. In embodiments, the PANS S total score reduction is about 40% points from baseline score. In embodiments, the PANSS total score reduction is about 45% points from baseline score. In embodiments, the PANSS total score reduction is about 50% points from baseline score.

In embodiments, the psychosis is acute. In embodiments, the psychosis is chronic. In embodiments, the subject is agitated. In embodiments, the subject is non-agitated.

In embodiments, the psychosis is associated with a neuropsychiatric disorder selected from the group consisting of schizophrenia, schizoaffective disorder, depression, dementia and bipolar disorder or another related neuropsychiatric disorder. In embodiments, the psychosis is associated with neurodegenerative disorders.

In embodiments, the psychosis is associated with diseased condition such as substance abuse disorders (e.g., alcohol, opioid and other substance withdrawal).

In embodiments, the psychosis is acute. In embodiments, the psychosis is chronic. In embodiments, the psychosis is a single episode. In embodiments, the psychosis is recurring or includes recurrent episodes. In embodiments, the acute psychosis is associated with acute psychotic episodes and/or mixed episodes. In embodiments, dexmedetomidine and latrepirdine or pharmaceutically acceptable salts thereof are administered by the buccal route. In embodiments, dexmedetomidine and latrepirdine or pharmaceutically acceptable salts thereof are administered by the sublingual route. In embodiments, dexmedetomidine and latrepirdine or pharmaceutically acceptable salts thereof are administered sublingually or buccally in the form of a tablet or disc.

The combinations disclosed herein may be administered for as long as needed to treat agitation. In embodiments, said combination is administered at least once a day, such as once daily or twice daily, for at least 3 days, at least 5 days, at least 7 days, at least 10 days, at least 15 days, at least 30 days, at least 60 days, at least 90 days, at least 180 days, at least 365 days, or longer. The combinations disclosed herein may be administered for as long as needed to treat depression.

The unit doses may be administered once daily, twice daily, thrice daily or four times, five times, six times a day, preferably once, twice or thrice daily. The daily dose depends on the frequency of administration, preferably once or twice, or thrice or five times a day. The daily doses can be split into two, three, four, five or six times.

In embodiments, the present disclosure provides a combination comprising:
(i) a therapeutically effective amount of latrepirdine or a pharmaceutically acceptable salt thereof and
(ii) a therapeutically effective amount of dexmedetomidine or a pharmaceutically acceptable salt thereof,
for the treatment of behavioral and psychological symptoms in a subject in need thereof.

In embodiments, the present disclosure provides a synergistic combination comprising:
(i) a therapeutically effective amount of latrepirdine or a pharmaceutically acceptable salt thereof and
(ii) a therapeutically effective amount of dexmedetomidine or a pharmaceutically acceptable salt thereof,
for the treatment of agitation in a subject in need thereof.

In embodiments the therapeutically effective amount of dexmedetomidine or a pharmaceutically acceptable salt thereof is dexmedetomidine hydrochloride.

In embodiments the therapeutically effective amount of latrepirdine or a pharmaceutically acceptable salt thereof is latrepirdine hydrochloride or dihydrochloride.

In embodiments, the agitated subject has a baseline score in PEC scale of about 14 or higher.

In embodiments, the agitated subject experiences a PEC score reduction following administration of dosage forms of the present disclosure in accordance with the methods described herein. In embodiments, the patient achieves a change in PEC score of greater than −2 relative to baseline within 2 hours of administering the composition. For example, the PEC score reduction is about −1, about, about −2, about −3, about −4, about −5, about −6, about −7, about −8, about −9, or about −10 relative to baseline. In embodiments, the dosage form comprises dexmedetomidine or a pharmaceutically acceptable salt thereof. In embodiments, dexmedetomidine or a pharmaceutically acceptable salt thereof is present at a dose of about 0.5 micrograms to about 500 micrograms (e.g. about 30, about 60, about 80, about 90, about 120, about 180 or about 240 micrograms). In embodiments, the dosage form comprises latrepirdine or a pharmaceutically acceptable salt thereof. In embodiments, latrepirdine or a pharmaceutically acceptable salt thereof is present at a dose of about 0.5 mg to about 100 mg. In embodiments, the dosage form comprises dexmedetomidine or a pharmaceutically acceptable salt thereof and latrepirdine or a pharmaceutically acceptable salt thereof. In embodiments, the PEC score reduction is sustained for about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours following administration of the composition.

In embodiments, the agitated subject has a baseline score in ACES score of about 3 or below.

In embodiments, the agitated subject experiences an Agitation-Calmness Evaluation Scale (ACES) score improvement following administration of dosage forms of the present disclosure in accordance with the methods described herein. In embodiments, the agitation is reduced to a 2 (moderate agitation), 3 (mild agitation) or 4 (normal behavior) 2 hours after administering the composition, as measured by the Agitation-Calmness Evaluation Scale (ACES). For example, the ACES score is improved to about a 3 (mild agitation) or 4 (normal behavior). In embodiments, the dosage form comprises dexmedetomidine or a pharmaceutically acceptable salt thereof. In embodiments, dexmedetomidine or a pharmaceutically acceptable salt thereof is present at a dose of about 0.5 micrograms to about 500 micrograms (e.g. about 30, about 60, about 90, about 120, about 180 or about 240 micrograms). In embodiments, the dosage form comprises latrepirdine or a pharmaceutically acceptable salt thereof. In embodiments, latrepirdine or a pharmaceutically acceptable salt thereof is present at a dose of about 0.5 mg to about 500 mg. In embodiments, the dosage form comprises dexmedetomidine or a pharmaceutically acceptable salt thereof and latrepirdine or a pharmaceutically acceptable salt thereof. In embodiments, the ACES score improvement is sustained for about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours following administration of the composition. In embodiments, the ACES score after treatment is preferably between 3 and 7; for example, 3, 4, 5, 6, or 7. Advantageously, the improved ACES score is obtained shortly after latrepirdine (alone or, preferably, with dexmedetomidine) is administered; for example the improved ACES score may be obtained within about 2 hours of administering the composition. For example, the improved ACES score may be obtained within about 5 minutes, about 10 minutes, about 20 minutes, about 30 minutes, about 40 minutes, about 50 minutes, about 60 minutes, about 70 minutes, about 80 minutes, about 90 minutes, about 100 minutes, about 110 minutes, or about 120 minutes. The improved ACES score may be obtained within about 5 minutes to about 120 minutes, about 5 minutes to about 60 minutes, about 5 minutes to about 30 minutes, about 30 minutes to about 120 minutes, about 30 minutes to about 90 minutes, about 30 minutes to about 60 minutes, about 60 minutes to about 120 minutes, about 60 minutes to about 90 minutes, or about 90 minutes to about 120 minutes following administration of the composition.

In embodiments, the agitated subject has a baseline score in CGI-I of about 3 or higher.

In embodiments, the agitated subject experiences a CGI-I score improvement following administration of dosage forms of the present disclosure in accordance with the methods described herein. For example, the CGI-I score is improved to about a 1 (very much improved) or about a 2 (much improved). In embodiments, the dosage form comprises dexmedetomidine or a pharmaceutically acceptable salt thereof. In embodiments, dexmedetomidine or a pharmaceutically acceptable salt thereof is present at a dose of about 0.5 micrograms to about 500 micrograms (e.g. 30, 60, 90, 120, or 180 micrograms). In embodiments, the dosage form comprises latrepirdine or a pharmaceutically acceptable salt thereof. In embodiments, latrepirdine or a pharmaceutically acceptable salt thereof is present at a dose of about 0.5 mg to about 500 mg. In embodiments, latrepirdine or a pharmaceutically acceptable salt thereof is present at a dose of about 1 mg to about 100 mg. In embodiments, latrepirdine or a pharmaceutically acceptable salt thereof is present at a dose of about 5 mg to about 50 mg. In embodiments, the dosage form comprises dexmedetomidine or a pharmaceutically acceptable salt thereof and latrepirdine and a pharmaceutically acceptable salt thereof. In embodiments, the CGI-I score improvement is sustained for about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours following administration of the composition. In embodiments, the CGI-I score is about 1.

V. Medical Kits:

According to the present disclosure, there is provided a kit of parts comprising two oromucosal lyophilized tablet dosage forms (i) and (ii), the said dosage forms comprise:
(i) a therapeutically effective amount of dexmedetomidine or a pharmaceutically acceptable salt thereof;
(ii) a therapeutically effective amount of latrepirdine or a pharmaceutically acceptable salt thereof; and optionally
(iii) instructions for the simultaneous, sequential or separate administration of (i) and (ii) to a subject in need thereof.

According to the present disclosure, there is also provided a kit of parts comprising a single oromucosal lyophilized tablet dosage form comprising:
(i) a therapeutically effective amount of dexmedetomidine or a pharmaceutically acceptable salt thereof (e.g. hydrochloride salt);
(ii) a therapeutically effective amount of latrepirdine or a pharmaceutically acceptable salt thereof; and optionally
(iii) instructions for the administration of the single dosage form to a subject in need thereof.

Dexmedetomidine or a pharmaceutically acceptable salt thereof and latrepirdine or pharmaceutically acceptable salts thereof are each provided in a form that is suitable for administration in conjunction with the other.

In embodiments, dexmedetomidine and latrepirdine or pharmaceutically acceptable salts thereof are provided as a part of the same or single dosage form.

In embodiments, dexmedetomidine and latrepirdine or pharmaceutically acceptable salts thereof are provided as two separate dosage forms for administration (optionally repeatedly) of one dosage form prior to, after, and/or at the same time as administration with the other dosage form. When administered sequentially, the sequential administration may be close in time or remote in time. This may include situations where the two dosage forms are administered (optionally repeatedly) sufficiently closely in time for a beneficial effect for the patient that is greater over the course of the treatment of the relevant condition than if either of the two compositions are administered (optionally repeatedly) alone over the same course of treatment.

When used in the present context, the terms “administered simultaneously” and “administered at the same time” include individual doses of dexmedetomidine or a pharmaceutically acceptable salt thereof and latrepirdine or a pharmaceutically acceptable salt thereof are administered within about 24 hours, e.g. about 23 hours, about 22 hours, about 21 hours, about 20 hours, about 19 hours, about 18 hours, about 17 hours, about 16 hours, about 15 hours, about 14 hours, about 13 hours, about 12 hours, about 11 hours, about 10 hours, about 9 hours, about 8 hours, about 7 hours, about 6 hours, about 5 hours, about 4 hours, about 3 hours, about 2 hours, about 1 hours or less than about 1 hour (i.e. about 45 minutes, about 30 minutes, about 15 minutes, about 10 minutes, about 9 minutes, about 8 minutes, about 7 minutes, about 6 minutes, about 5 minutes, about 4 minutes, about 3 minutes, about 2 minutes or about 1 minute) of each other.

In embodiments, dexmedetomidine and latrepirdine or pharmaceutically acceptable salts thereof are administered at the same time. In embodiments, the active agents are administered together in the single dosage form. When dexmedetomidine and latrepirdine or pharmaceutically acceptable salts thereof are provided as separate dosage forms, each dosage form may be packaged separately for use in conjunction with the other in combination therapy. Alternatively, the two dosage forms may be packaged and presented together as separate components of a “combination pack” for use in conjunction with each other in combination therapy.

### SPECIFIC EMBODIMENTS

Embodiment 1. A method of treating agitation in an agitated subject, comprising administering to the subject a therapeutic amount of latrepirdine or a pharmaceutically acceptable salt thereof, in combination with a therapeutic amount of dexmedetomidine or a pharmaceutically acceptable salt thereof.

Embodiment 2. A method of treating agitation in an agitated subject, comprising oromucosally administering to the subject a dosage form comprising a therapeutically effective amount of latrepirdine or a pharmaceutically acceptable salt thereof.

Embodiment 3. A method of treating agitation in an agitated subject, comprising administering to the subject a therapeutically effective amount of latrepirdine or a pharmaceutically acceptable salt thereof.

Embodiment 4. A method of treating agitation in an agitated subject, comprising oromucosally administering to the subject a dosage form comprising:
(i) a therapeutically effective amount of latrepirdine a pharmaceutically acceptable salt thereof and
(ii) a therapeutically effective amount of dexmedetomidine or a pharmaceutically acceptable salt thereof.

Embodiment 5. A method of reducing noradrenergic hyperarousal, comprising oromucosally administering a dosage form comprising a therapeutically effective amount of latrepirdine or a pharmaceutically acceptable salt thereof to a subject.

Embodiment 6. A method of reducing noradrenergic hyperarousal, comprising oromucosally administering a dosage form comprising a therapeutically effective amount of latrepirdine or a pharmaceutically acceptable salt and a therapeutically effective amount of dexmedetomidine or a pharmaceutically acceptable salt thereof.

Embodiment 7. A method of treatment of behavioral and psychological symptoms in subjects with a neurodegenerative disorder, comprising oromucosally administering therapeutically effective amount of latrepirdine or a pharmaceutically acceptable salt thereof.

Embodiment 8. A method of treatment of behavioral and psychological symptoms in subjects with a neurodegenerative disorder, comprising oromucosally administering therapeutically effective amounts of latrepirdine and dexmedetomidine or pharmaceutically acceptable salts thereof.

Embodiment 9. A method of treatment of behavioral and psychological symptoms in subjects with a neuropsychiatric disorder, comprising oromucosally administering therapeutically effective amount of latrepirdine or a pharmaceutically acceptable salt thereof.

Embodiment 10. A method of treatment of behavioral and psychological symptoms in subjects with a neuropsychiatric disorder, comprising oromucosally administering therapeutically effective amounts of latrepirdine or a pharmaceutically acceptable salt and dexmedetomidine or pharmaceutically acceptable salts thereof.

Embodiment 11. Use of therapeutically effective amount of latrepirdine or a pharmaceutically acceptable salt thereof for the treatment of behavioral and psychological symptoms in subjects with a neurodegenerative disorder.

Embodiment 12. Use of therapeutically effective amount of latrepirdine or a pharmaceutically acceptable salt thereof for the treatment of behavioral and psychological symptoms in subjects with a neuropsychiatric disorder.

Embodiment 13. Use of therapeutically effective amount of latrepirdine or a pharmaceutically acceptable salt thereof and dexmedetomidine or pharmaceutically acceptable salt thereof for the treatment of behavioral and psychological symptoms in subjects with a neurodegenerative disorder.

Embodiment 14. Use of therapeutically effective amount of latrepirdine or a pharmaceutically acceptable salt thereof and dexmedetomidine or pharmaceutically acceptable salt thereof for the treatment of behavioral and psychological symptoms in subjects with a neuropsychiatric disorder.

Embodiment 15. A method of treatment of agitation in a subject comprising oromucosally administering therapeutically effective amount of latrepirdine or a pharmaceutically acceptable salt thereof, wherein the subject has a concomitant Alzheimer's disease.

Embodiment 16. A method of treatment of chronic agitation in a subject comprising oromucosally administering therapeutically effective amount of latrepirdine or a pharmaceutically acceptable salt thereof, wherein the subject has a concomitant dementia.

Embodiment 17. A method of treatment of agitation in a subject comprising oromucosally administering therapeutically effective amount of latrepirdine or a pharmaceutically acceptable salt thereof, wherein the subject has a concomitant Parkinson's disease.

Embodiment 18. A method of treatment of agitation in a subject comprising oromucosally administering therapeutically effective amount of latrepirdine or a pharmaceutically acceptable salt thereof, wherein the subject has a concomitant PTSD.

Embodiment 19. A method of treatment of agitation in a subject comprising oromucosally administering therapeutically effective amount of latrepirdine or a pharmaceutically acceptable salt thereof, wherein the subject has a concomitant vascular cognitive impairment.

Embodiment 20. A method of treatment of agitation in a subject comprising oromucosally administering therapeutically effective amount of latrepirdine or a pharmaceutically acceptable salt thereof, wherein the subject has a concomitant Huntington's disease.

Embodiment 21. A method of treatment of agitation in a subject comprising oromucosally administering therapeutically effective amount of latrepirdine or a pharmaceutically acceptable salt thereof, wherein the subject has a concomitant schizophrenia.

Embodiment 22. A method of treatment of agitation in a subject comprising oromucosally administering therapeutically effective amount of latrepirdine or a pharmaceutically acceptable salt thereof, wherein the subject has a concomitant bipolar disorder.

Embodiment 23. A method of treatment of agitation in a subject comprising oromucosally administering therapeutically effective amount of latrepirdine or a pharmaceutically acceptable salt thereof, wherein the subject has a concomitant depression.

Embodiment 24. A method of treatment of agitation in a subject comprising oromucosally administering therapeutically effective amount of latrepirdine or a pharmaceutically acceptable salt thereof, wherein the subject has a concomitant delirium.

Embodiment 25. A method of treatment of agitation in a subject comprising oromucosally administering therapeutically effective amount of latrepirdine or a pharmaceutically acceptable salt thereof in combination with dexmedetomidine or pharmaceutically acceptable salt thereof, wherein the subject has a concomitant Alzheimer's disease.

Embodiment 26. A method of treatment of chronic agitation in a subject comprising oromucosally administering therapeutically effective amount of latrepirdine or a pharmaceutically acceptable salt thereof in combination with dexmedetomidine or pharmaceutically acceptable salt thereof, wherein the subject has a concomitant dementia.

Embodiment 27. A method of treatment of agitation in a subject comprising oromucosally administering therapeutically effective amount of latrepirdine or a pharmaceutically acceptable salt thereof in combination with dexmedetomidine or pharmaceutically acceptable salt thereof, wherein the subject has a concomitant Parkinson's disease.

Embodiment 28. A method of treatment of agitation in a subject comprising oromucosally administering therapeutically effective amount of latrepirdine or a pharmaceutically acceptable salt thereof in combination with dexmedetomidine or pharmaceutically acceptable salt thereof, wherein the subject has a concomitant PTSD.

Embodiment 29. A method of treatment of agitation in a subject comprising oromucosally administering therapeutically effective amount of latrepirdine or a pharmaceutically acceptable salt thereof in combination with dexmedetomidine or pharmaceutically acceptable salt thereof, wherein the subject has a concomitant Huntington's disease.

Embodiment 29. A method of treatment of agitation in a subject comprising oromucosally administering a therapeutically effective amount of latrepirdine or a pharmaceutically acceptable salt thereof in combination with dexmedetomidine or pharmaceutically acceptable salt thereof, wherein the subject has a concomitant schizophrenia.

Embodiment 30. A method of treatment of agitation in a subject comprising oromucosally administering a therapeutically effective amount of latrepirdine or a pharmaceutically acceptable salt thereof in combination with dexmedetomidine or pharmaceutically acceptable salt thereof, wherein the subject has a concomitant bipolar disorder.

Embodiment 31. A method of treatment of agitation in a subject comprising oromucosally administering a therapeutically effective amount of latrepirdine or a pharmaceutically acceptable salt thereof in combination with dexmedetomidine or pharmaceutically acceptable salt thereof, wherein the subject has a concomitant depression.

Embodiment 32. A method of treatment of agitation in a subject comprising oromucosally administering a therapeutically effective amount of latrepirdine or a pharmaceutically acceptable salt thereof in combination with dexmedetomidine or pharmaceutically acceptable salt thereof, wherein the subject has a concomitant vascular cognitive impairment.

Embodiment 33. A method of treatment of agitation in a subject comprising oromucosally administering a therapeutically effective amount of latrepirdine or a pharmaceutically acceptable salt thereof in combination with dexmedetomidine or pharmaceutically acceptable salt thereof, wherein the subject has a concomitant delirium.

Embodiment 34. The method according to any of preceding embodiments, wherein the agitation is severe.

Embodiment 35. The method according to any of preceding embodiments, wherein the agitation is mild or moderate.

Embodiment 36. The method according to any of preceding embodiments, wherein latrepirdine is administered in a dosage amount of about 10 mg once a day.

Embodiment 37. The method according to any of preceding embodiments, wherein latrepirdine is administered in a dosage amount of about 20 mg once a day.

Embodiment 38. The method according to any of preceding embodiments, wherein latrepirdine is administered in a dosage amount of about 10 mg twice a day.

Embodiment 39. The method according to any of preceding embodiments, wherein latrepirdine is administered in a dosage amount of about 10 mg thrice a day.

Embodiment 40. The method according to any of preceding embodiments, wherein latrepirdine is administered in a dosage amount of about 20 mg twice a day.

Embodiment 41. The method according to any of preceding embodiments, wherein latrepirdine is administered to the subject in a total daily dose of about 60 mg.

Embodiment 42. A method of treating agitation in an agitated subject, comprising administering about 0.1 mg to about 500 mg of latrepirdine or a pharmaceutically acceptable salt thereof and about 0.5 micrograms to about 300 micrograms of dexmedetomidine or a pharmaceutically acceptable salt thereof to the subject.

Embodiment 42. A method of treating agitation in an agitated subject, comprising administering oromucosally about 0.1 mg to about 100 mg of latrepirdine or a pharmaceutically acceptable salt thereof and about 10 micrograms to about 300 micrograms of dexmedetomidine or a pharmaceutically acceptable salt thereof to the subject.

Embodiment 43. A method of treating agitation in an agitated subject, comprising oromucosally administering about 5 mg to about 60 mg of latrepirdine or a pharmaceutically acceptable salt thereof and about 20 micrograms to about 300 micrograms of dexmedetomidine or a pharmaceutically acceptable salt thereof to the subject.

Embodiment 44. A method of treating agitation in an agitated subject, comprising administering oromucosally about 1 mg to about 40 mg of latrepirdine or a pharmaceutically acceptable salt thereof and about 10 micrograms to about 200 micrograms of dexmedetomidine or a pharmaceutically acceptable salt thereof to the subject.

Embodiment 45. A method of treating agitation in an agitated subject, comprising administering about 10 mg of latrepirdine or a pharmaceutically acceptable salt thereof and about 30 micrograms of dexmedetomidine or a pharmaceutically acceptable salt thereof to the subject.

Embodiment 46. A method of treating agitation in an agitated subject, comprising administering about 10 mg of latrepirdine or a pharmaceutically acceptable salt thereof and about 30 micrograms of dexmedetomidine or a pharmaceutically acceptable salt thereof to the subject, wherein the agitation is caused by noradrenergic hyperarousal.

Embodiment 47. A method of treating agitation in an agitated subject, comprising administering about 20 mg of latrepirdine or a pharmaceutically acceptable salt thereof and about 30 micrograms of dexmedetomidine or a pharmaceutically acceptable salt thereof to the subject.

Embodiment 48. A method of treating agitation in an agitated subject, comprising administering about 20 mg of latrepirdine or a pharmaceutically acceptable salt thereof and about 30 micrograms of dexmedetomidine or a pharmaceutically acceptable salt thereof to the subject, wherein the agitation is caused by noradrenergic hyperarousal.

Embodiment 49. A method of treating agitation in an agitated subject, comprising administering about 10 mg of latrepirdine or a pharmaceutically acceptable salt thereof and about 40 micrograms of dexmedetomidine or a pharmaceutically acceptable salt thereof to the subject,

Embodiment 50. A method of treating agitation in an agitated subject, comprising administering about 10 mg of latrepirdine or a pharmaceutically acceptable salt thereof and about 40 micrograms of dexmedetomidine or a pharmaceutically acceptable salt thereof to the subject, wherein the agitation is caused by noradrenergic hyperarousal.

Embodiment 51. A method of treating agitation in an agitated subject, comprising administering about 20 mg of latrepirdine or a pharmaceutically acceptable salt thereof and about 40 micrograms of dexmedetomidine or a pharmaceutically acceptable salt thereof to the subject.

Embodiment 52. A method of treating agitation in an agitated subject, comprising administering about 20 mg of latrepirdine or a pharmaceutically acceptable salt thereof and about 40 micrograms of dexmedetomidine or a pharmaceutically acceptable salt thereof to the subject, wherein the agitation is caused by noradrenergic hyperarousal.

Embodiment 53. A method of treating agitation in an agitated subject, comprising administering about 10 mg of latrepirdine or a pharmaceutically acceptable salt thereof and about 60 micrograms of dexmedetomidine or a pharmaceutically acceptable salt thereof to the subject.

Embodiment 54. A method of treating agitation in an agitated subject, comprising administering about 10 mg of latrepirdine or a pharmaceutically acceptable salt thereof and about 60 micrograms of dexmedetomidine or a pharmaceutically acceptable salt thereof to the subject, wherein the agitation is caused by noradrenergic hyperarousal.

Embodiment 55. A method of treating agitation in an agitated subject, comprising administering about 20 mg of latrepirdine or a pharmaceutically acceptable salt thereof and about 80 micrograms of dexmedetomidine or a pharmaceutically acceptable salt thereof to the subject.

Embodiment 56. A method of treating agitation in an agitated subject, comprising administering about 20 mg of latrepirdine or a pharmaceutically acceptable salt thereof and about 80 micrograms of dexmedetomidine or a pharmaceutically acceptable salt thereof to the subject, wherein the agitation is caused by noradrenergic hyperarousal.

Embodiment 57. A method of treating agitation in an agitated subject, comprising administering about 10 mg of latrepirdine or a pharmaceutically acceptable salt thereof and about 120 micrograms of dexmedetomidine or a pharmaceutically acceptable salt thereof to the subject.

Embodiment 58. A method of treating agitation in an agitated subject, comprising administering about 10 mg of latrepirdine or a pharmaceutically acceptable salt thereof and about 120 micrograms of dexmedetomidine or a pharmaceutically acceptable salt thereof to the subject, wherein the agitation is caused by noradrenergic hyperarousal.

Embodiment 59. A method of treating agitation in an agitated subject, comprising administering about 20 mg of latrepirdine or a pharmaceutically acceptable salt thereof and about 120 micrograms of dexmedetomidine or a pharmaceutically acceptable salt thereof to the subject.

Embodiment 60. A method of treating agitation in an agitated subject, comprising administering about 20 mg of latrepirdine or a pharmaceutically acceptable salt thereof and about 120 micrograms of dexmedetomidine or a pharmaceutically acceptable salt thereof to the subject, wherein the agitation is caused by noradrenergic hyperarousal.

Embodiment 61. A method of treating agitation in an agitated subject, comprising administering about 10 mg of latrepirdine or a pharmaceutically acceptable salt thereof and about 180 micrograms of dexmedetomidine or a pharmaceutically acceptable salt thereof to the subject.

Embodiment 62. A method of treating agitation in an agitated subject, comprising administering about 10 mg of latrepirdine or a pharmaceutically acceptable salt thereof and about 180 micrograms of dexmedetomidine or a pharmaceutically acceptable salt thereof to the subject, wherein the agitation is caused by noradrenergic hyperarousal.

Embodiment 63. A method of treating agitation in an agitated subject, comprising administering about 20 mg of latrepirdine or a pharmaceutically acceptable salt thereof and about 180 micrograms of dexmedetomidine or a pharmaceutically acceptable salt thereof to the subject.

Embodiment 64. A method of treating agitation in an agitated subject, comprising administering about 20 mg of latrepirdine or a pharmaceutically acceptable salt thereof and about 180 micrograms of dexmedetomidine or a pharmaceutically acceptable salt thereof to the subject, wherein the agitation is caused by noradrenergic hyperarousal.

Embodiment 65. A method of treating depression in a subject in need thereof, comprising administering oromucosally to the subject a dosage form comprising a therapeutically effective amount of latrepirdine or a pharmaceutically acceptable salt thereof.

Embodiment 66. A method of treating depression in a subject in need thereof, the method comprising administering oromucosally to the subject a dosage form comprising a therapeutically effective amount of dexmedetomidine or a pharmaceutically acceptable salt thereof.

Embodiment 67. A method of treating depression in a subject in need thereof, the method comprising administering to the subject a dosage form comprising therapeutically effective amount of dexmedetomidine or a pharmaceutically acceptable salt thereof in combination with a therapeutically effective amount of latrepirdine or a pharmaceutically acceptable salt thereof.

Embodiment 68. A method of treating depression in a subject in need thereof, the method comprising administering oromucosally to the subject a dosage form comprising therapeutically effective amount of dexmedetomidine or a pharmaceutically acceptable salt thereof in combination with a therapeutically effective amount of latrepirdine or a pharmaceutically acceptable salt thereof.

Embodiment 69. A method of treating depression in a subject in need thereof, the method comprising administering oromucosally to the subject about 5 mg to about 100 mg of latrepirdine or a pharmaceutically acceptable salt thereof and about 10 micrograms to about 300 micrograms of dexmedetomidine or a pharmaceutically acceptable salt thereof

Embodiment 70. The method according to embodiments 65 to 69, wherein the depression is moderate or severe.

Embodiment 71. The method according to embodiments 65 to 69, wherein the depression is major depression.

Embodiment 72. The method according to embodiments 65 to 69, wherein the depression is mixed depression.

Embodiment 73. A method of treating depression in a subject in need thereof, the method comprising administering oromucosally to the subject about 10 mg of latrepirdine or a pharmaceutically acceptable salt thereof and about 30 micrograms of dexmedetomidine or a pharmaceutically acceptable salt thereof

Embodiment 74. A method of treating depression in a subject in need thereof, the method comprising administering oromucosally to the subject about 10 mg of latrepirdine or a pharmaceutically acceptable salt thereof and about 40 micrograms of dexmedetomidine or a pharmaceutically acceptable salt thereof

Embodiment 75. A method of treating depression in a subject in need thereof, the method comprising administering oromucosally to the subject about 10 mg of latrepirdine or a pharmaceutically acceptable salt thereof and about 60 micrograms of dexmedetomidine or a pharmaceutically acceptable salt thereof

Embodiment 76. A method of treating depression in a subject in need thereof, the method comprising administering oromucosally to the subject about 20 mg of latrepirdine or a pharmaceutically acceptable salt thereof and about 30 micrograms of dexmedetomidine or a pharmaceutically acceptable salt thereof.

Embodiment 77. A method of treating depression in a subject in need thereof, the method comprising administering oromucosally to the subject about 20 mg of latrepirdine or a pharmaceutically acceptable salt thereof and about 40 micrograms of dexmedetomidine or a pharmaceutically acceptable salt thereof.

Embodiment 78. A method of treating depression in a subject in need thereof, the method comprising administering oromucosally to the subject about 10 mg of latrepirdine or a pharmaceutically acceptable salt thereof and about 60 micrograms of dexmedetomidine or a pharmaceutically acceptable salt thereof.

Embodiment 79. A method of treating depression in a subject in need thereof, the method comprising administering oromucosally to the subject about 20 mg of latrepirdine or a pharmaceutically acceptable salt thereof and about 60 micrograms of dexmedetomidine or a pharmaceutically acceptable salt thereof.

Embodiment 80. A method of treating depression in a subject in need thereof, the method comprising administering oromucosally to the subject about 10 mg of latrepirdine or a pharmaceutically acceptable salt thereof and about 80 micrograms of dexmedetomidine or a pharmaceutically acceptable salt thereof.

Embodiment 81. A method of treating depression in a subject in need thereof, the method comprising administering oromucosally to the subject about 20 mg of latrepirdine or a pharmaceutically acceptable salt thereof and about 80 micrograms of dexmedetomidine or a pharmaceutically acceptable salt thereof.

Embodiment 82. A method of reducing score on HDRS scale in a human subject suffering from depression comprising administering oromucosally effective amounts of dexmedetomidine either alone or in combination with latrepirdine or pharmaceutically acceptable salts thereof.

Embodiment 83. A method of reducing score on MADRS scale in a human subject suffering from depression comprising administering oromucosally effective amounts of dexmedetomidine either alone or in combination with latrepirdine or pharmaceutically acceptable salts thereof.

Embodiment 84. The method according to any of Embodiments 1, 4, 6, 8, 10, 13, 14, 25-33, 42-64, 66-69, 73-78, wherein dexmedetomidine or a pharmaceutically acceptable salt thereof (e.g. hydrochloride salt) and latrepirdine are administered concurrently or sequentially in two separate dosage forms.

Embodiment 85. The method according to any of Embodiments 1, 4, 6, 8, 10, 13, 14, 25-33, 42-64, 66-69, 73-78, wherein dexmedetomidine or a pharmaceutically acceptable salt thereof and latrepirdine are administered simultaneously in a single unit dosage form.

Embodiment 86. The method according to Embodiment 85, wherein the two separate dosage forms are sequentially administered separated by a specific period of time such as 1, 2, 3, 4, 5, 6, 7, 8, 9, 10, 11, 12, 13, 14, 15, 20, 25, 30, 35, 40, 45, 50, 55 or 60 minutes, or 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours.

Embodiment 87. The method according to any of the preceding embodiments, wherein the dosage form is lyophilized.

Embodiment 88. The method according to any of any of the preceding embodiments, wherein said dosage form is administered sublingually or buccally.

Embodiments 89. The method according to any of the preceding embodiments, wherein the dosage form disintegrates in not less than about 1 minute upon contact with an oral mucosa.

Embodiment 90. The method according to any of the preceding embodiments, wherein the dosage form is mucoadhesive.

Embodiment 91. The method according to Embodiment 88, wherein dexmedetomidine and latrepirdine or pharmaceutically acceptable salts thereof are administered oromucosally in a dosage form selected from the group consisting of a film, wafer, patch, lozenge, gel, spray, tablet and liquid drops.

Embodiment 92. The method according to Embodiment 91, wherein said dexmedetomidine and latrepirdine or pharmaceutically acceptable salts thereof are administered sublingually or buccally in the form of a tablet.

Embodiment 93. The method according to any of the preceding embodiments, wherein the agitation is associated with neurodegenerative disorder selected from the group consisting of Alzheimer disease, frontotemporal dementia (FTD), dementia, dementia with Lewy bodies (DLB), post-traumatic stress disorder, Parkinson's disease, vascular dementia, vascular cognitive impairment, Huntington's disease, multiple sclerosis, Creutzfeldt-Jakob disease, multiple system atrophy, and progressive supranuclear palsy.

Embodiment 94. The method according to any of the preceding embodiments, wherein the agitation is associated with a neuropsychiatric disorder selected from the group consisting of schizophrenia, bipolar disorder, bipolar mania, delirium, and depression.

Embodiment 95. The method according to any of the preceding embodiments, wherein the agitation is associated with alcohol withdrawal, opioid use disorder, opioid withdrawal and substance abuse withdrawal.

Embodiment 96. The method according to any of the preceding embodiments, wherein the agitation is associated with an OPD/IPD procedure (e.g. MRI, CT or CAT scan, lumbar puncture, bone marrow aspiration/biopsy, tooth extraction or other dental procedures).

Embodiment 97. The method according to any of the preceding embodiments, wherein the agitation is acute.

Embodiment 98. The method according to any of the preceding embodiments, wherein the agitation is chronic.

Embodiment 99: The method according to any of the preceding embodiments, wherein the agitation is caused by noradrenergic hyperarousal.

Embodiment 100. The method according to any of the preceding embodiments, wherein the agitation is treated without causing significant sedation.

Embodiment 101. The method according to any of the preceding embodiments, wherein latrepirdine or a pharmaceutically acceptable salt thereof is administered in an amount of about 0.5 mg to about 500 mg.

Embodiment 102. The method according to Embodiment 101, wherein said latrepirdine or a pharmaceutically acceptable salt thereof is administered in an amount of about 10 mg to about 100 mg.

Embodiment 103. The method according to any of the preceding embodiments, wherein said dexmedetomidine is present in an amount of about 0.5 micrograms to about 300 micrograms.

Embodiment 104. The method according to Embodiment 103, wherein said dexmedetomidine is present in an amount of about 10 micrograms to about 300 micrograms.

Embodiment 105. The method according to any of preceding embodiments, wherein the dosage form(s) is administered multiple times a day.

Embodiment 106. The method according to any of preceding embodiments, wherein the dosage form(s) is administered once a day.

Embodiment 107. The method according to any of the preceding embodiments, wherein dexmedetomidine or a pharmaceutically acceptable salt thereof is dexmedetomidine hydrochloride.

Embodiment 108. The method according to any of the preceding embodiments, wherein latrepirdine or a pharmaceutically acceptable salt thereof is latrepirdine hydrochloride (or dihydrochloride).

Embodiment 109. An oromucosal dosage form comprising:
(i) a therapeutically effective amount of latrepirdine or a pharmaceutically acceptable salt thereof;
(ii) one or more mucoadhesive agents and
(iii) one or more pharmaceutically acceptable excipients or carriers;
wherein the dosage form disintegrates in not less than about 1 minute upon contact with an oral mucosa.

Embodiment 110. An oromucosal dosage form comprising:
(i) a therapeutically effective amount of dexmedetomidine or a pharmaceutically acceptable salt thereof;
(ii) one or more mucoadhesive agents and
(iii) one or more pharmaceutically acceptable excipients or carriers;
wherein the dosage form disintegrates in not less than about 1 minute upon contact with an oral mucosa.

Embodiment 111. An oromucosal dosage form comprising:
(iv) a therapeutically effective amount of latrepirdine or a pharmaceutically acceptable salt thereof;
(v) a therapeutically effective amount of dexmedetomidine or a pharmaceutically acceptable salt thereof;
(vi) (iii) one or more mucoadhesive agents and
(vii) one or more pharmaceutically acceptable excipients or carriers;
wherein the dosage form disintegrates in not less than about 1 minute upon contact with an oral mucosa.

Embodiment 112. The oromucosal dosage form according to Embodiment 109 or 110, where dexmedetomidine or a pharmaceutically acceptable salt thereof is dexmedetomidine hydrochloride.

Embodiment 113. The oromucosal dosage form according to any of Embodiments 109 to 111, wherein latrepirdine or a pharmaceutically acceptable salt thereof is latrepirdine hydrochloride or dihydrochloride.

Embodiment 114. The oromucosal dosage form according to Embodiment 110 or 111, wherein said therapeutic amount of dexmedetomidine or a pharmaceutically acceptable salt thereof (e.g. the hydrochloride) is from about 5 micrograms to about 300 micrograms per unit dose, e.g. about 20 micrograms to about 200 micrograms per unit dose, about 30 micrograms to about 100 micrograms per unit dose, or about 10 micrograms to about 50 micrograms per unit dose.

Embodiment 115. The oromucosal dosage form according to Embodiment 114, wherein the therapeutic amount of dexmedetomidine or a pharmaceutically acceptable salt thereof is about 30 micrograms, about 60 micrograms, about 90 micrograms, about 120 micrograms, or about 180 micrograms.

Embodiment 116. The oromucosal dosage form according to Embodiments 109 to 111, wherein therapeutic amount of latrepirdine or a pharmaceutically acceptable salt thereof is from about 0.5 mg to about 100 mg per unit dose.

Embodiment 117. The oromucosal dosage form according to Embodiments 109 to 111, wherein therapeutic amount of latrepirdine or a pharmaceutically acceptable salt thereof is from about 10 mg to about 100 mg per unit dose.

Embodiment 118. The oromucosal dosage form according to Embodiments 109 to 111, wherein the one or more mucoadhesive agents are selected from the group consisting of polyacrylic acid polymers, methacrylic acid polymers, cellulose derivatives such as hydroxyethyl cellulose, (HEC), hydroxypropyl cellulose (HPC), ethyl hydroxyethyl cellulose, hydroxypropyl methylcellulose (HPMC), methyl cellulose, sodium carboxymethyl cellulose, thiolated carboxymethyl cellulose; polysaccharides, xanthan gum, karaya gum, tragacanth gum, propylene glycol, propylene glycol alginate, sodium alginate, polyethylene oxide, microcrystalline cellulose (Avicel), croscarmellose and mixtures thereof.

Embodiment 119. The oromucosal dosage form according to Embodiment 118, wherein said one or more mucoadhesive agents are present in an amount of about 0.5% to about 20% w/w.

Embodiment 120. The oromucosal dosage form according to Embodiment 118, wherein the polyacrylic acid polymer is carbomer, polycarbophil, or a combination thereof.

Embodiment 121. The oromucosal dosage form according to Embodiment 118, wherein the polysaccharides are pectin, chitosan, or a combination thereof.

Embodiment 122. The oromucosal dosage form according to Embodiments 109 to 111, wherein one or more of the pharmaceutically acceptable excipients or carriers are selected from the group consisting of disintegrants, fillers/diluents (matrix forming agents), binders, glidants, lubricants, plasticizers, pH regulators, coloring agents, flavoring agents, taste masking agents, viscosity enhancers, sweetening agents and combinations thereof.

Embodiment 123. The oromucosal dosage form according to any of the preceding embodiments, wherein the dosage form has a mucoadhesive peak force greater than about 50 g (e.g. about 100 g, about 200 g, about 300 g, about 400 g, about 500 g, about 600 g, about 700 g, about 800 g, about 900 g, about 1000 g, about 1100 g, about 1200 g, about 1300 g, about 1400 g or about 1500 g).

Embodiments 124. The oromucosal dosage form according to any of the preceding embodiments, wherein the dosage form is prepared by spray drying, sublimation, nanonization, granulation, direct compression or lyophilization, preferably lyophilization.

Embodiment 125. The oromucosal dosage form according to any of the preceding embodiments, wherein the dosage form is a sublingual tablet.

Embodiment 126. The oromucosal dosage form according to any of the preceding embodiments, wherein the dosage form is a buccal tablet.

Embodiment 127. An oromucosal (e.g. sublingual or buccal) lyophilized tablet comprising: (i) a therapeutically effective amount of latrepirdine or a pharmaceutically acceptable salt thereof; (ii) xanthan gum (iii) croscarmellose sodium (or) sodium starch glycolate; (iv) sucralose; (v) magnesium stearate and)/or silicon dioxide; (vi) lactose monohydrate (or) mannitol and optionally other pharmaceutical acceptable excipients; wherein the tablet disintegrates in not less than about 1 minute upon contact with an oral mucosa.

Embodiment 128. An oromucosal (e.g. sublingual or buccal) lyophilized tablet comprising: (i) a therapeutically effective amount of latrepirdine or a pharmaceutically acceptable salt thereof; (ii) sodium alginate (iii) croscarmellose sodium (or) sodium starch glycolate; (iv) sucralose; (v) magnesium stearate and/or silicon dioxide; (vi) lactose monohydrate or mannitol and optionally other pharmaceutical acceptable excipients; wherein the tablet disintegrates in not less than about 1 minute upon contact with an oral mucosa.

Embodiment 129. An oromucosal (e.g. sublingual or buccal) lyophilized tablet comprising: (i) a therapeutically effective amount of latrepirdine or a pharmaceutically acceptable salt thereof; (ii) carbomer (iii) croscarmellose sodium or sodium starch glycolate; (iv) sucralose; (v) magnesium stearate and/or silicon dioxide; (vi) lactose monohydrate or mannitol and optionally other pharmaceutical acceptable excipients; wherein the tablet disintegrates in not less than about 1 minute upon contact with an oral mucosa.

Embodiment 130. An oromucosal (e.g. sublingual or buccal) lyophilized tablet comprising: (i) a therapeutically effective amount of latrepirdine or a pharmaceutically acceptable salt thereof; (ii) a therapeutically effective amount of dexmedetomidine or a pharmaceutically acceptable salt thereof; (iii) carbomer, hydroxypropyl cellulose, hydroxypropyl methylcellulose, or polyethylene oxide; (iv) croscarmellose sodium, or sodium starch glycolate; (v) sucralose; (vi) magnesium stearate and/or silicon dioxide; (vii) lactose or mannitol; and (viii) optionally other pharmaceutical acceptable excipients; wherein the tablet disintegrates in not less than about 1 minute upon contact with an oral mucosa.

Embodiment 131. An oromucosal (e.g. sublingual or buccal) lyophilized tablet comprising (i) a therapeutically effective amount of latrepirdine or a pharmaceutically acceptable salt thereof; (ii) a therapeutically effective amount of dexmedetomidine or a pharmaceutically acceptable salt thereof; (iii) sodium alginate, xanthan gum, carbomer, hydroxypropyl cellulose, hydroxypropyl methylcellulose, or polyethylene oxide; (iv) croscarmellose sodium or sodium starch glycolate; (v) sucralose; (vi) magnesium stearate and/or silicon dioxide; (vii) lactose or mannitol; and (viii) optionally other pharmaceutical acceptable excipients; wherein the tablet disintegrates in not less than about 1 minute upon contact with an oral mucosa.

Embodiment 132. The oromucosal lyophilized tablet according to Embodiments 127 to 131, wherein the therapeutically effective amount of dexmedetomidine or a pharmaceutically acceptable salt thereof is about 10 micrograms to about 300 micrograms.

Embodiment 133. The oromucosal lyophilized tablet according to Embodiment 132, wherein therapeutically effective amount of dexmedetomidine or a pharmaceutically acceptable salt thereof is about 30 micrograms, about 60 micrograms, about 90 micrograms, about 120 micrograms, about 180 micrograms or about 240 micrograms.

Embodiment 134. The oromucosal lyophilized tablet according to Embodiments 127 to 131, wherein therapeutically effective amount of latrepirdine or a pharmaceutically acceptable salt thereof is about 10 mg to about 100 mg.

Embodiment 135. The oromucosal lyophilized tablet according to Embodiment 133, wherein therapeutically effective amount of latrepirdine or a pharmaceutically acceptable salt thereof is about 30 mg.

Embodiment 136. The oromucosal dosage form according to any one of preceding embodiments, administered for the treatment of agitation caused by noradrenergic hyperarousal.

Embodiment 137. The oromucosal dosage form according to Embodiment 136, wherein the agitation caused by noradrenergic hyperarousal is associated with neurodegenerative disorders selected from a group consisting of dementia, Alzheimer's disease, frontotemporal dementia, Parkinsonism, sundown syndrome in Alzheimer's disease/dementia, or other neurodegenerative disorders.

Embodiment 138. The oromucosal dosage form according to Embodiment 136, wherein the agitation caused by noradrenergic hyperarousal is associated with neuropsychiatric disorders selected from a group consisting of schizophrenia, bipolar disorder, bipolar mania, delirium, depression, or another related neuropsychiatric disorder.

Embodiment 139. The oromucosal dosage form according to embodiment 136, wherein the agitation caused by noradrenergic hyperarousal is associated with alcohol withdrawal, opioid use disorder, opioid withdrawal and substance abuse withdrawal.

Embodiment 140. The oromucosal dosage form according to any of the preceding embodiments, wherein dexmedetomidine and latrepirdine or pharmaceutically acceptable salts thereof are provided as a single unit dosage form or in separate dosage forms.

Embodiment 141. A pharmaceutical combination for treating agitation caused by noradrenergic hyperarousal, comprising a therapeutic amount of dexmedetomidine, or a pharmaceutically acceptable salt thereof (e.g. hydrochloride salt) and a therapeutic amount of latrepirdine or a pharmaceutically acceptable salt thereof.

Embodiment 142. The combination according to Embodiment 141, wherein dexmedetomidine or a pharmaceutically acceptable salt thereof is dexmedetomidine hydrochloride.

Embodiment 143. The combination according to Embodiment 141, wherein latrepirdine or a pharmaceutically acceptable salt thereof is latrepirdine hydrochloride.

Embodiment 144. The combination according to embodiment 141, wherein the therapeutic amount of dexmedetomidine or a pharmaceutically acceptable salt thereof includes from about 5 micrograms to about 300 micrograms per unit dose.

Embodiment 145. The combination according to Embodiment 141, wherein the therapeutic amount of latrepirdine or a pharmaceutically acceptable salt thereof includes from about 10 mg to about 100 mg per unit dose.

Embodiment 146. The combination according to Embodiment 141, wherein dexmedetomidine and latrepirdine or pharmaceutically acceptable salts thereof are administered sublingually or buccally in a dosage form selected from the group consisting of a film, wafer, patch, lozenge, gel, spray, tablet and liquid drops.

Embodiment 147. The combination according to Embodiment 146, wherein dexmedetomidine and latrepirdine or pharmaceutically acceptable salts thereof are administered sublingually in the form of a tablet.

Embodiment 148. The combination according to Embodiment 146, wherein dexmedetomidine and latrepirdine or pharmaceutically acceptable salts thereof are administered buccally in the form of a tablet.

Embodiment 149. The combination according to Embodiments 147 or 148, wherein the tablet disintegrates in not less than 1 minute upon contact with an oral mucosa.

Embodiment 150. The combination according to Embodiment 141, wherein dexmedetomidine and latrepirdine or pharmaceutically acceptable salts thereof are administered simultaneously in a single dosage form.

Embodiment 151. The combination according to Embodiment 141, wherein dexmedetomidine and latrepirdine or pharmaceutically acceptable salts thereof are administered concurrently or sequentially in separate dosage forms.

Embodiment 152. The combination according to Embodiment 141, wherein dexmedetomidine and latrepirdine or pharmaceutically acceptable salts thereof are administered once daily, twice daily, thrice daily, four times daily, five times daily, or six times daily.

Embodiment 153. A kit of parts comprising at least two separate oromucosal lyophilized tablet dosage forms (a) and (b), the said dosage forms comprise:
(a) a therapeutically effective amount of dexmedetomidine or a pharmaceutically acceptable salt thereof;
(b) a therapeutically effective amount of latrepirdine or a pharmaceutically acceptable salt thereof; and optionally
(c) instructions for the simultaneous, sequential or separate administration of (a) and (b) to a subject in need thereof.

Embodiment 154. A kit of parts comprising a single oromucosal lyophilized tablet dosage form comprising:
(i) a therapeutically effective amount of dexmedetomidine or a pharmaceutically acceptable salt thereof;
(ii) a therapeutically effective amount of latrepirdine or a pharmaceutically acceptable salt thereof; and optionally
(iii) instructions for the administration of the single dosage form to a subject in need thereof.

Embodiment 155. The kit according to Embodiment 153 or 154, wherein dexmedetomidine or a pharmaceutically acceptable salt thereof is dexmedetomidine hydrochloride.

Embodiment 156. The kit according to Embodiment 153 or 154, wherein latrepirdine or a pharmaceutically acceptable salt thereof is latrepirdine hydrochloride.

Embodiment 157. The kit according to Embodiment 153 or 154, wherein latrepirdine or a pharmaceutically acceptable salt thereof is present in an amount of about 10 mg to about 100 mg.

Embodiment 158. The kit according to Embodiment 154, wherein dexmedetomidine and latrepirdine or pharmaceutically acceptable salts thereof are administered once daily, twice daily, thrice daily, four times daily, five times or six times daily in a single unit dose.

Embodiment 159. The kit according to any one of Embodiments 153 to 158, wherein dexmedetomidine and latrepirdine or pharmaceutically acceptable salts thereof are administered together or separately as a sublingual or buccal tablet.

Embodiment 160. The kit according to Embodiment 159, wherein the tablet disintegrates in not less than about 1 minute upon contact with an oral mucosa.

Embodiment 161. The kit according to any one of Embodiments 153 to 160, wherein dexmedetomidine and latrepirdine or pharmaceutically acceptable salts thereof are administered for the treatment of agitation caused by noradrenergic hyperarousal.

Embodiment 162. The kit according to Embodiment 161, wherein the agitation is acute or chronic.

Embodiment 163. The kit according to Embodiment 161, wherein the agitation caused by noradrenergic hyperarousal is associated with neurodegenerative disorders selected from a group consisting of dementia, Alzheimer's disease, frontotemporal dementia, Parkinsonism, agitation associated with sundown syndrome in Alzheimer's disease/dementia, or agitation/symptoms of agitation associated with other neurodegenerative disorders.

Embodiment 164. The kit according to Embodiment 161, wherein said agitation caused by noradrenergic hyperarousal is associated with neuropsychiatric disorders selected from a group consisting of schizophrenia, bipolar disorder, bipolar mania, delirium, depression, or another related neuropsychiatric disorder.

Embodiment 165 The kit according to embodiment 161, wherein said agitation caused by noradrenergic hyperarousal is associated with alcohol withdrawal, opioid use disorder, opioid withdrawal, substance abuse withdrawal or OPD/IPD procedure (e.g. MRI, CT or CAT scan, lumbar puncture, bone marrow aspiration/biopsy, tooth extraction or other dental procedures).

Embodiment 166. The kit according to Embodiment 155, wherein dexmedetomidine or a pharmaceutically acceptable salt thereof is present in an amount of about 10 micrograms to 300 micrograms (including about 30 micrograms, about 60 micrograms, about 90 micrograms, about 120 micrograms, about 180 micrograms, about 240 micrograms).

Embodiment 167. The method/dosage form/combination or kit according to any of the preceding embodiments, wherein the agitation is treated in the subject without causing significant sedation.

Embodiment 168. The method/dosage form/combination or kit according to any of the preceding embodiments, wherein the agitation is treated in the subject without causing cardiovascular effects.

Embodiment 169. A method of treating psychosis in a subject in need thereof, comprising administering a therapeutically effective amount of latrepirdine or a pharmaceutically acceptable salt thereof to the subject.

Embodiment 170. The method of treatment according to embodiment 169, wherein the latrepirdine is administered in a dosage amount of about 1 mg to about 100 mg once a day, about 10 mg once a day, about 20 mg once a day, about 30 mg once a day, about 20 mg twice a day. about 20 mg thrice a day, about 30 mg once a day, about 30 mg twice a day, about 60 mg once a day.

Embodiment 171. A method of treating psychosis in a subject in need thereof, comprising administering oromucosally a therapeutically effective amount of latrepirdine or a pharmaceutically acceptable salt thereof to the subject.

Embodiment 172. A method of treating psychosis in a subject in need thereof, the method comprising administering to the subject a therapeutically effective amount of dexmedetomidine or a pharmaceutically acceptable salt thereof in combination with a therapeutically effective amount of latrepirdine or a pharmaceutically acceptable salt thereof.

Embodiment 173. A method of treating psychosis in a subject in need thereof, the method comprising administering oromucosally to the subject a therapeutically effective amount of dexmedetomidine or a pharmaceutically acceptable salt thereof in combination with a therapeutically effective amount of latrepirdine or a pharmaceutically acceptable salt thereof.

Embodiment 174. A method of treating depression in a subject in need thereof, the method comprising administering oromucosally to the subject about 1 mg to about 100 mg of latrepirdine or a pharmaceutically acceptable salt thereof and about 5 micrograms to about 300 micrograms of dexmedetomidine or a pharmaceutically acceptable salt thereof.

Embodiment 175. The method according to embodiments 169 to 174, wherein the treatment is effective without causing significant sedation.

Embodiment 176. The method according to embodiments 169 to 174, wherein, the treatment is effective without experiencing clinically significant cardiovascular effects.

Embodiment 177. The method according to embodiments 169 to 174, wherein the severity of psychosis in the subject is assessed using PANS S scale.

Embodiment 178. The method according to embodiments 169 to 174, wherein the psychosis is acute.

Embodiment 179. The method according to embodiments 169 to 174, wherein the psychosis is chronic.

Embodiment 180. The method according to embodiments 169 to 174, wherein the subject is agitated.

Embodiment 181. The method according to embodiments 169 to 174, wherein the subject is non-agitated.

Embodiment 182. A method of achieving a PANS S score reduction in psychosis for a sustained period of time in a subject comprising oromucosally administering to the subject a therapeutically effective amount of latrepirdine or a pharmaceutically acceptable salt thereof.

Embodiment 183. A method of achieving a PANSS score reduction in psychosis for a sustained period of time in a subject comprising administering oromucosally to the subject a therapeutically effective amount of dexmedetomidine or a pharmaceutically acceptable salt thereof in combination with a therapeutically effective amount of latrepirdine or a pharmaceutically acceptable salt thereof.

Embodiment 184. The method according to embodiments 182 or 183, wherein the PANSS score reduction is at least about 20% to about 50% from baseline score.

Embodiment 185. The method according to embodiment 184, wherein the PANS S score reduction is about 25% from baseline score.

Embodiment 186. The method according to embodiment 184, wherein the PANS S score reduction is about 50% points from baseline score

Embodiment 187. The method according to any of embodiments 169-186, wherein the psychosis is associated with a neuropsychiatric disorder selected from the group consisting of schizophrenia, schizoaffective disorder, depression, dementia and bipolar disorder or another related neuropsychiatric disorder.

Embodiment 188. The method according to any of embodiments 169-186, wherein psychosis is associated with neurodegenerative disorders.

Embodiment 189. The method according to any of embodiments 169-186, wherein the psychosis is associated with diseased condition such as substance abuse disorders (e.g., alcohol, opioid and other substance withdrawal).

Embodiment 190. The method according to any of embodiments 169-189, wherein the psychosis is a single, recurrent or mixed episode.

The details of the disclosure, its objects and advantages are explained hereunder in greater detail in relation to non-limiting exemplary illustrations.

### EXAMPLES

### Example 1. Latrepirdine Dihydrochloride Hydrate and Dexmedetomidine Hydrochloride Sublingual/Buccal/Gingival Tablets with Mucoadhesive Properties

TABLE 1
Latrepirdine oromucosal tablets (A to F)

| | Example | | Example | | Example | | Example | | Example | | Example | |
|---|---|---|---|---|---|---|---|---|---|---|---|---|
| Ingredients | A | | B | | C | | D | | E | | F | |
| Latrepirdine | 30 | mg | 30 | mg | 30 | mg | 30 | mg | 30 | mg | 30 | mg |
| Hypromellose (lower | 3.0 | mg | | | | | | | | | | |
| viscosity grades like K100L | | | | | | | | | | | | |
| or 4000 cps or less) | | | | | | | | | | | | |
| Hydroxy propyl cellulose | | | 2.5 | mg | | | | | | | | |
| (Lower viscosity grades of | | | | | | | | | | | | |
| MW <150K daltons) | | | | | | | | | | | | |
| Polyethylene oxide | | | | | 2.5 | mg | | | | | | |
| Xanthan gum (or) Sodium | | | | | | | 2.5 | mg | | | | |
| alginate | | | | | | | | | | | | |
| Carbomer (lower viscosity | | | | | | | | | 2.0 | mg | | |
| grades) | | | | | | | | | | | | |
| Croscarmellose Sodium (or) | 2.0 | mg | 2.0 | mg | 2.0 | mg | 2.0 | mg | 2.0 | mg | 2.0 | mg |
| sodium starch glycolate | | | | | | | | | | | | |
| Sucralose | 0.5 | mg | 0.5 | mg | 0.5 | mg | 0.5 | mg | 0.5 | mg | 0.5 | mg |
| Magnesium Stearate (and)/ | 0.5 | mg | 0.5 | mg | 0.5 | mg | 0.5 | mg | 0.5 | mg | 0.5 | mg |
| (or) Silicon dioxide | | | | | | | | | | | | |
| Lactose Monohydrate (or) | 24.4 | mg | 24.9 | mg | 24.9 | mg | 24.9 | mg | 25.4 | mg | 25.4 | mg |
| Mannitol | | | | | | | | | | | | |
| Tablet weight | 60.0 | mg | 60.0 | mg | 60.0 | mg | 60.0 | mg | 60.0 | mg | 60.0 | mg |

TABLE 2
Latrepirdine oromucosal tablets (G to L)

| | Example | | Example | | Example | | Example | | Example | | Example | |
|---|---|---|---|---|---|---|---|---|---|---|---|---|
| Ingredients | G | | H | | I | | J | | K | | L | |
| Latrepirdine | 30 | mg | 30 | mg | 30 | mg | 30 | mg | 30 | mg | 30 | mg |
| Hypromellose (lower | 3.0 | mg | | | | | | | | | | |
| viscosity grades like | | | | | | | | | | | | |
| K100L or 4000 cps or | | | | | | | | | | | | |
| less) | | | | | | | | | | | | |
| Hydroxy propyl | | | 2.5 | mg | | | | | | | | |
| cellulose (Lower | | | | | | | | | | | | |
| viscosity grades of | | | | | | | | | | | | |
| MW <150K daltons) | | | | | | | | | | | | |
| Polyethylene oxide | | | | | 2.5 | mg | | | | | | |
| Xanthan gum (or) | | | | | | | 2.5 | mg | | | | |
| Sodium alginate | | | | | | | | | | | | |
| Carbomer (lower | | | | | | | | | 2.0 | mg | | |
| viscosity grades) | | | | | | | | | | | | |
| Gelatin (low | 1.5 | mg | 1.5 | mg | 1.5 | mg | 1.5 | mg | 1.5 | mg | 1.5 | mg |
| viscosity) | | | | | | | | | | | | |
| Croscarmellose | 2.0 | mg | 2.0 | mg | 2.0 | mg | 2.0 | mg | 2.0 | mg | 2.0 | mg |
| Sodium (or) sodium | | | | | | | | | | | | |
| starch glycollate | | | | | | | | | | | | |
| Sucralose | 0.5 | mg | 0.5 | mg | 0.5 | mg | 0.5 | mg | 0.5 | mg | 0.5 | mg |
| Magnesium Stearate | 0.5 | mg | 0.5 | mg | 0.5 | mg | 0.5 | mg | 0.5 | mg | 0.5 | mg |
| (and)/(or) Silicon | | | | | | | | | | | | |
| dioxide | | | | | | | | | | | | |
| Lactose Monohydrate | 24.4 | mg | 24.9 | mg | 24.9 | mg | 24.9 | mg | 25.4 | mg | 25.4 | mg |
| (or) Mannitol | | | | | | | | | | | | |
| Tablet weight | 60.0 | mg | 60.0 | mg | 60.0 | mg | 60.0 | mg | 60.0 | mg | 60.0 | mg |

TABLE 3
Dexmedetomidine and latrepirdine oromucosal tablets (M to R)

| | Example | | Example | | Example | | Example | | Example | | Example | |
|---|---|---|---|---|---|---|---|---|---|---|---|---|
| Ingredients | M | | N | | O | | P | | Q | | R | |
| Dexmedetomidine | 120 | mcg | 120 | mcg | 120 | mcg | 120 | mcg | 120 | mcg | | |
| Latrepirdine | 30 | mg | 30 | mg | 30 | mg | 30 | mg | 30 | mg | | |
| Hypromellose (lower | 3.0 | mg | | | | | | | | | | |
| viscosity grades like | | | | | | | | | | | | |
| K100L or 4000 cps or | | | | | | | | | | | | |
| less) | | | | | | | | | | | | |
| Hydroxy propyl | | | 2.5 | mg | | | | | | | | |
| cellulose (Lower | | | | | | | | | | | | |
| viscosity grades of | | | | | | | | | | | | |
| MW <150K daltons) | | | | | | | | | | | | |
| Polyethylene oxide | | | | | 2.5 | mg | | | | | | |
| Xanthan gum (or) | | | | | | | 2.5 | mg | | | | |
| Sodium alginate | | | | | | | | | | | | |
| Carbomer (lower | | | | | | | | | 2.0 | mg | | |
| viscosity grades) | | | | | | | | | | | | |
| Croscarmellose | 2.0 | mg | 2.0 | mg | 2.0 | mg | 2.0 | mg | 2.0 | mg | 2.0 | mg |
| Sodium (or) sodium | | | | | | | | | | | | |
| starch glycollate | | | | | | | | | | | | |
| Sucralose | 0.5 | mg | 0.5 | mg | 0.5 | mg | 0.5 | mg | 0.5 | mg | 0.5 | mg |
| Magnesium Stearate | 0.5 | mg | 0.5 | mg | 0.5 | mg | 0.5 | mg | 0.5 | mg | 0.5 | mg |
| (and)/(or) Silicon | | | | | | | | | | | | |
| dioxide | | | | | | | | | | | | |
| Lactose Monohydrate | 24.4 | mg | 24.9 | mg | 24.9 | mg | 24.9 | mg | 25.4 | mg | 25.4 | mg |
| (or) Mannitol | | | | | | | | | | | | |
| Tablet weight | 60.0 | mg | 60.0 | mg | 60.0 | mg | 60.0 | mg | 60.0 | mg | 60.0 | mg |

TABLE 4
Dexmedetomidine and latrepirdine oromucosal tablets (S to X)

| | Example | | Example | | Example | | Example | | Example | | Example | |
|---|---|---|---|---|---|---|---|---|---|---|---|---|
| Ingredients | S | | T | | U | | V | | W | | X | |
| Dexmedetomidine | 120 | mcg | 120 | mcg | 120 | mcg | 120 | mcg | 120 | mcg | | |
| Latrepirdine | 30 | mg | 30 | mg | 30 | mg | 30 | mg | 30 | mg | | |
| Hypromellose (lower | 3.0 | mg | | | | | | | | | | |
| viscosity grades like | | | | | | | | | | | | |
| K100L or 4000 cps or | | | | | | | | | | | | |
| less) | | | | | | | | | | | | |
| Hydroxy propyl | | | 2.5 | mg | | | | | | | | |
| cellulose (Lower | | | | | | | | | | | | |
| viscosity grades of | | | | | | | | | | | | |
| MW <150K daltons) | | | | | | | | | | | | |
| Polyethylene oxide | | | | | 2.5 | mg | | | | | | |
| Xanthan gum (or) | | | | | | | 2.5 | mg | | | | |
| Sodium alginate | | | | | | | | | | | | |
| Carbomer (lower | | | | | | | | | 2.0 | mg | | |
| viscosity grades) | | | | | | | | | | | | |
| Gelatin (low viscosity) | 1.5 | mg | 1.5 | mg | 1.5 | mg | 1.5 | mg | 1.5 | mg | 1.5 | mg |
| Croscarmellose | 2.0 | mg | 2.0 | mg | 2.0 | mg | 2.0 | mg | 2.0 | mg | 2.0 | mg |
| Sodium (or) sodium | | | | | | | | | | | | |
| starch glycollate | | | | | | | | | | | | |
| Sucralose | 0.5 | mg | 0.5 | mg | 0.5 | mg | 0.5 | mg | 0.5 | mg | 0.5 | mg |
| Magnesium Stearate | 0.5 | mg | 0.5 | mg | 0.5 | mg | 0.5 | mg | 0.5 | mg | 0.5 | mg |
| (and)/(or) Silicon | | | | | | | | | | | | |
| dioxide | | | | | | | | | | | | |
| Lactose Monohydrate | 24.4 | mg | 24.9 | mg | 24.9 | mg | 24.9 | mg | 25.4 | mg | 25.4 | mg |
| (or) Mannitol | | | | | | | | | | | | |
| Tablet weight | 60.0 | mg | 60.0 | mg | 60.0 | mg | 60.0 | mg | 60.0 | mg | 60.0 | mg |

The tablets described in this example (see Tables 1 to 4) may be made in accordance with the process described herein. A representative process is illustrated below.
1. Disperse the drug substance (latrepirdine and/or dexmedetomidine), a binder, sweetener in water.
2. Prepare and sift a blend of rest of the ingredients except magnesium stearate/silicon dioxide.
3. Granulate the blend by using the solution of step 1 in a suitable rapid mixer granulator.
4. Dry the granules in a suitable fluid bed drier.
5. Size the dried granules appropriately in quadro-co-mill or multimill and load into a suitable blender such as V-blender.
6. Lubricate with magnesium stearate/silicon dioxide and compress the final lubricated blend into tablets of specific dimensions using appropriate tooling.

### Example 2. The Effect of Latrepirdine Dihydrochloride Hydrate in Yohimbine-Induced Noradrenergic Mediated Hyperarousal in Wistar Rats

Background: Yohimbine is an alpha 2-adrenergic receptor antagonist reported to trigger panic attacks in healthy volunteers and to exacerbate symptoms in patients with panic disorder (Charney et al., 1992 Acta Psychiatr Scand., Vol. 86(4): 273-282). Peripheral administration of Yohimbine causes noradrenergic mediated hyperarousal in rats as assessed by the EPM paradigm.

Rationale: The drug yohimbine is a natural product used to test a specific pathway, noradrenergic-mediated hyper-arousal. It is well established that yohimbine is an alpha2-adrenergic receptor antagonist and activates the locus coeruleus (LC) and increases noradrenergic signaling (the opposite of alpha2-adrenergic agonists like dexmedetomidine and clonidine). Increased LC activity and noradrenergic signaling makes animals anxious and agitated which can be measured by using the elevated plus maze. In the elevated plus maze, rodents were in the closed arms and explored the open arms depending on their anxiety and agitation (rats and mice normally explore their environments extensively when calm). By administering yohimbine, animals generally stayed in the closed arms and did not explore open arms.

Animal groups: Male: Wistar rats were used in this study and were randomly divided to different experimental groups (12 animals per group).

Acclimatization: Animals were numbered and kept in acclimatization for a period of 5-7 days before the initiation of the experiment. During the period of acclimation, male Wistar rats were examined on a regular basis, handled, and weighed to assure adequate health and suitability.

Diet and Water: Animals were maintained on the normal rodent chow ad libitum and given free access to fresh autoclaved potable drinking water.

All the experiments on animals were conducted in accordance with the guidelines of the Committee for the Purpose of Control and Supervision of Experiments on Animals (CPCSEA), Government of India the Association for Assessment and Accreditation of Laboratory Animal Care international (AAALAC).

Study drugs: Four test drugs with different mechanisms of action linked to agitation were chosen to find drugs that are effective in reducing yohimbine-induced agitation: (1) Fluvoxamine (selective serotonin reuptake inhibitor) (2) Dextromethorphan (modulator of NMDA signals) (3) Diazepam (modulator of gamma-aminobutyric acid (GABA) as a positive control) and (4) Latrepirdine (complex pharmacology).

TABLE 5
Treatment group details:

| Group | | | n per |
|---|---|---|---|
| No. | Treatment* | Dose and route | group |
| 1 | Vehicle 1 | Saline; i.m. | 12 |
| 2 | Vehicle 2 | Saline + 30% Ethanol; i.m. | 12 |
| 3 | Latrepirdine | 3 mg/kg; i.m. | 12 |
| | dihydrochloride hydrate | | |
| 4 | Latrepirdine | 10 mg/kg; i.m. | 12 |
| | dihydrochloride hydrate | | |
| 5 | Dextromethorphan | 10 mg/kg; i.m. | 12 |
| | hydrobromide | | |
| 6 | Fluvoxamine maleate | 10 mg/kg; i.m. | 12 |
| 7 | Diazepam | 1 mg/kg; i.p. | 12 |

*weight was calculated based on free base.
IM = Intramuscular; IP: Intraperitoneal.
All treatment groups were pre-treated with Yohimbine 2.5 mg/kg

The test drugs were procured from Sigma or Tocris. dextromethorphan from Sigma Aldrich, Cat No. D9684; latrepirdine from Sigma Aldrich, Cat No. D6196 or Tocris Cat No. 3201 diazepam and fluvoxamine Sigma Aldrich, Cat No. F2802 or Tocris, Cat No. 1033. All these test drugs were formulated in sufficient quantity in 0.9% saline except for Fluvoxamine, which was formulated in 30% ethanol in 0.9% saline.

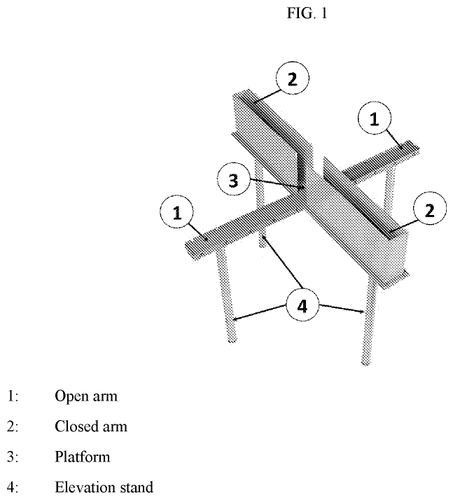

Apparatus: Elevated Plus Maze (EPM) was used (FIG. 1). The maze consisted of two closed arms and two open arms forming a cross, with a square center platform. All visible surfaces were made of black acrylic. Each arm of the maze was placed on a support column above the floor. Antistatic black vinyl curtains used for surrounding the EPM to make an enclosure.

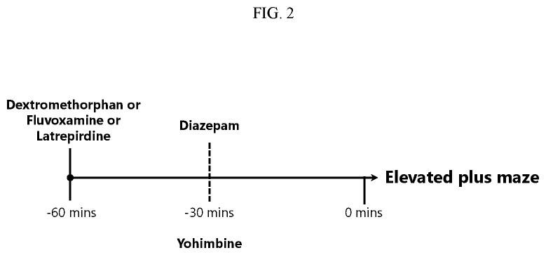

Study Design and Dosing: Yohimbine was administered by intraperitoneal injection (i.p.) at a dose of 2.5 mg/kg (30 minutes prior to study) to induce agitation and the effects were assessed 30 minutes after yohimbine injection by measuring the number of open arm entries and exploration time in the EPM. The test drugs fluvoxamine, dextromethorphan and latrepirdine dihydrochloride hydrate were administered through intramuscular route and, diazepam through intraperitoneal route into the hindleg thigh muscle of rats (60 minutes prior to study; 30 minutes prior to yohimbine administration). Diazepam was injected 30 minutes intraperitonially prior to the assay i.e. along with yohimbine administration. The study design is illustrated in FIG. 2 and table 5.

After the test drug administration, the rat was placed on the platform opposite a closed arm. The number of entries and the time spent in each arm were recorded during a 5 min period. The animal was considered as entered in arm when it placed its four paws into the arm.

Statistical Analysis: Analysis of variance (ANOVA) was performed on the result data. Fisher's PLSD was used for pairwise comparisons and p value ≤0.05 was considered significant.

Results: Unexpectedly, it was found that noradrenergic mediated hyperarousal was reduced by pre-treating animals for 1 hour with latrepirdine dihydrochloride hydrate (3 mg/kg and 10 mg/kg, i.m.), as indicated by a significantly increased number of open arm entries and increased exploration time. In other words, latrepirdine dihydrochloride hydrate reversed yohimbine-induced noradrenergic mediated hyperarousal. The effect of latrepirdine dihydrochloride hydrate on reduction of noradrenergic mediated hyperarousal was specific since pretreatment of animals with dextromethorphan (at 10 mg/kg, i.m.) or fluvoxamine (at 10 mg/kg, i.m.) was ineffective. Pretreatment with diazepam (at 1 mg/kg, i.p.) served as a positive control.

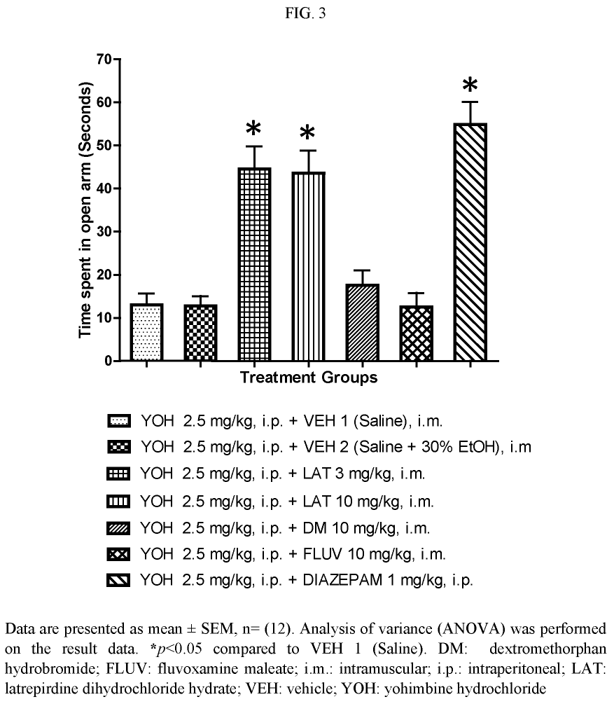

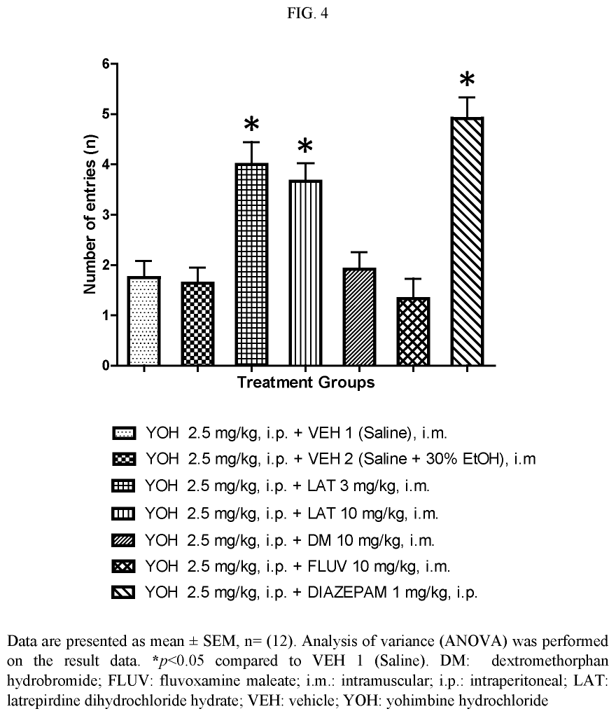

FIGS. 3 and 4 showed that latrepirdine dihydrochloride hydrate significantly increased the number of entries into the open arms and the time spent (seconds) in the open arms, comparable to diazepam (at 1 mg/kg, i.p.). Other test drugs dextromethorphan (at 10 mg/kg, i.m) and fluvoxamine (at 10 mg/kg, i.m) showed no effect.

Conclusions: Latrepirdine dihydrochloride hydrate was able to reduce behaviors associated with high noradrenergic signaling and its pretreatment reverses anxious behavior in rats that was caused by selective alpha 2-adrenergic receptor antagonism, providing support for a putative poly pharmacology approach in hyperarousal related disorders with compounds acting via alpha-2 adrenergic receptor agonism. Therefore, latrepirdine dihydrochloride hydrate should be effective in those patients with associated high noradrenergic mediated hyper-arousal.

### Example 3. To Evaluate the Effect of Latrepirdine Dihydrochloride Hydrate at Different Doses in Yohimbine-Induced Noradrenergic Hyperarousal in Wistar Rats

Materials and Methods:

Drug preparation: All compounds/drugs were prepared in normal saline (0.9% NaCl).

Yohimbine was prepared at a solution of 2.5 mg/mL which when injected i.p. at a dosage volume of 1 mL/kg results in a dose of 2.5 mg/kg. Diazepam was prepared at a solution of 1 mg/mL which when injected i.p. at a dosage volume of 1 mL/kg results in a dose of 1 mg/kg. Latrepirdine dihydrochloride hydrate was prepared at a solution of 7.5, 2.5, 0.75 and 0.25 mg/mL which when injected i.m. at a volume of 100 μL for a rat of 250 g results in doses of 3; 1; 0.3 and 0.1 mg/kg; respectively. Latrepirdine dihydrochloride hydrate was also prepared at 0.3 mg/mL which when administrated i.p. at a dosage volume of 1 mL/kg results in a dose of 3 mg/kg.

Test Animals:

Male Wistar rats (Janvier; Le Genest St Isle—France) were used for the study. They were purchased at a body weight of 150 g and reached a body weight of about 220 g at the time of use. They were group-housed (3-4 rats per cage) and maintained in a room with controlled temperature (21-22° C.) and a reversed light-dark cycle (12 h/12 h; lights on: 17:30-05:30; lights off: 05:30-17:30) with food and water available ad libitum.

Treatment Schedule:

Rats were distributed in 9 different experimental groups with 12 rats per group as illustrated in Table 6. Each experimental group was also coded so that the experimenter became unaware of the true experimental group at the time EPM trial. The dose, the route and the pre-treatment time are also provided in the Table 6.

TABLE 6
Treatment schedule

| Group | | |
|---|---|---|
| Number | n | Names/Descriptions |
| 1 | 12 | Saline (i.p., 30 min)/Vehicle (i.m., 60 min) |
| 2 | 12 | Yohimbine hydrochloride (2.5 mg/kg/mL; i.p., 30 min.)/ |
| | | Vehicle (i.m., 60 min) |
| 3 | 12 | Yohimbine hydrochloride (2.5 mg/kg/mL; i.p., 30 min.)/ |
| | | Diazepam (1 mg/kg, i.p., 30 min) |
| 4 | 12 | Saline (i.p., 30 min)/Latrepirdine dihydrochloride |
| | | hydrate (3 mg/kg; i.m., 60 min) |
| 5 | 12 | Yohimbine hydrochloride (2.5 mg/kg/mL; i.p., 30 min.)/ |
| | | Latrepirdine dihydrochloride hydrate (3 mg/kg; i.p., |
| | | 60 min) |
| 6 | 12 | Yohimbine hydrochloride (2.5 mg/kg/mL; i.p., 30 min.)/ |
| | | Latrepirdine dihydrochloride hydrate (0.1 mg/kg; i.m., |
| | | 60 min) |
| 7 | 12 | Yohimbine hydrochloride (2.5 mg/kg/mL; i.p., 30 min.)/ |
| | | Latrepirdine dihydrochloride hydrate (0.3 mg/kg; i.m., |
| | | 60 min) |
| 8 | 12 | Yohimbine hydrochloride (2.5 mg/kg/mL; i.p., 30 min.)/ |
| | | Latrepirdine dihydrochloride hydrate (1 mg/kg; i.m., |
| | | 60 min) |
| 9 | 12 | Yohimbine hydrochloride (2.5 mg/kg/mL; i.p., 30 min.)/ |
| | | Latrepirdine dihydrochloride hydrate (3 mg/kg; i.m., |
| | | 60 min) |

* weight was calculated based on free base.

Experimental Procedure:

The rats were randomly assigned to one of the different experimental groups. Each animal was identified by its group name, cage number, series (day) of experiment, and a number (from 1 to 9) written with permanent ink on its tail. They were handled by an experimenter for about 3 min each day during 1 week prior to the day of EPM trial. The apparatus was a PVC maze covered with Plexiglas and subdivided into four equal exploratory arms (40×10 cm), which were all interconnected by a small platform (10×10 cm). The apparatus was placed 65 cm above the floor. Two arms were opened, and two others were closed with wall (high: 10 cm).

After compound administration, rat was placed on the platform opposite a closed arm. The number of entries and the time spent in each arm were recorded during a 5 min period. The animal was considered as entered in an arm when it placed its four paws in the arm. The apparatus was cleaned between each animal using alcohol (70%). Urine and feces were removed from the maze. During the trials, animal handling and the visibility of the operator were minimized as much as possible.

Calculation and Statistical Analysis:

Analysis of variance (ANOVA) was performed on the result data. Fisher's PLSD was used for pairwise comparisons and p value ≤0.05 were considered significant. For the sake of comparison, the change in the EPM performance of Yohimbine rats was expressed as a percentage of increase/decrease as referred to the level recorded in the vehicle (set as 0% change). Therefore, for each of the EPM parameter, the following formula was used: [performance of a given group−performance of vehicle group]/[performance of vehicle group]×100

Results

General health: No sign of macroscopically visible side effect related to the compound treatment was observed.

Behavior in the EPM

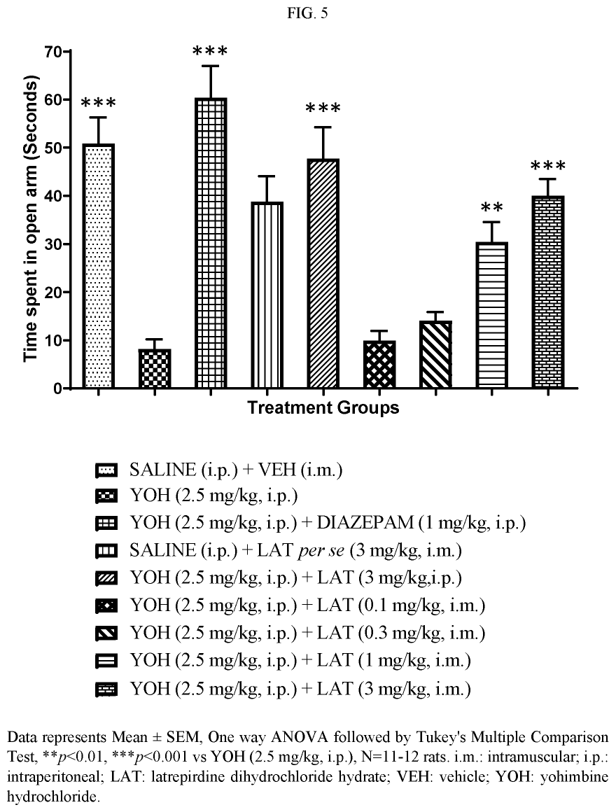

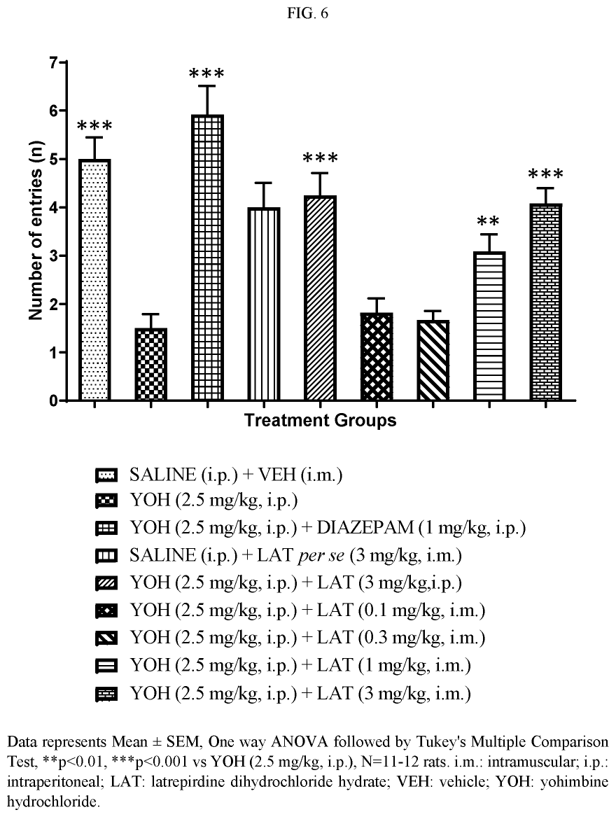

FIGS. 5 and 6 showed the average performance of each experimental group in the term of number of entries to and the time spent into the open arms, respectively. Table 7 provides the percentage of change (decrease or increase) in the EPM parameters as referred to the vehicle performance level (set as 0% change). As shown in FIGS. 5 and 6, vehicle-treated yohimbine administered rats showed a marked significant decrease in the number of visits to and in the time spent into the open arms as compared to vehicle-treated saline rats. This suggests the occurrence of yohimbine-induced noradrenergic mediated hyperarousal as assessed in the EPM paradigm. Yohimbine-induced noradrenergic mediated hyperarousal was reversed by 1 mg/kg diazepam treatment as shown by the significant increase in the in the number of visits to and in the time spent into the open arms (294% and 642% increase respectively) (Table 7). Further latrepirdine dihydrochloride hydrate induced a dose-dependent increase in the number of visits to and in the time spent into the open arms of yohimbine administered rats and effect was significant at the doses of 1 and 3 mg/kg as shown in table 7 and FIGS. 5 and 6. It was also observed that the EPM performance of yohimbine rats treated with latrepirdine dihydrochloride hydrate (3 mg/kg i.m) was comparable to that of specimens treated intraperitoneally, suggesting that intramuscular (i.m.) latrepirdine dihydrochloride hydrate is as effective as that administrated by intraperitoneal (i.p.) route (see Table 7 and FIGS. 5 and 6). It is noteworthy that Latrepirdine dihydrochloride hydrate (3 mg/kg i.m) did not significantly modify the EPM performance of saline-treated rats (yohimbine-free rats), suggesting that latrepirdine dihydrochloride hydrate did not induce any significant anti-agitation behavior in yohimbine-free rats (see Table 7 and FIGS. 5 and 6).

TABLE 7
Change in the behavioral performance during the EPM test

| | Percent increase as referred to | |
|---|---|---|
| | the Vehicle level | |
| Name/Descriptions | Entries | Time |
| Saline (i.p., 30 min)/Vehicle (i.m., 60 min) | 233 | 525 |
| Yohimbine hydrochloride (2.5 mg/kg/mL; i.p., 30 min.)/ | 0 | 0 |
| Vehicle (i.m., 60 min) | | |
| Yohimbine hydrochloride (2.5 mg/kg/mL; i.p., 30 min.)/ | 294 | 642 |
| Diazepam (1 mg/kg, i.p., 30 min) | | |
| Saline (i.p., 30 min)/Latrepirdine dihydrochloride hydrate | 167 | 377 |
| (3 mg/kg; i.m., 60 min) | | |
| Yohimbine hydrochloride (2.5 mg/kg/mL; i.p., 30 min.)/ | 183 | 486 |
| Latrepirdine dihydrochloride hydrate (3 mg/kg; i.p., 60 min) | | |
| Yohimbine hydrochloride (2.5 mg/kg/mL; i.p., 30 min.)/ | 21 | 21 |
| Latrepirdine dihydrochloride hydrate (0.1 mg/kg; i.m., 60 min) | | |
| Yohimbine hydrochloride (2.5 mg/kg/mL; i.p., 30 min.)/ | 11 | 73 |
| Latrepirdine dihydrochloride hydrate (0.3 mg/kg; i.m., 60 min) | | |
| Yohimbine hydrochloride (2.5 mg/kg/mL; i.p., 30 min.)/ | 106 | 274 |
| Latrepirdine dihydrochloride hydrate (1 mg/kg; i.m., 60 min) | | |
| Yohimbine hydrochloride (2.5 mg/kg/mL; i.p., 30 min.)/ | 172 | 392 |
| Latrepirdine dihydrochloride hydrate (3 mg/kg; i.m., 60 min) | | |

CONCLUSION: The results showed that latrepirdine dihydrochloride hydrate (at 1 and 3 mg/kg) significantly reduced noradrenergic mediated hyperarousal in yohimbine administered rats in dose-dependent manner (see table 7 and FIGS. 5 and 6).

### Example 4. To Evaluate the Anti-Depressant Effect of Combination of Dexmedetomidine Hydrochloride and Latrepirdine Dihydrochloride Hydrate in Rat Forced Swim Test Model in Dark Phase

### Materials and Methods

Test System
Species: Rattus norvegicus
Strain: Sprague Dawley (SD)
Sex and Age: Male, 8-10 weeks old
Source: Hylasco Biotechnology Pvt. Ltd. 4B, M.N. Park, Turkaplly (Vil), Shameerpet (Mdl), Medchal Dist, -500078
No of groups: 7; Total number of animals: 78
Number of animals per group: 9-12

TABLE 8
Treatment group details:

| Group | | | n per | | | |
|---|---|---|---|---|---|---|
| No. | Treatment* | Dose and route | group | Dosing Volume | | Frequency |
| 1 | Vehicle | 0.9% saline; i.m. | 9 | 100 | μL/site | Single dose, 60 |
| | | | | | | min prior Swim 2 |
| 2 | Desipramine | 30 mg/kg in | 9 | 10 | mL/kg | Single dose, 60 |
| | hydrochloride | 0.9% saline; p.o | | | | min prior Swim 2 |
| 3 | Dexmedetomidine | 1 μg/kg in 0.9% | 12 | 100 | μL/site | Single dose, 60 |
| | hydrochloride | saline; i.m. | | | | min prior Swim 2 |
| 4 | Dexmedetomidine | 5 μg/kg in 0.9% | 12 | 100 | μL/site | Single dose, 60 |
| | hydrochloride | saline; i.m. | | | | min prior Swim 2 |
| 5 | Latrepirdine | 1 mg/kg in 0.9% | 12 | 100 | μL/site | Single dose, 60 |
| | dihydrochloride | saline; i.m. | | | | min prior Swim 2 |
| | hydrate | | | | | |

| | | | | | |
|---|---|---|---|---|---|
| 6 | Latrepirdine | 1 mg/kg in 0.9% | 12 | 50 μL/site | Single dose, 60 |
| | dihydrochloride | saline; i.m.; | | Latrepirdine + | min prior Swim 2 |
| | hydrate + | 1 μg/kg in 0.9% | | 50 μL/site | |
| | Dexmedetomidine | saline; i.m. | | Dexmedetomidine | |
| | hydrochloride | | | | |
| 7 | Latrepirdine | 1 mg/kg in 0.9% | 12 | 50 μL/site | Single dose, 60 |
| | dihydrochloride | saline; i.m.; | | Latrepirdine + | min prior Swim 2 |
| | hydrate + | 5 μg/kg in 0.9% | | 50 μL/site | |
| | Dexmedetomidine | saline; i.m. | | Dexmedetomidine | |
| | hydrochloride | | | | |

*weight was calculated based on free base.
i.m. = Intramuscular;
p.o. = Per Oral

TABLE 9
Treatment group staggering details

| | | | Co- | Co- | Co- |
|---|---|---|---|---|---|
| Group | | n per | hort | hort | hort |
| No. | Treatment | group | 1 (n) | 2 (n) | 3 (n) |
| 1 | Vehicle | 9 | 3 | 3 | 3 |
| 2 | Desipramine hydrochloride | 9 | 3 | 3 | 3 |
| 3 | Dexmedetomidine hydrochloride | 12 | 4 | 4 | 4 |
| 4 | Dexmedetomidine hydrochloride | 12 | 4 | 4 | 4 |
| 5 | Latrepirdine dihydrochloride | 12 | 4 | 4 | 4 |
| | hydrate | | | | |
| 6 | Latrepirdine dihydrochloride | 12 | 4 | 4 | 4 |
| | hydrate + Dexmedetomidine | | | | |
| | hydrochloride | | | | |
| 7 | Latrepirdine dihydrochloride | 12 | 4 | 4 | 4 |
| | hydrate + Dexmedetomidine | | | | |
| | hydrochloride | | | | |

### Environmental Conditions

Animals were housed in an environment-controlled room at 22±3° C. and relative humidity of 30 to 70 percent. During the study, 12/12 light/dark cycles were maintained. Lights of the acclimatization area were switched off at 06:00 PM. All experiments were carried out at ambient temperature under dark phase of lighting (between 06:00 PM to 06:00 AM). Adequate fresh air supply of 12-15 air changes/hour was maintained in the experimental room. The maximum and minimum temperature and relative humidity in the experimental rooms was recorded once daily. The relative humidity in the experimental room was calculated daily from dry and wet bulb temperature recordings.

### Housing

Animals were grouped housed 2 per cage in standard animal cages with facilities for pelleted food and drinking water in polycarbonate bottle with stainless steel sipper tubes. During the experimental period, animals were housed in a single experimental room.

Diet

Standard rodent diet was provided ad libitum to all animals.

Water

Bore-well water passed through activated charcoal filter and exposed to UV rays in Aquaguard water filter-cum-purifier manufactured by Eureka Forbes Ltd., Mumbai-400 001, India, were provided ad libitum to the animals.

### Randomization

Animals were distributed to different groups based on body weight data.

Protocol:

A. Acclimatization Phase:

Post quarantine period animals were acclimatized for 7 days. It was ensured that inter group body weight variations were minimal and does not exceed ±10% of the mean body weight across the groups.

Study was carried out as a staggered treatment, each treatment group divided as cohort 1, cohort 2 and cohort 3.

Swim 1 was carried out between 18:30 to 21:30.

Swim 2 was carried out between 19:30 to 21:30. Animals were administered test compounds from 18:30 onwards in staggered manner 60 minutes before FST 2.

Post intramuscular injection, animals were observed for any abnormal effect due to intramuscular injection.

Experimental room was equipped with 50-watt red light bulb during Swim 1 and Swim 2 procedure

B. Forced Swim Test

Forced Swim Test was carried out in Dark Phase

On Day 1, animals were subjected to forced swim test training for 15 minutes (Swim 1) and on Day 2, Swim 2 was carried out post treatment.

On day 2 animals were dosed with respective treatment 60 min prior to performing FST.

Forced swim test (Swim 2) was scored manually with no video recording

Forced swim test (FST), was used to study the depressive-like behavior in rodents. The test was carried out in transparent cylindrical glass containers measuring 46 cm in height and 20 cm in diameter.

The containers were filled with water (23-25° C.) to a depth of 30 cm.

Clean drying cages, heat lamps and heat pads were used for the animals that finished the procedure to avoid hypothermia.

All animals were handled for about 2 min daily for the 5 days prior to the beginning of the experimental procedure.

There were 2 swim sessions, 24 h apart. The first session, Swim 1 was the pretest/training stage (15 min) and the second session, Swim 2, was the test stage (8 min).

In swim 2, first 1-minute data was excluded from analysis and rest 7 minutes data was considered. In 7 minutes, data, it was divided 5 minutes and 2 minute.

Swim 1

Rat was placed in the water-filled cylinder for 15 min (Swim 1).

After 15 minutes have elapsed, rat was removed from the cylinder and placed in the transient drying cage with the heat lamp above it and the heat pad under it for 15 minutes.

After the pre-test Swim 1 and the 15-minute drying period, animals were returned to their home cages.

Water was changed after every session to avoid any influence on the next rat.

Swim 2

Twenty-four hours after the start of Swim 1, the test swim (Swim 2) was carried out.

Rat was placed in the water-filled cylinder for 8 min (Swim 2).

After 8 minutes elapsed, the rat from the container was taken and placed in the transient drying cage with the heat lamp above it and the heat pad under it for 15 minutes. The rat was closely and continuously monitored while recovering in this cage.

The water was changed after every session to avoid any influence on the next rat.

During Swim 2, animals were observed for floating with the absence of any movement (immobility), climbing, and swimming.

The duration of time spent immobile, swimming, and climbing was observed by an observer blind to the treatment group.

Observation Criteria

Immobility Period

A rat was judged to be immobile when it remained floating in the water without struggling and was making only those movements necessary to keep its head above water.

Swimming Behavior

A rat was judged to be swimming if it showed active horizontal (swimming) motions, more than necessary to merely maintain its head above water (e.g., moving around in the cylinder).

Climbing Behavior

A rat was judged to be climbing when it showed active vertical movements with its forepaws in and out of the water, usually directed against the walls.

Statistical Analysis

One Way ANOVA, Dunnet's multiple comparison test was applied with respect to vehicle saline and Tukey's multiple comparison test was applied to compare the data in between the groups using GraphPad Prism version 9 software.

Results

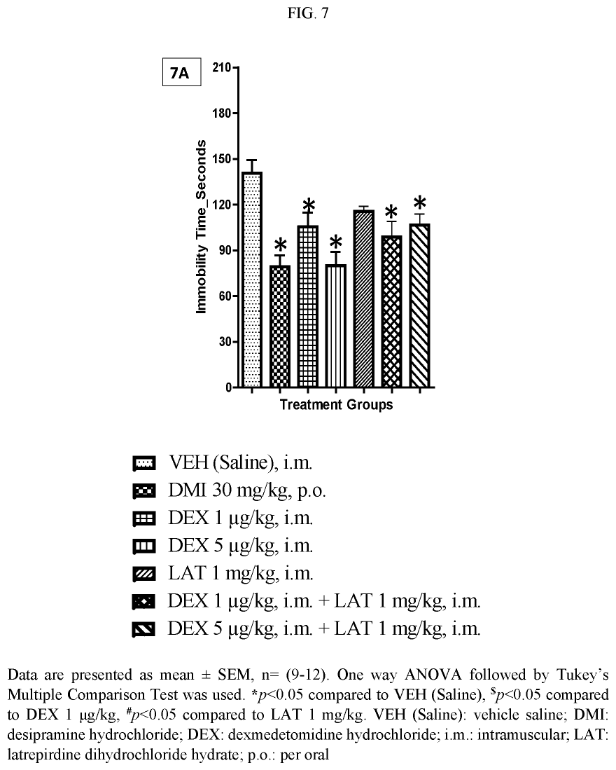

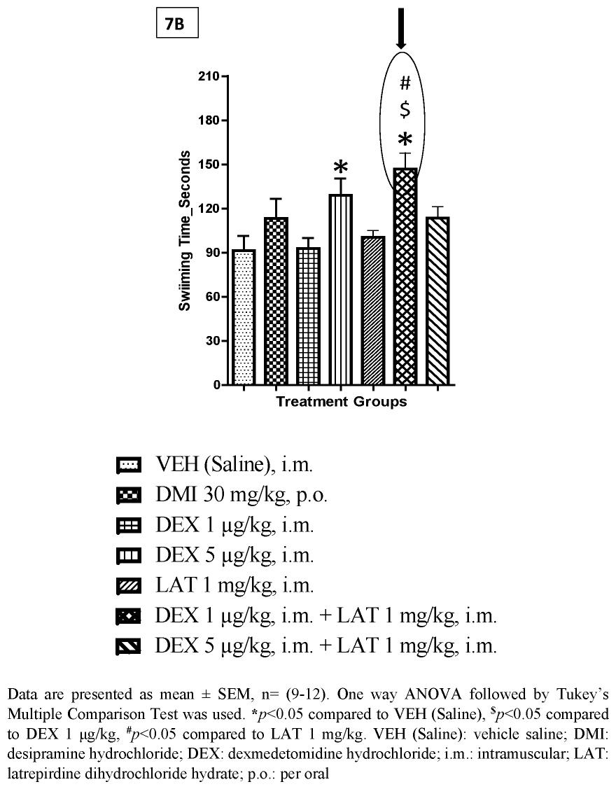

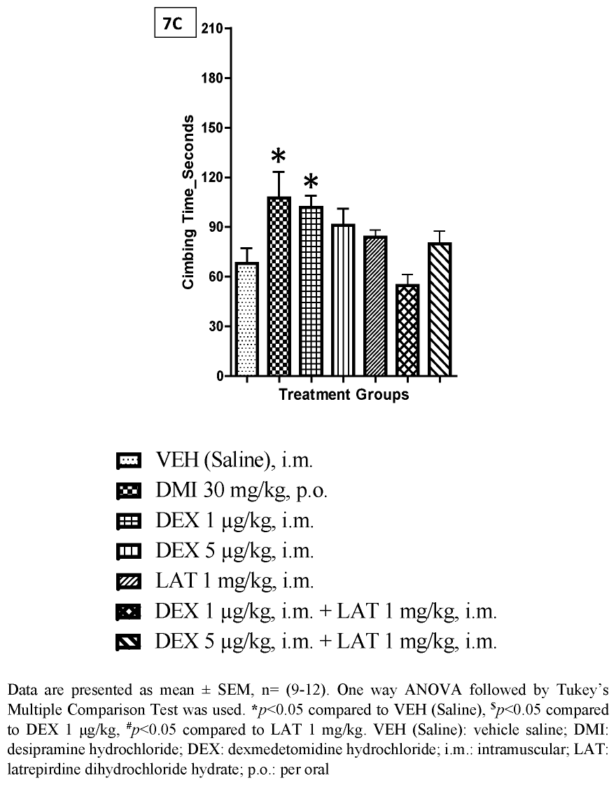

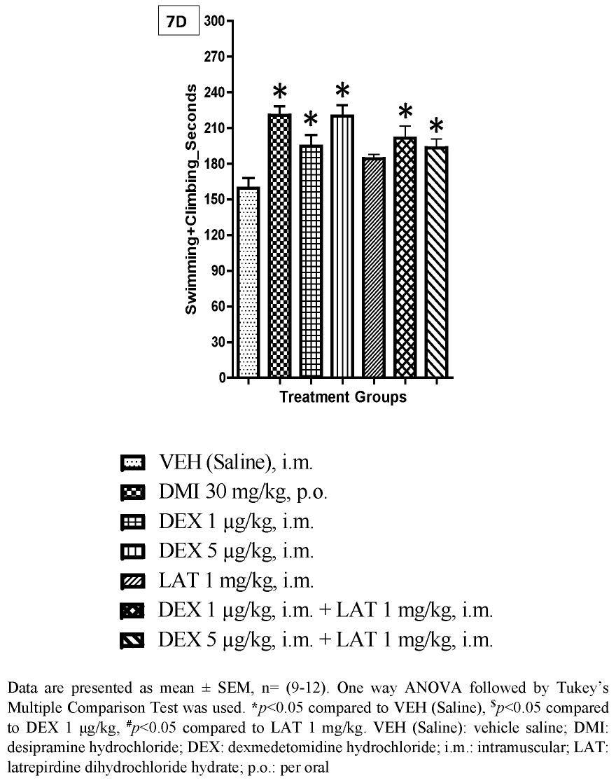

The effects of treatment drugs (desipramine hydrochloride, dexmedetomidine hydrochloride, and latrepirdine dihydrochloride hydrate) on immobility, swimming and climbing are shown in FIG. 7A-D.

The standard antidepressant drug i.e. desipramine hydrochloride (30 mg/kg, p.o.), showed a significant decrease in immobility time and an increase in climbing time as compared to vehicle saline group during 5 minutes observation on FST test day. However, the treatment of desipramine hydrochloride (30 mg/kg, p.o.) failed to show any significant effect on swimming time, suggesting the involvement of noradrenergic mechanism in desipramine mediated anti-depressant effect in FST rat model (Detke et al., 1995) (see FIGS. 7A-D).

Treatment of dexmedetomidine hydrochloride (1 and 5 μg/kg, i.m.) significantly decreased the immobility time as compared to vehicle saline group. Further, dexmedetomidine hydrochloride (1 μg/kg, i.m.) significantly increased climbing time as compared to vehicle saline group. Moreover, dexmedetomidine hydrochloride (5 μg/kg, i.m.) significantly increased swimming time that indicates it produces an anti-depressant effect (see FIG. 7C).

Treatment with the combination of dexmedetomidine hydrochloride (1 μg/kg, i.m.) and Latrepirdine dihydrochloride hydrate (1 mg/kg, i.m.) significantly increased swimming time (˜64.3%) as compared with vehicle saline group, while increase in swimming time for the individual drugs were ˜1.6% by dexmedetomidine hydrochloride (1 μg/kg, i.m.) and 9.8% by latrepirdine dihydrochloride hydrate (1 mg/kg, i.m.) as compared to vehicle saline, suggesting the synergistic interaction between these two drugs at that particular dose combination. The effect of combination drug treatment on swimming behaviour could be due to an increase in the brain serotonin level, a neurotransmitter known to have a mood elevation effect (see FIG. 7B).

CONCLUSION: It may be concluded that the synergy observed between dexmedetomidine hydrochloride and latrepirdine dihydrochloride hydrate at a particular dose (1 μg/kg and 1 mg/kg, respectively) is arising out of specific brain levels of Norepinephrine (NE) and Serotonin effected by those doses of the drugs.

The above study evaluated the antidepressant-like efficacy of dexmedetomidine hydrochloride (1 and 5 μg/kg, i.m.) alone or in combination with latrepirdine dihydrochloride hydrate (1 mg/kg, i.m.) using the Forced Swim test rat model in dark phase. The combination resulted in a significant increase in swimming behavior which is even greater than that of 5 μg/kg, i.m. dexmedetomidine hydrochloride, showing synergistic effect of two sub-optimal doses of dexmedetomidine hydrochloride and latrepirdine dihydrochloride hydrate individually.

### Example 5. To Evaluate the Effect of Dexmedetomidine Hydrochloride, Latrepirdine Dihydrochloride Hydrate and their Combination on Aggressive Behavior of Male Swiss Albino Mice in Resident Intruder Task

Materials and Methods

Test system: Mice

Strain/Gender: Swiss albino/Male

Source: Vivo Bio Tech Ltd. Hyderabad, Telangana, India Group allocation

TABLE 10
Treatment schedule

| Group | | | |
|---|---|---|---|
| Number | n | Treatment Group | Dose and Route |
| 1 | 14 | Vehicle | 10 mL/kg, i.p |
| 2 | 11 | Dexmedetomidine Hydrochloride | 4 μg/kg, i.p. (10 mL/kg, i.p) |
| 3 | 7 | Dexmedetomidine Hydrochloride | 10 μg/kg, i.p. (10 mL/kg, i.p) |
| 4 | 9 | Latrepirdine dihydrochloride hydrate | 0.3 mg/kg, i.p. (10 mL/kg, i.p.) |
| 5 | 9 | Latrepirdine dihydrochloride hydrate | 1 mg/kg, i.p. (10 mL/kg, i.p.) |
| 6 | 7 | Latrepirdine dihydrochloride hydrate | 3 mg/kg, i.p. (10 mL/kg, i.p.) |
| 7 | 7 | Latrepirdine dihydrochloride hydrate | 10 mg/kg, i.p. (10 mL/kg, i.p.) |
| 8 | 9 | Dexmedetomidine Hydrochloride + | 4 μg/kg, i.p. + 0.3 mg/kg, i.p. |
| | | Latrepirdine dihydrochloride hydrate | (10 mL/kg, i.p.) |
| 9 | 9 | Dexmedetomidine Hydrochloride + | 4 μg/kg, i.p. + 1 mg/kg, i.p. |
| | | Latrepirdine dihydrochloride hydrate | (10 mL/kg, i.p.) |
| 10 | 7 | Dexmedetomidine Hydrochloride + | 4 μg/kg, i.p. + 3 mg/kg, i.p. |
| | | Latrepirdine dihydrochloride hydrate | (10 mL/kg, i.p.) |
| 11 | 7 | Dexmedetomidine Hydrochloride + | 4 μg/kg, i.p. + 10 mg/kg, i.p. |
| | | Latrepirdine dihydrochloride hydrate | (10 mL/kg, i.p.) |
| 12 | 6 | Dexmedetomidine Hydrochloride + | 10 μg/kg, i.p. + 1 mg/kg, i.p. |
| | | Latrepirdine dihydrochloride hydrate | (10 mL/kg, i.p.) |
| 13 | 10 | Sodium valproate | 300 mg/kg, i.p. (10 mL/kg, i.p.) |

* weight was calculated based on free base.

Total number of animals: 280 males and 140 females.

Age at study: 3-6 weeks

Body weight range: ˜25-40 g (140 resident males)/˜10-20 g (140 intruder males)/15-25 g (140 females)

Veterinary examination: Prior to the final assignment to the study, mice were subjected to veterinary examination to ensure that the selected mice were in a good state of health.

Identification: Tail marking with permanent marker.

Acclimatization: After health examination, animals were acclimatized for a period of one week under test conditions. Only animals without any visible signs of illness were used for the study.

Husbandry

Conditions: Standard laboratory conditions, temperature were maintained between 21±3° C. and relative humidity between 30-70% under a 12 h light/dark cycle (lights on between 07:00-19:00 h)

Accommodation: All the resident mice were housed individually (1 animal/cage) and intruder mice were housed socially (5 animals/cage) in sterilized solid bottom polycarbonate cages (Dimensions 17 inches (L)×10 inches (W)×8 inches (H) with stainless steel grill tops, facilities for food and water bottle, and bedding of clean Corn cob. The cages were suspended on stainless steel racks.

Diet: Pelleted rodent SAFE™ Laboratory diet manufactured by SAFE, France, were provided.

Water: Potable water passed through water filtration system were provided ad libitum in polycarbonate bottles with stainless steel sipper tubes.

Animal handling: Experiment were carried out by qualified and trained scientific personnel's (trained in animal handling, drug administration and experimentation).

Safety Precautions: Routine hygienic procedures (aprons, gloves, goggles, face mask and head caps) were followed.

Dose Formulation: The test substance was dissolved in 0.9% normal saline which served as the vehicle.

Vehicle of compound: Dexmedetomidine hydrochloride and latrepirdine dihydrochloride hydrate*: 0.9% normal saline.

Formulation preparation of Sodium valproate: Sodium valproate was dissolved in 0.9% normal saline.

Formulation preparation of β-Estradiol: β-Estradiol was dissolved in 0.9% normal saline.

Formulation appearance: Dexmedetomidine hydrochloride and latrepirdine dihydrochloride hydrate (clear solution), sodium valproate (clear solution), β-Estradiol (clear solution).

* Note for Latrepirdine: Vortexing and sonication for 5 minutes may be applied to obtain clear solution.

Stability of Formulation:

All formulations were freshly prepared on day of treatment. dexmedetomidine hydrochloride and latrepirdine dihydrochloride hydrate formulations were stored at 4° C., covered from the light until administration.

Treatment Method: i.p

Duration of acclimatization: At least one week

Frequency of administration: Once

Dose volume: 10 mL/kg

### Experimental Procedure

Bilateral ovariectomy surgery was carried out in 3-4 weeks old female Swiss albino mice as per the standard methods (Alagwu and Nneli, 2005). Animals were anesthetized using Avertin (2,2,2-Tribromoethanol) at 250 mg/kg, i.p. and were laid down on the surgery table and fixed with adhesive plaster. A midline incision was made on the dorsal region below the rib and 1 cm lateral to the either side of midline, a small incision was made on fascia to isolate the adipose fat supporting the ovaries. By slowly pulling out the fat tissue, the ovary was identified and excised the following uterine horn ligation with silk sutures. Fascia was covered with sutures and similar procedure was repeated on the other side as well. Superficial skin layers were sutured and gentamicin (80 mg/kg, s.c.) was given as antibiotic and meloxicam (4 mg/kg, s.c.) as analgesic, then povidone iodine was applied on superficial skin layers. Polyethylene glycol (PEG) was applied to prevent drying of eye balls and kept for recovery period of 3 weeks (surgery was carried by a single experimenter). Male Swiss albino mice (25-40 grams, Resident animals) was housed individually i.e., 1 animal/cage with an ovariectomized female Swiss albino mice for a period of three weeks (Isolated habituation). During this habituation period, female mice were treated with β-Estradiol at a dose of 0.2 mg/kg, s.c. Male Swiss albino mice (10-20 grams, Intruder animals) were housed 5 animals/cage for a period of five days (Social habituation). On the day of exposure, animals were brought into the experimental room 60 minutes before starting the experiment, to habituate them to the experimental conditions. On day 1 and 2, intruder was placed in the home cage of resident animal for a period of 10 minutes and returned to their home cages. During this 10 minutes exposure, the aggressive behavior (tail rattling, chasing, biting, lateral attack, clinch attack) of resident animal was noted as duration of attack along with latency of attack. During this exposure, female mice were removed from the resident cage (selection phase were carried out by 3 experimenters between 9 AM and 1 PM).

Animals that met the following criteria were selected for treatment.

Average duration of attack (day 1 & 2) of resident animal should be more than 30 seconds. Selected animals were randomized into different groups based on their duration of attack.

On day 4 from initial exposure, Test item/Vehicle/Sodium valproate were administered to the resident animal and same intruder was exposed to the same resident animal 60 min prior to trial. Sodium valproate was administered to the resident animal 30 min prior to trial

Statistical Analysis:

Data obtained was compared with basal scoring by using Student's paired two tailed t-test. Duration of attack, and latency of attack between the groups was analyzed using One-Way ANOVA followed by Bonferroni's posthoc test. All statistical analysis were performed using Graph pad prism software package (Version 7 or Higher) and p value below 0.05 was considered as significant.

Observations: The observations regarding mortality and clinical signs were monitored and reported.

### Results:

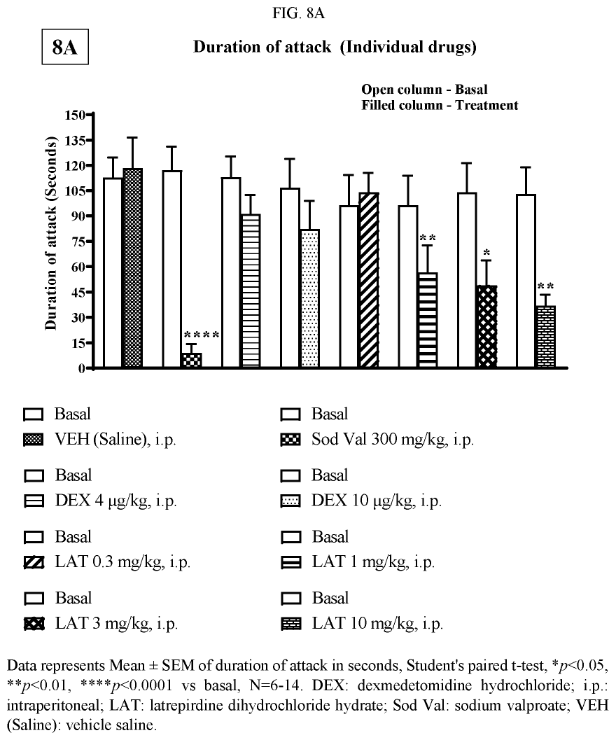

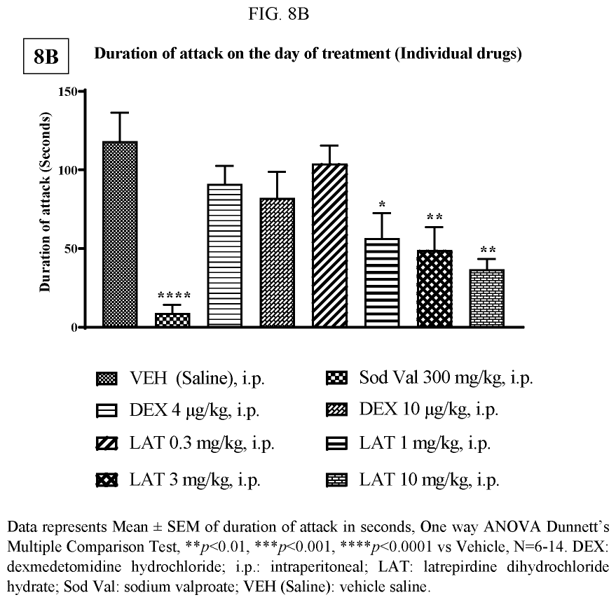

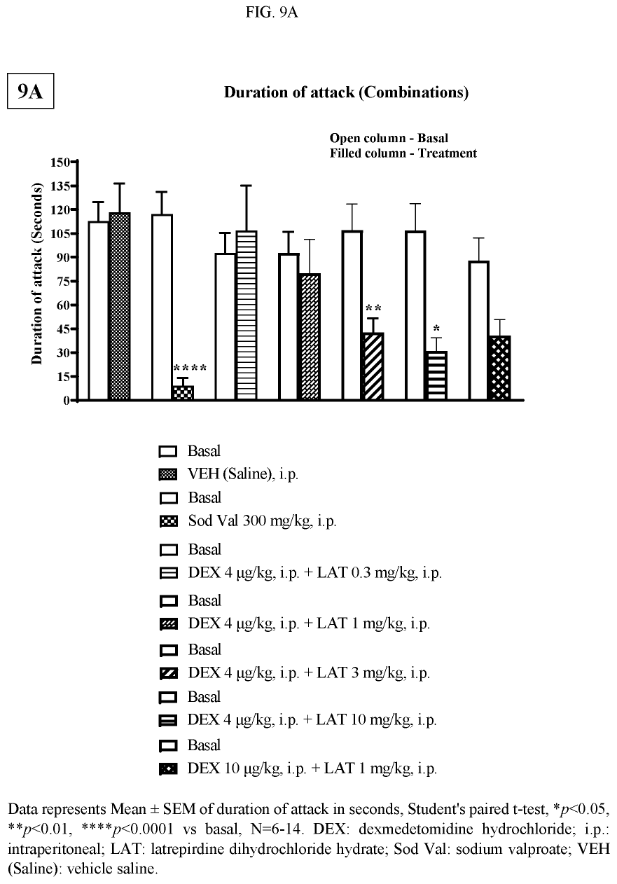

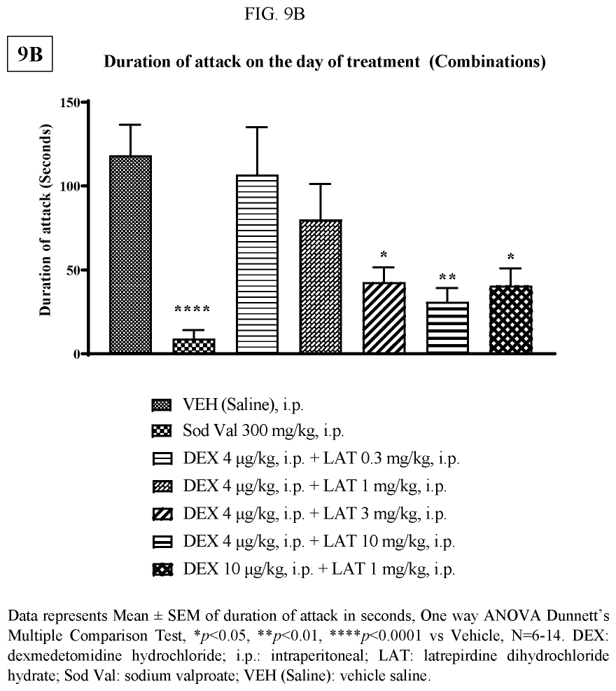

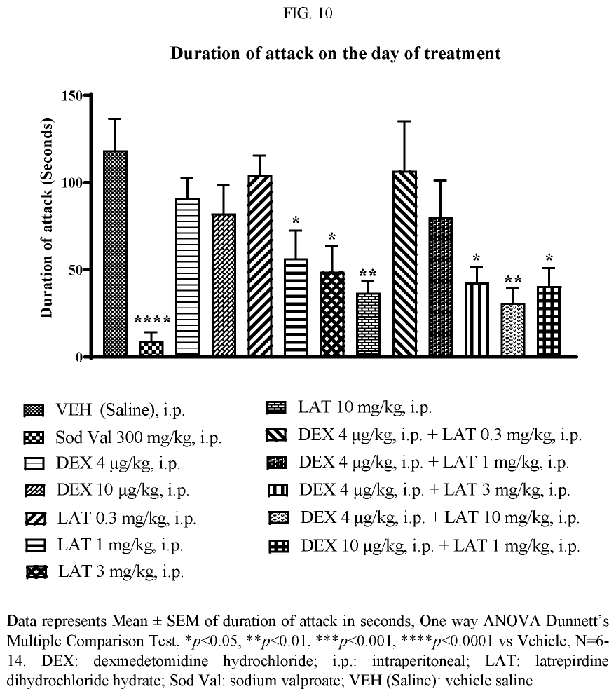

It was found that latrepirdine dihydrochloride hydrate decreased agitation/aggression in resident mice. In the FIGS. 8A & 8B, latrepirdine dihydrochloride hydrate (1 mg/kg, 3 mg/kg and 10 mg/kg; i.p.) significantly decreased the duration of attack. Similarly, few combinations of dexmedetomidine hydrochloride and latrepirdine dihydrochloride hydrate (dexmedetomidine 4 mcg/kg, i.p.+latrepirdine 3 mg/kg, i.p.; dexmedetomidine 4 mcg/kg, i.p.+latrepirdine 10 mg/kg, i.p.; and dexmedetomidine 10 mcg/kg, i.p.+latrepirdine 1 mg/kg, i.p.) were found to have significant effect on reduction in duration of attack compared to vehicle (FIGS. 9A & 9B). Two combinations of dexmedetomidine and latrepirdine, namely, dexmedetomidine 4 ng/kg, i.p.+latrepirdine 3 mg/kg, i.p. and dexmedetomidine 10 ng/kg, i.p.+latrepirdine 1 mg/kg, i.p. showed nearly same efficacy in reducing the duration of attack as that of latrepirdine 10 mg/kg, i.p. with mean values of duration of attack being 42.58 sec, 40.73 sec and 36.87 sec, respectively (FIG. 10). These two combinations could be useful from drug safety point of view, where reduced doses of each of the individual components can be used as a combination instead of a higher dose of a single compound alone with similar efficacy.

### Example 6: Antipsychotic Effect of Dexmedetomidine Hydrochloride and Latrepirdine in Mice Using Smart-Cube System

Dexmedetomidine and latrepirdine have anti-psychotic effects, when tested in the SmartCube® system (Psychogenics, Inc., Paramus, NJ; See also U.S. Pat. No. 7,580,798 incorporated herein by reference in its entirety). This system uses features derived from mouse behavioral data to first, classify mice on compound versus vehicle. This measure indicates how well the system can differentiate between a mouse on compound versus a mouse on vehicle. Secondly, by comparing the features to a proprietary reference database of behavioral feature sets that are linked to classes of marketed drugs known to treat neuro-psychiatric symptoms SmartCube® assigns a signature to the behavior. Thus, the system can be used as a model to identify both the activity and the psychiatric effect of a compound by comparing the effects of the compounds against drugs with known validated effects.

By comparing the responses of animals to known drugs, the test drug can be categorized according to its function; for example, hallucinogen, anxiogenic, analgesic, cognitive enhancer, psychostimulant, mood stabilizer, high dose anti-psychotic, anti-psychotic, sedative/hypnotic, anxiolytic, high dose antidepressant, antidepressant.

Once all features are extracted from the raw data through an automated pipeline, proprietary bioinformatics algorithms are used to decorrelate groups of features and find the combination of values that best separate different groups of interest. For each compound, at each dose, they system provides a probability that the drug is active and breaks down such putative activity into the different classes of interest.

SmartCube reference data used herein include anti-psychotics and anti-depressants tested at several dose ranges. “Anti-psychotic” versus “high dose anti-psychotic” as indicated in the legend, reflects the notion that anti-psychotics reference data is dose dependent. Anti-psychotics, when administered at higher doses, can engage additional receptor systems, and thus affect mouse behavior differently. The same applies for different doses of anti-depressants.

Materials and Methods:

Animals: Male C57/B16 mice (N=12 per group) from Taconic Laboratories were used. Upon receipt, mice were group-housed in OPTI mouse ventilated cages with 4 mice per cage. Mice were acclimated to the colony room for at least one week prior to test and subsequently tested at approximately 8-9 weeks of age. All animals were examined, handled, and weighed prior to initiation of the study to assure adequate health and suitability and to minimize non-specific stress associated with manipulation.

During the study, 12/12 light/dark cycles were maintained. The room temperature was maintained between 20 and 23° C. with a relative humidity maintained around between 30-70%. Chow and water were provided ad libitum for the duration of the study.

Animals were acclimated to the vivarium for up to one week prior to commencing study. Room temperature and humidity was recorded continuously in the holding room. The experimenter(s) were blind to the treatment distribution. The behavioral tests were conducted according to established protocols approved by the IACUC committee and PGI's Standard Operation Procedures (SOP). The standard safety precautions were applied to all studies. Personnel working in the animal room and laboratory wore protective clothing.

Treatment: 8 groups (N=12 per group)

All compounds tested were formulated in NP3 (vehicle solution): 5% Pharmasolve; 30% P3 (1:1:1 PEG200:PEG400: propylene glycol); 65% saline; pH is 5.1-6.

All SmartCube runs were conducted with NP3 as vehicle and under identical settings.

Test groups were:
Vehicle: NP3
Dexmedetomidine at 0.005 and 0.010 mg/kg
Latrepirdine at 1 and 10 mg/kg
Dexmedetomidine and latrepirdine combinations (in mg/kg): 0.005/1.0; 0.005/10; 0.010/1.0; 0.010/10

Test compounds were injected intraperitoneally (IP) for 15 min before animals were placed in SmartCube for assessment.

Explanation of SmartCube legend:

Vehicle: The activity profile of mice injected Intraperitonealy (IP) with vehicle (NPS) Antipsychotic: SmartCube classifies the mouse behavior with test compound as similar to treatment with marketed anti-psychotics at therapeutically relevant doses.

High Dose Antipsychotic: SmartCube classifies the mouse behavior on test compound as

similar to treatment with marketed anti-psychotics at doses that are considered as high therapeutically. High doses of anti-psychotics often cause sedation.

Antidepressant: SmartCube classifies the mouse behavior with test compound as similar to treatment with marketed anti-depressants at therapeutically relevant doses.

High dose Antidepressant: SmartCube classifies the mouse behavior on test compound as similar to treatment with marketed anti-depressants at doses that are considered high therapeutically.

Mood Stabilizer: SmartCube classifies the mouse behavior on test compound as similar to treatment with marketed mood stabilizers at doses that are considered high therapeutically

Results:

The results for the class analyses are presented as standardized bar charts with percentages that sum to 100 for each dose. The percentage indicates the probability the classifier can differentiate between the vehicle group and test group. The pattern indicates what class signature was assigned.

Dexmedetomidine has an antipsychotic signature in SmartCube.

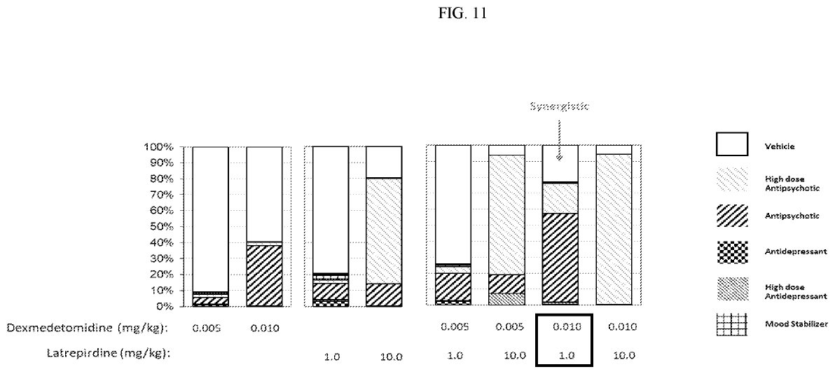

Smart-Cube signatures from mice dosed (IP mg/kg; N=12 per group) at 2 doses (0.005 and 0.010 mg/kg) of dexmedetomidine, an alpha2-adrenergic receptor agonist (FIG. 11). Smart-Cube deep learning classifiers assign activity signatures by comparing phenotypic behavior of mice injected with dexmedetomidine to a library of reference data obtained with known compounds. At 0.005 an 0.010 mg/kg, Smart-Cube classifies the dexmedetomidine group with increased accuracy (compared to vehicle) and assigns an antipsychotic signature.

Latrepirdine has an antipsychotic signature in SmartCube.

Smart-Cube signatures from mice dosed (IP mg/kg; N=12 per group) at 2 doses (1.0 and 10 mg/kg) of latrepirdine. (FIG. 11). Smart-Cube deep learning classifiers assign activity signatures by comparing phenotypic behavior of mice injected with latrepirdine to a library of reference data obtained with known compounds. At 1 and 10 mg/kg, Smart-Cube classifies the latrepirdine group with increased accuracy (compared to vehicle) and assigns an antipsychotic signature.

Synergism at 0.010 mg/kg dexmedetomidine and 1 mg/kg of latrepirdine.

Smart-Cube signatures from mice dosed (IP mg/kg; N=12 per group) with combinations of dexmedetomidine and latrepirdine: 0.005/1.0; 0.005/10; 0.010/1.0; 0.010/10 (dexmedetomidine/latrepirdine; mg/kg; FIG. 11). Smart-Cube deep learning classifiers assign activity signatures by comparing phenotypic behavior of mice injected with dexmedetomidine and latrepirdine combinations to a library of reference data obtained with known compounds. At dose of 0.005/1.0 (dexmedetomidine/latrepirdine in mg/kg): Smart-Cube assigns an antipsychotic signature and a total activity that is roughly the sum (25%) of each dose separately (9% and 20% individual doses of dexmedetomidine and latrepirdine respectively). At doses of 0.005/10 (dexmedetomidine/latrepirdine in mg/kg): Smart-Cube assigns a high dose antipsychotic signature and a total activity that is roughly the sum (90%) of each dose separately (9% and 80%, individual doses of dexmedetomidine and latrepirdine respectively). At doses of 0.010/1.0 (dexmedetomidine/latrepirdine in mg/kg): Smart-Cube assigns an antipsychotic signature, plus some high dose antipsychotic signature with a total activity that is greater than the sum of each dose separately (40% and 20% for the individual doses of dexmedetomidine and latrepirdine respectively). The sum is 60%, but the dose combination effect is 76%). At doses of 0.01/10 (dexmedetomidine/latrepirdine in mg/kg): Smart-Cube assigns a high dose antipsychotic signature and a total activity that maxes out at 95%

These data indicate that at suboptimal doses dexmedetomidine (at 0.010 mg/kg and latrepirdine (at 1 mg/kg) act synergistically while mostly preserving the anti-psychotic signature.

### Example 7. To Evaluate the Effect of Dexmedetomidine Hydrochloride, Latrepirdine Dihydrochloride Hydrate and their Combination on Motor Activity (Locomotion) and Sedation Behavior of Male Swiss Albino Mice in Open Field Test

### Materials and Methods

### Test System

Test system: Mice
Strain/Gender: Swiss albino/Male
Study: Open field test

Open field test: The spontaneous locomotor activity in terms of total distance traveled is measured in open field test. Comparatively lower distance traveled would indicate either less motor activity or sedation.

Group allocation:

TABLE 11
Treatment schedule

| Group | N per | | |
|---|---|---|---|
| No. | group | Treatment group | Dose/Route/Dosing volume |
| 1 | 12 | Vehicle | 10 mL/kg, i.p |
| 2 | 12 | Dexmedetomidine hydrochloride | (4 μg/kg, i.p.) (10 mL/kg, i.p.) |
| 3 | 12 | Dexmedetomidine hydrochloride | (10 μg/kg, i.p.) (10 mL/kg, i.p.) |
| 4 | 12 | Dexmedetomidine hydrochloride | (20 μg/kg, i.p.) (10 mL/kg, i.p.) |
| 5 | 12 | Dexmedetomidine hydrochloride | (30 μg/kg, i.p.) (10 mL/kg, i.p.) |
| 6 | 12 | Latrepirdine dihydrochloride hydrate | (1 mg/kg, i.p.) (10 mL/kg, i.p.) |
| 7 | 12 | Latrepirdine dihydrochloride hydrate | (3 mg/kg, i.p.) (10 mL/kg, i.p.) |
| 8 | 12 | Latrepirdine dihydrochloride hydrate | (10 mg/kg, i.p.) (10 mL/kg, i.p.) |
| 9 | 12 | Dexmedetomidine hydrochloride + | (4 μg/kg, i.p.) + (10 mg/kg, i.p.) |
| | | Latrepirdine dihydrochloride hydrate | |
| 10 | 12 | Dexmedetomidine hydrochloride + | (10 μg/kg, i.p.) + (1 mg/kg, i.p.) |
| | | Latrepirdine dihydrochloride hydrate | |

TOTAL number of animals: 120 males
Age at study: 6-7 weeks
Body weight range: ˜25-40 g

Acclimatization: After health examination, animals were acclimatized for a period of one week under test conditions. Only animals without any visible signs of illness were used for the study.

Husbandry

Conditions: Standard laboratory conditions, temperature was maintained between 21±3° C. and relative humidity between 30-70% under a 12 h light/dark cycle (lights on between 07:00-19:00 h).

Accommodation: All the mice were housed in groups of 4) (Dimensions 17 inches (L)×10 inches (W)×8 inches (H) with stainless steel grill tops, facilities for food and water bottle, and bedding of clean Corn cob. The cages were suspended on stainless steel racks.

Diet: Pelleted rodent SAFE™ Laboratory diet manufactured by SAFE, France, was provided.

Water: Potable water passed through water filtration system was provided ad libitum in polycarbonate bottles with stainless steel sipper tubes.

Animal handling: Experiment was carried out by qualified and trained scientific personnel's (trained in animal handling, drug administration and experimentation)

Safety Precautions: Routine hygienic procedures (aprons, gloves, goggles, face mask and head caps) were followed.

Dose Formulation

The test substance was dissolved in 0.9% normal saline which served as the vehicle.

Vehicle of dexmedetomidine hydrochloride and latrepirdine dihydrochloride hydrate*: 0.9% normal saline.

Formulation appearance: Dexmedetomidine hydrochloride and latrepirdine dihydrochloride hydrate (Clear solution),

* Note for latrepirdine dihydrochloride hydrate: Vortexing and sonication for 5 minutes may be applied to obtain clear solution.

Stability of Formulation

All formulations were freshly prepared on day of treatment. Dexmedetomidine hydrochloride and latrepirdine dihydrochloride hydrate formulations were stored at 4° C., cover from the light until administration.

Experimental Procedure

Mice were brought to the laboratory and allowed to acclimatize to the environment for at least 7 days. The open field test was done in lighting conditions of 40 lux light. The mice were dosed as per the allocation. After a post dose interval of 60 minutes, 4 mice from a group were placed in the open field and the movement was tracked for a period of 30 minutes. Once the trial was over the mice were removed from the open field and the arena was cleaned. Following this, next set of 4 mice were placed in the arena and tracked.

Statistical Analysis

The distance travelled for a period of 30 minutes was analyzed in bins of 5 minutes and was compared with that of the vehicle group using one-way ANOVA followed by Dunnett's test. All statistical analysis were performed using Graph pad prism software package (Version 7 or Higher) and p value below 0.05 was considered as significant.

Results:

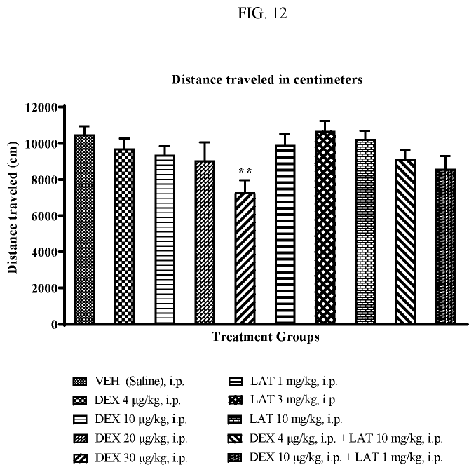

Dexmedetomidine hydrochloride (at 4 μg/kg, 10 μg/kg and 20 μg/kg) did not show any significant reduction in distance traveled (FIG. 12). In addition, latrepirdine dihydrochloride hydrate tested at 1 mg/kg, 3 mg/kg and 10 mg/kg did not show any significant reduction in distance traveled. Further, combinations of dexmedetomidine hydrochloride and latrepirdine dihydrochloride hydrate (4 μg/kg+10 mg/kg and 10 μg/kg+1 mg/kg, respectively) also did not show any significant reduction in distance traveled. Whereas dexmedetomidine hydrochloride only at 30 μg/kg showed significant reduction in distance traveled.

1. A method of treating agitation in an agitated subject, comprising oromucosally administering to the subject a therapeutically effective amount of dexmedetomidine or a pharmaceutically acceptable salt thereof in combination with a therapeutically effective amount of latrepirdine or a pharmaceutically acceptable salt thereof.

2. A method of treating agitation in an agitated subject, comprising oromucosally administering to the subject a dosage form comprising a therapeutically effective amount of latrepirdine or a pharmaceutically acceptable salt thereof.

3. A method of treating depression in a subject in need thereof, comprising administering to the subject a therapeutically effective amount of dexmedetomidine or a pharmaceutically acceptable salt thereof in combination with a therapeutically effective amount of latrepirdine or a pharmaceutically acceptable salt thereof.

4. A method of treating depression in a subject in need thereof, comprising oromucosally administering to the subject a dosage form comprising a therapeutically effective amount of latrepirdine or a pharmaceutically acceptable salt thereof.

5. A method of treating psychosis in a subject in need thereof, comprising administering to the subject a therapeutically effective amount of dexmedetomidine or a pharmaceutically acceptable salt thereof in combination with a therapeutically effective amount of latrepirdine or a pharmaceutically acceptable salt thereof.

6. A method of treating psychosis in a subject, comprising oromucosally administering to the subject a dosage form comprising a therapeutically effective amount of latrepirdine or a pharmaceutically acceptable salt thereof.

7. The method according to claim 1, wherein dexmedetomidine and latrepirdine or pharmaceutically acceptable salts thereof are administered sequentially in separate dosage forms.

8. The method according to claim 1, wherein dexmedetomidine and latrepirdine or pharmaceutically acceptable salts thereof are formulated in single dosage form.

9. The method according to any of claims 2, 4, and 6-8, wherein the dosage form is administered once a day.

10. The method according to any of claims 2, 4 and 6-8, wherein the dosage form is administered multiple times a day.

11. The method according to claims 2, 4, and 6-10, wherein the dosage form is a lyophilized oromucosal tablet, and is preferably administered sublingually or buccally.

12. The method according to claims 1 to 2, wherein the agitation is associated with a neurodegenerative disorder selected from the group consisting of Alzheimer's disease, frontotemporal dementia (FTD), dementia, dementia with Lewy bodies (DLB), post-traumatic stress disorder, Parkinson's disease, vascular dementia, vascular cognitive impairment, Huntington's disease, multiple sclerosis, Creutzfeldt-Jakob disease, multiple system atrophy, and progressive supranuclear palsy.

13. The method according to claims 1 to 2, wherein the agitation is associated with a neuropsychiatric disorder selected from the group consisting of schizophrenia, bipolar disorder, bipolar mania, delirium, and depression.

14. The method according to claims 1 to 2, wherein the agitation is associated with an alcohol, opioid use disorder, opioid withdrawal and substance use withdrawal.

15. The method according to claims 1 to 2, wherein the agitation is caused by noradrenergic mediated hyperarousal.

16. The method according to claims 1 to 2, wherein the treatment is effective to suppress agitation in an agitated subject without also causing significant sedation.

17. The method according to claims 1 to 2, wherein the agitation is acute or chronic.

18. An oromucosal dosage form comprising:
(i) a therapeutically effective amount of latrepirdine or a pharmaceutically acceptable salt thereof;
(ii) one or more mucoadhesive agents and
(iii) one or more pharmaceutically acceptable excipients or carriers, wherein the dosage form disintegrates in not less than 5 minute upon contact with an oral mucosa.

19. An oromucosal dosage form comprising:
(i) a therapeutically effective amount of latrepirdine or a pharmaceutically acceptable salt thereof;
(ii) a therapeutically effective amount of dexmedetomidine or a pharmaceutically acceptable salt thereof;
(iii) one or more mucoadhesive agents and
(iv) one or more pharmaceutically acceptable excipients or carriers, wherein the dosage form disintegrates in not less than 1 minute upon contact with an oral mucosa.

20. The oromucosal dosage form according to claims 18 to 19, wherein one or more mucoadhesive agents are present in an amount of about 0.5% to about 30% w/w.

21. The oromucosal dosage form according to claims 18 to 20, wherein the dosage form is prepared by spray drying, sublimation, nanonization, granulation, direct compression or lyophilization, preferably lyophilization.

22. The oromucosal dosage form according to claims 18 to 21, wherein the dosage form has a mucoadhesive peak force greater than about 50 g, about 100 g, about 200 g, about 300 g, about 400 g, about 500 g, about 600 g, about 700 g, about 800 g, about 900 g, about 1000 g, about 1100 g, about 1200 g, about 1300 g, about 1400 g or about 1500 g.

23. The oromucosal dosage form according to any of claims 18 to 22, wherein the dosage form is a sublingual tablet.

24. The oromucosal dosage form according to any of claims 18 to 22, wherein the dosage form is a buccal or gingival tablet.

25. The oromucosal dosage form according to claim 18, wherein the dosage form is a lyophilized tablet comprising:
(i) sodium alginate; (ii) croscarmellose sodium or sodium starch glycolate; (iii) sucralose; (iv) magnesium stearate and/or silicon dioxide; (v) lactose or mannitol; and (vi) optionally other pharmaceutical acceptable excipients.

26. The oromucosal dosage form according to claim 18, wherein the dosage form is a lyophilized tablet comprising:
(i) carbomer; (ii) croscarmellose sodium or sodium starch glycolate; (iii) sucralose; (iv) magnesium stearate and/or silicon dioxide; (v) lactose or mannitol; and (vi) optionally other pharmaceutical acceptable excipients.

27. The oromucosal dosage form according to claim 18, wherein the dosage form is a lyophilized tablet comprising:
(i) xanthan gum; (ii) croscarmellose sodium or sodium starch glycolate; (iii) sucralose; (iv) magnesium stearate and/or silicon dioxide; (v) lactose or mannitol; and (vi) optionally other pharmaceutical acceptable excipients.

28. The oromucosal dosage form according to claim 18, wherein the dosage form is a lyophilized tablet comprising:
(i) carbomer, hydroxypropyl cellulose, hydroxypropyl methylcellulose, or polyethylene oxide; (ii) croscarmellose sodium, or sodium starch glycolate; (iii) sucralose, (iv) magnesium stearate and/or silicon dioxide; (v) lactose or mannitol; and (vi) optionally other pharmaceutical acceptable excipients.

29. The oromucosal dosage form according to claim 18, wherein the dosage form is a lyophilized tablet comprising:
(i) sodium alginate, xanthan gum, carbomer, hydroxypropyl cellulose, hydroxypropyl methylcellulose, or polyethylene oxide; (ii) croscarmellose sodium or sodium starch glycolate; (iii) sucralose; (iv) magnesium stearate and/or silicon dioxide; (v) lactose or mannitol; and (vi) optionally other pharmaceutical acceptable excipients.

30. A kit comprising an oromucosal lyophilized tablet dosage form comprising:
(i) a therapeutically effective amount of dexmedetomidine or a pharmaceutically acceptable salt thereof (e.g. hydrochloride salt);
(ii) a therapeutically effective amount of latrepirdine or a pharmaceutically acceptable salt thereof (e.g., hydrochloride salt); and optionally
(iii) instructions for the administration of said dosage form to a subject in need thereof.

31. The oromucosal dosage form/method/kit according to any of the preceding claims, wherein latrepirdine or a pharmaceutically acceptable salt thereof is latrepirdine hydrochloride or latrepirdine dihydrochloride.

32. The oromucosal dosage form/method/kit according to any of the preceding claims, wherein dexmedetomidine or a pharmaceutically acceptable salt thereof is dexmedetomidine hydrochloride.

33. The oromucosal dosage form/method/kit according to any of the preceding claims, wherein the therapeutically effective amount of dexmedetomidine or a pharmaceutically acceptable salt thereof is in the range from about 10 micrograms to about 300 micrograms.

34. The oromucosal dosage form/method/kit according to any of the preceding claims, wherein the therapeutically effective amount of latrepirdine or a pharmaceutically acceptable salt thereof is in the range from about 10 mg to about 100 mg.

35. The oromucosal dosage form according to any one of claims 20 to 27, wherein one or more mucoadhesive agents are selected from the group consisting of polyacrylic acid polymers, methacrylic acid polymers, cellulose derivatives such as hydroxyethyl cellulose, (HEC), hydroxypropyl cellulose (HPC), ethyl hydroxyethyl cellulose, hydroxypropyl methylcellulose (HPMC), methyl cellulose, sodium carboxymethyl cellulose, thiolated carboxymethyl cellulose; polysaccharides, xanthan gum, karaya gum, tragacanth gum, propylene glycol, propylene glycol alginate, sodium alginate, polyethylene oxide, microcrystalline cellulose (Avicel), croscarmellose and mixtures thereof.

36. The oromucosal dosage form according to claim 38, wherein the polyacrylic polymers are carbomers, polycarbophil or a combination thereof.

37. The oromucosal dosage form according to claim 38, wherein the polysaccharides are pectin, chitosan, or a combination thereof.

38. The oromucosal dosage form according to any one of claims 20 to 27, wherein one or more pharmaceutically acceptable excipients or carriers are selected from the group consisting of disintegrants, fillers/diluents, binders, glidants, lubricants, plasticizers, pH regulators, coloring agents, flavoring agents, sweetening agents and combinations thereof.

39. The oromucosal dosage form of any of the preceding claims, wherein the dosage form disintegrates within about 5 seconds to about 10 minutes upon contact with an oral mucosa.
